(19) United States
(12) Patent Application Publication
(10) Pub. No.: US 2025/0250620 A1
(43) Pub. Date: Aug. 7, 2025

(54) DETERMINISTIC BARCODING FOR SPATIAL PROFILING

(71) Applicant: Yale University, New Haven, CT (US)

(72) Inventors: Rong Fan, Cheshire, CT (US); Zhiliang Bai, New Haven, CT (US)

(73) Assignee: Yale University, New Haven, CT (US)

(21) Appl. No.: 19/045,525

(22) Filed: Feb. 4, 2025

Related U.S. Application Data
(63) Continuation of application No. 63/549,760, filed on Feb. 5, 2024.

(51) Int. Cl. C12Q 1/6841; C12Q 1/6851

(52) U.S. Cl. CPC C12Q 1/6841; C12Q 1/6851

(57) ABSTRACT

The disclosure relates to compositions and methods for spatial whole transcriptome sequencing in processed tissues.

### CROSS-REFERENCE TO RELATED APPLICATIONS

This application claims the benefit under 35 U.S.C. § 119 (e) of U.S. provisional application No. 63/549,760, filed Feb. 5, 2024, the entire contents of which are incorporated herein by reference.

### REFERENCE TO AN ELECTRONIC SEQUENCE LISTING

The contents of the electronic sequence listing (Y008770173US01-SEQ-GJM.xml; Size: 91,848 bytes; and Date of Creation: Jan. 31, 2025) are herein incorporated by reference in its entirety.

### BACKGROUND

Deterministic Barcoding in Tissue for Spatial Omics Sequencing (DBiT-seq) was developed to create a multi-omics approach for studying spatial gene expression heterogenicity within a tissue sample. DBiT-seq can be used for co-mapping mRNA and protein levels at a near single-cell resolution in fresh or frozen formaldehyde-fixed tissue samples, utilizing next generation sequencing and microfluidics to enable simultaneous spatial transcriptomic and proteomic analysis of a tissue sample. Yet, achieving high spatial resolution, genome wide, unbiased biomolecular profiling over a large area of processed tissue still has its challenges.

### SUMMARY

The present disclosure provides an improvement over the DBiT-seq technology to address distinctive challenges associated with analyzing genomic information in processed tissues, such as clinically archived formalin-fixed paraffin-embedded (FFPE) tissues. The technology described herein is referred to as Patho-DBiT. Clinically archived tissues (such as FFPE tissues) are stored at temperatures as low as −80° C. for as long as several decades. Long-term storage of such tissues often results in damage to nucleic acids within the tissues that poses a unique challenge for genomic analyses. Important genomic information in the form of ribonucleic acids (RNA) becomes fragmented within the tissues, which results in a significant loss of analyzable genomic data and limits the ability to conduct research on the tissues. Patho-DBIT, as described herein, overcomes this challenge by first polyadenylating RNA molecules, such as fragmented RNA molecules, which lack a poly(A) tail prior to conducting spatial transcriptomics analyses. The results described herein demonstrate the utility of Patho-DBIT for analyzing processed tissues, such as clinically archived FFPE tissues, which contain damaged genomic information, thus providing a path for researchers to analyze the abundant genomic information stored in these tissues.

Accordingly, in some aspects, a method of the disclosure, includes: (a) producing spatially barcoded complementary deoxyribonucleic acids (cDNAs) from polyadenylated fragmented ribonucleic acids (RNAs) in a tissue section obtained from processed tissue, such as FFPE tissue; and (b) mapping the spatially barcoded cDNAs to points of origin within the tissue section. It should be understood that the step of “mapping the spatially barcoded cDNAs” includes mapping all or only a subset of (at least one of) the spatially barcoded cDNAs to points of origin within the tissue section.

In some embodiments, a method includes: (i) delivering a polyadenylate polymerase to the tissue section, for example, delivering to the tissue section a polyadenylation reagent selected from polyadenylation specificity factors, cleavage stimulation factors, polyadenylate binding proteins, and cleavage factors; (ii) delivering reverse transcription reagents to the tissue section; and (iii) delivering to the tissue section a first set of barcoded polynucleotides, a second set of barcoded polynucleotides, and ligation reagents to produce spatially barcoded cDNAs.

In some aspects, a method includes: (a) polyadenylating fragmented RNAs in a tissue section obtained from processed tissue, such as FFPE tissue, to produce polyadenylated RNAs; (b) producing cDNAs from the polyadenylated RNAs; (c) spatially barcoding the cDNAs to produce spatially barcoded cDNAs; and (d) mapping the spatially barcoded cDNAs to points of origin within the tissue section. It should be understood that the step of “spatially barcoding the cDNAs” includes spatially barcoding all or only a subset of (at least one of) the cDNAs produced from the polyadenylated RNAs.

In some embodiments, fragmented RNAs are selected from the group consisting of mRNAs, ribosomal RNAs, transfer RNAs, microRNAs, long noncoding RNAs, small noncoding RNAs, small nuclear RNA, and piwi RNA.

In some embodiments, a method includes delivering a polyadenylate polymerase to the tissue section, and optionally delivering to the tissue section a polyadenylation reagent selected from polyadenylation specificity factors, cleavage stimulation factors, polyadenylate binding proteins, and cleavage factors.

In some embodiments, a method includes delivering reverse transcription reagents to the tissue section.

In some embodiments, a method includes delivering to the tissue section a first set of barcoded polynucleotides, a second set of barcoded polynucleotides, and ligation reagents to produce the spatially barcoded cDNAs.

In some aspects, a method includes: (a) delivering a polyadenylate polymerase to a tissue section obtained from processed tissue, such as FFPE tissue, to produce polyadenylated ribonucleic acids (RNAs); (b) delivering reverse transcription reagents to the tissue section to produce cDNAs; (c) delivering to the tissue section a first set of barcoded polynucleotides, a second set of barcoded polynucleotides, and ligation reagents to produce spatially barcoded cDNAs; (e) imaging the tissue section to produce a sample image; (f) sequencing the spatially barcoded cDNAs to produce sequencing reads; and (g) mapping the spatially barcoded cDNAs to points of origin within the tissue section. As noted above, it should be understood that the step of “mapping the spatially barcoded cDNAs” includes mapping all or only a subset of (at least one of) the spatially barcoded cDNAs to points of origin within the tissue section.

It should also be understood that the steps of the methods described herein need not be performed in the exact order indicated (e.g., denoted by a letter). For example, sequencing spatially barcoded cDNAs to produce sequencing reads can be performed before or simultaneously with imaging the tissue section to produce a sample image.

In some embodiments, a method includes delivering to the tissue section a polyadenylation reagent selected from polyadenylation specificity factors, cleavage stimulation factors, polyadenylate binding proteins, and cleavage factors.

In some embodiments, a first set of barcoded polynucleotides and a second set of barcoded polynucleotides are delivered using a microfluidic device, optionally made from polydimethylsiloxane (PDMS).

In some embodiments, s microfluidic device includes a first component for delivery of s first set of barcoded polynucleotides and a second component for delivery of s second set of barcoded polynucleotides, each of the components including parallel variable width microchannels.

In some embodiments, a tissue section has been permeabilized. In some embodiments, a tissue section was frozen prior to being permeabilized.

In some embodiments, a tissue section is mounted on a microscope slide.

In some embodiments, a processed tissued (e.g., a FFPE tissue) is mammalian tissue, optionally human tissue. In some embodiments, a processed tissue is a bacterial tissue.

In some embodiments, each of a first component and a second component comprises 5-1000 variable width microchannels, each of the microchannels having (i) an inlet port and an outlet port, (ii) a width of 2-150 μm, at the inlet port and the outlet port, and (iii) a width of 2-50 μm at the tissue section.

In some embodiments, a first component and a second component are oriented at an angle of greater than 10 degrees relative to each other during delivery of a first set of barcoded polynucleotides and a second set of barcoded polynucleotides.

In some embodiments, a first component and a second component are oriented perpendicular relative to each other during delivery of a first set of barcoded polynucleotides and a second set of barcoded polynucleotides.

In some embodiments, imaging is with an optical microscope or a fluorescence microscope.

In some embodiments, mapping comprises: (i) calculating gene expression levels based on sequencing reads; (ii) constructing a spatial molecular expression map by correlating gene expression levels to spatial sequences within the sequencing reads; and (iii) correlating the spatial molecular expression map to the sample image. In some embodiments, calculating gene expression levels comprises aligning sequencing reads to a reference genome. In some embodiments, a reference genome is derived from a mammalian genome. In some embodiments, a mammalian genome is a human genome or a rodent genome. In some embodiments, constructing a spatial molecular expression map comprises generating a uniform manifold approximation and projection map (UMAP). In some embodiments, step (iii) further comprises correlating spatial sequences within the sequencing reads to locations within the sample image.

In some embodiments, a first set of barcoded polynucleotides comprises a sequence having 90% sequence identity to any one of SEQ ID NOs: 1-50. In some embodiments, a first set of barcoded polynucleotides comprises a sequence according to any one of SEQ ID NOs: 1-50. In some embodiments, a second set of barcoded polynucleotides comprises a sequence having 90% sequence identity to any one of SEQ ID NOs: 51-100. In some embodiments, a second set of barcoded polynucleotides comprises a sequence according to any one of SEQ ID NOs: 51-100.

### BRIEF DESCRIPTION OF THE DRAWINGS

FIGS. 1A-1J show Patho-DBIT workflow, technical performance, and spatial mapping of mouse embryo. (FIGS. 1A-1B) Schematic workflow, molecular underpinnings, and technological spectrum of Patho-DBIT. Three major steps include (1) formalin-fixed paraffin-embedded (FFPE) tissue de-paraffinization and de-crosslink, (2) Enzymatic in situ polyadenylation and reverse transcription, (3) Spatial barcoding using a pair of microfluidic devices. Patho-DBiT utilizes poly(A) polymerase to add poly(A) tails to both A-tailed intact mRNA and non-A-tailed RNAs, enabling spatial characterization of molecules across the entire transcription process. Patho-DBiT demonstrates spatial profiling of high-sensitivity transcriptome, alternative splicing, variations printed in pre-RNAs, microRNAs, and RNA dynamics. The schematic flows left (FIG. 1A) to right (FIG. 1B) as indicated by the rightward arrow. (FIG. 1C) Patho-DBiT's performance and versatility on an E13 mouse embryo FFPE section. Top left: H&E staining of an adjacent section. The square indicates the region of interest (ROI). Top right: tissue scanning post 50 μm-microfluidic device barcoding. Bottom: unsupervised clustering identified 20 transcriptomic clusters, closely aligning with the H&E tissue histology. (FIG. 1D) Spatial pan-mRNA and UMI count maps. (FIG. 1E) Correlation analysis between replicates shows the high reproducibility of Patho-DBIT. Pearson correlation coefficient is indicated. (FIG. 1F) Read coverage along the gene body from 5′ to 3′ and the percentage of reads mapped to the 5′ UTR. Comparison involves two Patho-DBiT replicates with normal DBiT mapping without polyadenylation. (FIG. 1G) Comparison of the proportion of mapped RNA categories between Patho-DBIT and normal DBiT. Patho-DBiT demonstrates a similarly low level of mapped rRNA percentage compared to normal DBiT. (FIG. 1H) Integration of spatial RNA data with scRNA-seq mouse organogenesis data (Cao et al., Nature 2019). (FIGS. 1I and 1J) Distribution of gene and UMI counts in different tissue types at varying spatial resolutions. Patho-DBIT is benchmarked against another sequencing-based spatial technology, Visium from 10× Genomics on both FFPE and fresh frozen tissues.

FIGS. 2A-2K show spatial co-mapping of alternative splicing and gene expression in the mouse brain. (FIG. 2A) Patho-DBiT profiling of an adult mouse C57BL/6 FFPE brain section. Left: H&E staining of an adjacent section. Middle: tissue scanning of the region of interest (ROI) post 50 μm-microfluidic device barcoding. Right: spatial pan-mRNA and UMI count maps. (FIG. 2B) Unsupervised clustering identified 15 transcriptomically distinct clusters, and their distribution closely aligned with the region annotation of a corresponding coronal section from the Allen Mouse Brain Atlas (section 89, P56). (FIG. 2C) Integration of spatial RNA data with single-cell transcriptomics from cells in the mouse cortex and hippocampus (Yao et al., Cell 2021). (FIG. 2D) Molecular underpinnings of alternative splicing detection by Patho-DBIT. (FIG. 2E) Number of significant differentially spliced events and corresponding parental genes between each pair of two regions of the mouse brain. A splicing event is deemed significant if it exhibits an exon inclusion level difference >0.05 between two regions, with a false discovery rate (FDR) of ≤0.05. (FIG. 2F) Dot plot showing the top-ranked 12 genes exhibiting significant regional differences in exon inclusion levels. Gene dot size corresponds to the percentage of pixels expressing the gene, while isoform dot size indicates the percentage of junction reads derived from the inclusion/skipping isoform over both isoforms. The shade reflects the normalized expression level of each gene or isoform. (FIG. 2G and FIG. 2H) Junction read coverage of My16 (FIG. 2G) and Ppp3ca (FIG. 2H) splicing event in specific brain regions. Spatial expression patterns of the gene, exon inclusion isoform, and exon skipping isoform are shown. (FIG. 2I) Left: spatial variations in A-to-I RNA editing in the mouse brain. Right: distribution of editing ratio across all editing sites and the expression level of ADAR-encoding genes (Adarb1 and Adarb2) in different brain regions. Box whiskers show the minimum and maximum values. The dot size indicates the percentage of pixels expressing the gene, and the shade represents normalized expression level. (FIG. 2J) Left: spatial Adarb1 expression. Right: correlation between the Adarb1 expression and the average reginal editing ratio across various brain regions. Spearman correlation coefficient=0.89, p-value=0.012. (FIG. 2K) Correlation between regional editing ratios detected by short-read Illumina sequencing-based Patho-DBIT and those detected by long-read Nanopore sequencing, as reported in the reference literature (Lebrigand et al., Nucleic Acids Research 2023). Analysis centered on 259 editing sites detected by both technologies, revealing a robust Pearson correlation coefficient of 0.86 (p-value <2.2c-16).

FIGS. 3A-3I show high-sensitivity spatial transcriptomics of a AITL sample stored for five years. (FIG. 3A) Spatial transcriptome mapping of a subcutaneous nodule section from a patient diagnosed with AITL. The FFPE block has been stored at room temperature for five years before the Patho-DBiT assay. Left top: H&E staining of an adjacent section. Left bottom: tissue scanning post 50 μm-microfluidic device barcoding. Right: unsupervised clustering revealed 10 distinct clusters, aligning closely with the H&E tissue histology. (FIG. 3B) Heatmap showing top ranked DEGs defining each cluster. (FIG. 3C) Spatial phenotyping of an adjacent section using the CODEX technology (Co-Detection by Indexing). White square indicates the region of interest (ROI) in FIG. 3A. (FIG. 3D) Spatial distributions of B cells, T cells, and macrophages revealed by Patho-DBIT, exhibiting a strong Pearson correlation with the proteomic data generated from CODEX. Genes defining each module score are listed. (FIG. 3E) Top: CODEX data from the yellow square indicated area in FIG. 3C showing active expression of B cell marker (CD20), T follicular helper cell (Tfh) marker (CD4), and follicular dendritic cell marker (CD21). Bottom: Volcano plot of DEGs in Cluster 0 corresponding to the indicated region. (FIGS. 3F-3G) Ligand-receptor interactions within Cluster 0. The distinctive communication pattern between CXCL13 and its receptor genes (CXCR3, CXCR4, and CXCR5) is indicated. Edge thickness is proportional to correlation weights. (FIG. 3H) Corresponding canonical signaling pathways regulated by the DEGs in Cluster 0. z score is computed and used to reflect the predicted activation level (z>0, activated; z<0, inhibited; z>2 or z≤-2 can be considered significant). (FIG. 31) Graphical network of canonical pathways, upstream regulators, and biological functions regulated by DEGs identified in Cluster 0.

FIGS. 4A-4H show Patho-DBIT enables spatial variation profiling for tumor discrimination. (FIG. 4A) Spatial transcriptome mapping of a gastric antrum biopsy section from a patient diagnosed with extranodal marginal zone lymphoma of mucosa-associated lymphoid tissue (MALT). The FFPE block was stored at room temperature for three years. Left top: tissue scanning with region of interest (ROI) indicated with square. Left bottom: H&E staining of an adjacent section. Right: unsupervised clustering revealed 9 distinct clusters, aligning closely with the H&E tissue histology. (FIG. 4B) Spatial identification of representative cell types through curated expression of canonical genes. Genes defining each module score are listed. (FIG. 4C) Patho-DBiT's ability to capture rare cell types in specific regions was cross-validated through immunofluorescence (IF). The IF staining of plasma cell marker (CD138) and macrophage marker (CD68) in the selected Region P and Region M in FIG. 4B was shown. (FIG. 4D) Molecular underpinnings of detecting variations printed in pre-mRNA by Patho-DBIT. (FIG. 4E) Comparison of genomic location coverage bandwidth between Patho-DBIT and other technologies. (FIG. 4F) Spatial expression map of accumulated single nucleotide variants (SNVs) burden. (FIG. 4G) Immunohistochemistry (IHC) staining of canonical markers commonly detected in MALT tumor cells (BCL-2 and CD43) on adjacent sections. (FIG. 4H) Unsupervised clustering of the spatial mutational SNV matrix. Left: Veen plot showing the pixel overlap between gene cluster E1 and SNV clusters M1 and M3. Right: genome-wide distribution of somatic variations in clusters M1 and M3 using pixels from the other clusters as controls. Only high-confidence variant loci were preserved for downstream analysis and visualization.

FIGS. 5A-5H show spatial microRNA-mRNA regulatory network in the MALT section. (FIG. 5A) MicroRNAs detected by Patho-DBiT in the MALT section, with the count of mapped reads peaking at 22 nucleotides. The pie chart illustrates the percentage distribution of the detected count number per spatial pixel. (FIG. 5B) Spatial distribution of the Smooth muscle cell Score. Genes defining this module score are listed. (FIG. 5C) Spatial mapping of smooth muscle cell specific miR-143 and miR-145. The read coverage mapped to the reference genome location, expression proportion in each identified cluster, and spatial distribution are shown. (FIG. 5D) Volcano plot showing differentially expressed microRNAs between the tumor and non-tumor regions. (FIGS. 5E-5F) Regulatory network between the top 20 upregulated microRNAs and the gene expression in the tumor region. Genes with the highest rankings, demonstrating positive or negative correlations with the microRNAs, were separately illustrated. Edge thickness is proportional to correlation weights. (FIG. 5G) Spatial expression map of the oncomiR miR-21. This microRNA significantly regulates 760 genes (Pearson R >0.1 or <−0.1, p-value <0.05). Cancer-related genes are defined based on the IPA data base. (FIG. 5H) Spatial expression map of the B cell lymphoma specific miR-155. Top: read coverage mapped to the reference genome location. Bottom left: spatial distribution. Bottom right: expression comparison between tumor and non-tumor regions. Box whiskers show the minimum and maximum values. Significance level was calculated with two-tailed Mann-Whitney test, **** P <0.0001. (FIGS. 51-5J) Spatial interactions involving mir-155 and its upstream and downstream signaling pathways. Top 5 genes defining each module score are listed. The Pearson correlation between mir-155 expression and both signaling pathways was calculated across 447 spatial pixels within the tumor region.

FIGS. 6A-6H show tumor differentiation trajectory revealed by spatial RNA splicing dynamics. (FIG. 6A) Distribution of detected gene/UMI counts per spatial pixel from reads mapped to exonic or intronic region. The dashed lines indicate average level of gene or UMI count in the MALT section. (FIG. 6B) Unsupervised clustering of the combined exonic and intronic expression matrix. The analysis identified 14 clusters, showcasing UMAP visualization and featured expression of the B cell Score in clusters C3, C4, and C6. Genes defining this module score are listed. (FIG. 6C) Top: cell cycle score indicated by the S or G2/M stage. Bottom: IHC staining for Ki67 in the tumor region of an adjacent section. (FIG. 6D) Velocities derived from the dynamical RNA splicing activities are visualized as streamlines in a UMAP-based embedding. The coherence of the velocity vector field provides a measure of confidence, and the spatial velocity pattern within the tumor B cells region is highlighted. (FIG. 6E) Phase portraits showing the ratio of unspliced and spliced RNA for top-ranked genes driving the dynamic flow from cluster C4 to C6, along with their expression and velocity level within the three tumor clusters. The dashed line corresponds to the estimated splicing steady state. Positive velocity signifies up-regulation of a gene, observed when cells exhibit a higher abundance of unspliced mRNA for that gene than expected in steady state. Conversely, negative velocity indicates down-regulation of the gene. (FIG. 6F) Spatial pseudotime of underlying cellular processes based on the transcriptional dynamics. A discernible change is evident exclusively within the three tumor clusters, where a higher pseudotime number denotes a later differentiation stage. (FIG. 6G) Volcano plot showing DEGs between cluster C6 and C3. Signature large and small RNAs associated with increased dynamic activities are spatially visualized. (FIG. 6H) Correlation matrices of the signature RNAs evaluated in G. Only significant correlations (p-value <0.05) are represented as dots. Pearson's correlation coefficients from comparisons of RNA expression across pixels in the tumor region are visualized by intensity.

FIGS. 7A-7L show cellular level spatial mapping of a DLBCL section elucidates tumor progression. (FIG. 7A) Spatial transcriptome mapping of fundus nodule biopsy sections collected from the same patient depicted in FIG. 4A at the same time. The diagnosis progressed from low-grade MALT to DLBCL in this subsequent biopsy. Left: sections from two different regions underwent 10 μm-microfluidic device spatial barcoding. Right top: unsupervised clustering of Region 1 identified two clusters. Right bottom: unsupervised clustering of Region 2 revealed 10 transcriptomically distinct subpopulations. (FIG. 7B) Spatial characterization of representative cell types based on the expression of signature gene. Genes defining each module score are listed. (FIG. 7C) Spatial heterogencities and interactions among tumor B cells. Left top: comparative analysis of chemokine gene expression between clusters 2 and 5. Left bottom: signaling pathways regulated by DEGs between cluster 2 vs. cluster 5. Right: spatial distribution of the Chemokine Score and RhoA Signaling Score. Genes defining each module score are listed. (FIG. 7D) Cellular-level spatial mapping unveils a distinct transcriptomic neighborhood. Left: comparative analysis of gastric mucus-secreting cell related gene expression between clusters 4, 7, and 8. Right top: enlarged transcriptomic neighborhood highlighted by white square in FIG. 7A. Right bottom: tissue morphology of the corresponding area defined by H&E staining of an adjacent section. (FIG. 7E) Spatial analysis elucidates the molecular dynamics driving tumor progression. Left: schematic illustration showing comparative analysis. Right: signaling pathways regulated by DEGs between tumor B cells in DLBCL vs. MALT biopsy, revealing a significant upregulation of NF-κB signaling and its associated upstream and downstream pathways. (FIG. 7F) Expression comparison of key genes involved in the NF-κB signaling between DLBCL vs. MALT biopsy. (FIG. 7G) IHC staining for Ki67 on adjacent sections from the two biopsies. (FIG. 7H) Spatial expression mapping of genes encoding plasma cell kappa and lambda chains in the two biopsies. (FIG. 7I) ISH staining for kappa and lambda chain mRNA in the designated area in FIG. 7H. (FIG. 7J)Distance distribution between macrophages and tumor B cells in the two biopsies. Significance level was calculated with two-tailed Mann-Whitney test, **** P<0.0001. (FIG. 7K) Signaling pathways regulated by DEGs between macrophages in DLBCL vs. MALT biopsy, revealing a significant upregulation of macrophage alternative activation signaling and its associated pathways. (FIG. 7L) Ligand-receptor interactions between macrophage cluster 1 and tumor B cell clusters 2 and 5. The distinctive communication pattern of TGF-β (TGFB1) and the integrin family (ITGB1, ITGB5, and ITGB8) is indicated and spatially visualized. Edge thickness is proportional to correlation weights. In FIGS. 7C, 7E, and 7K, z score is computed and used to reflect the predicted activation level (z>0, activated; z<0, inhibited; z>2 or z≤-2 can be considered significant).

### DETAILED DESCRIPTION

Spatial transcriptomics is revolutionizing the field's understanding of developmental biology, oncology, and disease pathology, mapping intricate gene expression patterns within their native tissue context. This innovation is instrumental in discerning subtle nuances of transcriptional diversity, cellular signaling, and microscopic niches within biological tissues, promising to refine diagnostic precision and guide the creation of targeted treatment modalities. To date, however, the scope of the field primarily revolves around the analysis of messenger RNA (mRNA) expression. The transcriptome in eukaryotic cells is a dynamic reflection of all RNA molecules encompassing not only mRNA that dictates protein production but also small RNAs, spliced variants, and other non-coding RNAs with regulatory functions. Thus, spatial profiling of different RNA species throughout the life cycle is imperative for the accurate analysis of RNA biology in complex tissues.

Formalin-fixed paraffin-embedded (FFPE) tissues are essential in clinical practice, routinely used in pathology diagnostics. Serving as the conventional method for preserving surgical pathology samples, FFPE processing maintains tissue morphology and cellular integrity at room temperature and is more economical than fresh frozen specimens when considering storage, space, and personnel costs. Pathology departments have accrued vast collections of FFPE blocks over time, creating a rich, yet underutilized, compendium of materials that, accompanied by comprehensive clinical data, stands as a treasure trove for translational research.

Nevertheless, FFPE specimens pose certain challenges. The RNA within these samples is susceptible to degradation during the paraffin-embedding process and can further experience heightened degradation under suboptimal storage conditions. Additionally, RNA can undergo chemical modifications, resulting in fragmentation or resistance to the enzymatic reactions required for sequencing. The loss of poly(A) tails introduces another layer of complexity, restricting the use of oligo-dT primed reverse transcription. Consequently, options for spatially profiling RNA molecules in this challenging tissue type are limited. While imaging-based platforms like MERSCOPE (Vizgen), CosMx (Nanostring), and Xenium (10× Genomics) offer subcellular-level resolution, their capacity is constrained, mapping only up to thousands of mRNAs combined with a panel of surface proteins. Similarly, the Visium (10× Genomics) chemistry for FFPE samples relies on a predefined panel to target and capture RNA fragments, enabling nearly transcriptome-level profiling through next-generation sequencing, yet still confined to protein-coding genes.

In this evolving landscape, the present disclosure presents Patho-DBIT, an innovative technology tailored, for example, for spatial whole transcriptome sequencing meticulously crafted to address the distinctive challenges of processed tissue samples such as clinically archived FFPE tissues. Patho-DBIT, in some embodiments, integrates in situ polyadenylation, deterministic barcoding in tissue using microfluidic chips, and computational innovations to navigate and decode the rich RNA biology inherent in FFPE samples. The methods described herein, in some aspects, adeptly capitalizes on RNA fragmentation, exploits the inhibitory effect against endogenous endonuclease activity, and appends poly(A) tails to a broad spectrum of RNA species, thereby overcoming traditional barriers associated with processed tissue (e.g., FFPE) samples. By spatially barcoding A-tailed intact mRNAs, fragmented mRNAs lacking 3′ ends, various forms of large and small non-coding RNAs, splicing isoforms, and precursor RNAs carrying genetic mutations, the methods provided herein enable a deeper appreciation of high-sensitivity transcriptomics, alternative splicing, variation profile, microRNA-mRNA regulation, and RNA dynamics within complex tissues. Patho-DBIT represents a powerful technology for exploring spatial RNA biology in processed (e.g., FFPE) tissues, in some aspects, promising valuable insights into inter alia human disease development and biomarker discovery beyond gene expression.

### Polyadenylation of RNA Species

Aspects of the present disclosure relate to polyadenylation of RNA molecules that lack a poly(A) tail (e.g., that naturally lack a poly(A) tail or lack a poly(A) tail as a result of fragmentation during tissue processing and/or storage). Fragmented RNA includes RNA molecules that have degraded over time or have been damaged, for example, as a result of exposure to tissue fixation agents and/or processes (e.g., dehydration, clearing, and/or infiltration) and/or freeze/thaw cycling. Clinicians often collect tissue samples from patients (subjects) post-mortem for pathological analyses. Many pathological analyses require the tissue samples to be cut into smaller tissue sections and fixed in formalin and paraffin. Tissue sections fixed in formalin and paraffin are referred to herein as formalin-fixed paraffin-embedded (FFPE) tissue sections. At the time of initial fixing, nucleic acid molecules within the tissue have similar integrity as compared to the integrity of the nucleic acid molecules prior to fixing. After fixing, however, FFPE tissue sections are often archived for years, even decades, in temperatures as low as −80° C., which results in damage to nucleic acid molecules within the tissue sections. Accordingly, an abundance of genomic information within FFPE tissue sections is undetectable by current methods of analysis because the nucleic acids are damaged and fragmented. Fragmented RNA molecules represent an abundance of information that is inaccessible without improved methods of detection.

Tissue processing is a technique by which fixed tissues are made suitable for embedding within a supportive medium such as paraffin, and typically includes three sequential steps: dehydration, clearing, and infiltration. During tissue processing, water is removed from cells and replaced with a medium that solidifies, allowing thin sections to be cut, for example, on a microtome. While the present disclosure refers to FFPE tissue sections throughout, the methods herein can be applied to other tissue sections, including other processed tissue sections including, for example, those in which RNA is fragmented due to processing methods.

To access the abundance of information within FFPE tissue sections and other processed tissue sections, the inventors of the present disclosure developed a method of modifying fragmented RNA molecules within these tissue sections for genomic analysis. Current genomic analysis techniques rely on the naturally occurring poly(A) tail found on messenger RNA (mRNA) molecules. The scientific study of transcriptomics is founded in the understanding that gene transcripts (mRNA molecules) naturally contain a poly(A) tail, which can be used for molecule capture and sequencing. Recognizing that these processes fail to capture a broad spectrum of important RNA species, the inventors have combined in situ polyadenylation with deterministic barcoding to create a pool of polyadenylated RNA molecules, including intact mRNAs, fragmented mRNAs, various forms of large and small non-coding RNAs, splicing isoforms, and precursor RNAs carrying single nucleotide variations, within a processed tissue section (e.g., an FFPE tissue section) for downstream spatial omics analyses.

Polyadenylation is the addition of a poly(A) tail to an RNA molecule. A poly(A) tail includes a stretch of adenosine monophosphates, important for nuclear export, translation, and RNA stability. The length of a poly(A) sequence can vary. For example, poly(A) sequence can have a length of 5 to 50 nucleotides (e.g., 5 to 40, 5 to 30, 5 to 20, 5 to 10, 10 to 50, 10 to 40, 10 to 30, or 10 to 20 nucleotides). In some embodiments, poly(A) sequence has a length of 5, 10, 15, 20, 25, 30, 35, 40, 45, or 50 nucleotides. Longer poly(A) sequences are contemplated herein.

In genomic sequencing, the poly(A) tail is used to enrich for RNA molecules of interest. This process of poly(A) selection is accomplished by exposing a sample to poly(T) oligomers that capture poly(A) tails for additional analyses. Accordingly, a method of polyadenylating fragmented RNA molecules and RNA molecules that naturally lack a poly(A) tail facilitates capture of additional RNA molecules for downstream analysis. A poly(T) includes contiguous sequence of thymine (T) residues. The length of a poly(T) sequence can vary. For example, a poly(T) sequence can have a length of 5 to 50 nucleotides (e.g., 5 to 40, 5 to 30, 5 to 20, 5 to 10, 10 to 50, 10 to 40, 10 to 30, or 10 to 20 nucleotides). In some embodiments, a poly(T) sequence has a length of 5, 10, 15, 20, 25, 30, 35, 40, 45, or 50 nucleotides. Longer poly(T) sequences are contemplated herein.

While mRNA molecules are the most abundant polyadenylated RNA molecules, tissues sections include a broad spectrum of RNA species, such as intact mRNAs, fragmented mRNAs (described elsewhere herein), various forms of large and small non-coding RNAs, splicing isoforms, and precursor RNAs carrying single nucleotide variations (SNVs), none of which is naturally polyadenylated. Other non-limiting examples of RNA species that can be assessed using the methods of the disclosure include transfer RNA (tRNA) molecules, microRNA (miRNA) molecules, and ribosomal RNA (rRNA) molecules.

In some embodiments, a method comprises producing cDNA from polyadenylated fragmented RNAs. Polyadenylated fragmented RNAs are fragmented RNAs (relative to naturally occurring RNA) that have been modified to includes a poly(A) tail. Such polyadenylated fragmented species are considered to have been artificially (not naturally) polyadenylated. Thus, in some embodiments, fragmented RNA molecules are polyadenylated (artificially polyadenylated) following exposure to a polyadenylate polymerase. In some embodiments, RNA molecules that naturally lack a poly(A) tail, such as small non-coding RNA molecules and ribosomal RNA, are polyadenylated following exposure to a polyadenylate polymerase.

Polyadenylation can be accomplished, in some embodiments, by delivering a polyadenylate polymerase to a tissue section. A polyadenylate polymerase (PAP), also referred to as a poly(A) polymerase, is an enzyme involved in the formation of the poly(A) tail of the 3′ end of an RNA (catalyzing the addition of AMP from ATP to the 3′ hydroxyl of RNA). In some embodiments, a poly(A) polymerase is from Escherichia coli (E. coli). In some embodiments, a poly(A) polymerase is from yeast. In some embodiments, a method further comprises delivering a polyadenylation reagent to a tissue section. Polyadenylation reagents include poly(A) polymerase and other reagents involved in the formation of the poly(A) tail. Non-limiting examples of polyadenylation reagents include polyadenylation specificity factors, cleavage stimulation factors, cleavage factors, and polyadenylate binding proteins. Polyadenylation specificity factors, also referred to as cleavage/polyadenylation specificity factors, include, for example, CPSF. Cleavage stimulation factors include, for example, CstF. Cleavage factors include, for example, CFI and CFII. Polyadenylate binding proteins include, for example, PABII. Other polyadenylation reagents include AMP (adenosine monophosphate), ATP (adenosine triphosphate), reaction buffer, RNase inhibitor, and EDTA.

In nature, precursor mRNA bound to an RNA polymerase II is polyadenylated by a multi-protein complex that cleaves the 3′-most part of a newly synthesized RNA molecule and polyadenylates the end produced by this cleavage. CPSF catalyzes the initial 3′ cleavage reaction. CstF and CFI provide additional RNA-specificity to the multi-protein complex by binding to sites on the RNA molecule independent of the CPSF-binding site. CstF further signals for the newly synthesized RNA molecule to detach from RNA polymerase II. Through an unknown mechanism, CFII catalyzes additional cleavage reactions. Once an RNA molecule is cleaved, poly(A) polymerase builds a poly(A) tail on the RNA molecule by adding AMP units from ATP to the RNA, cleaving off pyrophosphate. PABII binds to the newly added, short poly(A) tail and increases the affinity of poly(A) polymerase to bind to the RNA. When the poly(A) tail reaches a specific length, CPSF activity is inhibited, and polyadenylation stops.

In some embodiments, polyadenylation of RNA molecules is accomplished by delivering poly(A) polymerase to a tissue section in the absence of additional polyadenylation enzymes. Poly(A) polymerase can catalyze the addition of a poly(A) tail to fragmented RNA molecules and RNA molecules naturally lacking a poly(A) tail without cleavage of the 3′-end of the RNA molecule. In some embodiments, a poly(A) polymerase and one or more cleavage factors are delivered to a tissue section to initiate cleavage of RNA molecules and subsequent polyadenylation of the RNA molecules. Delivering one or more cleavage factors to a tissue section in addition to poly(A) polymerase can increase polyadenylation specificity and efficiency. In some embodiments, a poly(A) polymerase a PABII are delivered to a tissue section to initiate polyadenylation of RNA molecules. Delivering PABII to a tissue section in addition to poly(A) polymerase can further increase polyadenylation specificity and efficiency. In some embodiments, a poly(A) polymerase, one or more cleavage factors, and a PABII are delivered to a tissue section to initiate cleavage of RNA molecules and subsequent polyadenylation of the RNA molecules. Delivering one or more cleavage factors and PABII to a tissue section in addition to poly(A) polymerase can further increase polyadenylation specificity and efficiency.

Aspects of the present disclosure relate to adding a nucleotide homopolymer to available 3′ ends of RNA molecules within a tissue section. A nucleotide homopolymer is a stretch of repeating nucleotides, for example, adenosine monophosphate (dAMP), guanosine monophosphate (dGMP), thymidine monophosphate (dTMP), or cytidine monophosphate (dCMP). A stretch of repeating dAMPs is referred to as a poly(A) tail. A stretch of repeating dGMPs is referred to as a poly(G) tail. A stretch of repeating dTMPs is referred to as a poly(T) tail. A stretch of repeating dCMPs is referred to as a poly(C) tail. The length of a poly(A) sequence can vary. For example, poly(A) sequence can have a length of 5 to 50 nucleotides (e.g., 5 to 40, 5 to 30, 5 to 20, 5 to 10, 10 to 50, 10 to 40, 10 to 30, or 10 to 20 nucleotides). In some embodiments, poly(A) sequence has a length of 5, 10, 15, 20, 25, 30, 35, 40, 45, or 50 nucleotides. Longer poly(A) sequences are contemplated herein. The length of a poly(G) sequence can vary. For example, poly(G) sequence can have a length of 5 to 50 nucleotides (e.g., 5 to 40, 5 to 30, 5 to 20, 5 to 10, 10 to 50, 10 to 40, 10 to 30, or 10 to 20 nucleotides). In some embodiments, poly(G) sequence has a length of 5, 10, 15, 20, 25, 30, 35, 40, 45, or 50 nucleotides. Longer poly(G) sequences are contemplated herein. The length of a poly(T) sequence can vary. For example, poly(T) sequence can have a length of 5 to 50 nucleotides (e.g., 5 to 40, 5 to 30, 5 to 20, 5 to 10, 10 to 50, 10 to 40, 10 to 30, or 10 to 20 nucleotides). In some embodiments, poly(T) sequence has a length of 5, 10, 15, 20, 25, 30, 35, 40, 45, or 50 nucleotides. Longer poly(T) sequences are contemplated herein. The length of a poly(C) sequence can vary. For example, poly(C) sequence can have a length of 5 to 50 nucleotides (e.g., 5 to 40, 5 to 30, 5 to 20, 5 to 10, 10 to 50, 10 to 40, 10 to 30, or 10 to 20 nucleotides). In some embodiments, poly(A) sequence has a length of 5, 10, 15, 20, 25, 30, 35, 40, 45, or 50 nucleotides. Longer poly(C) sequences are contemplated herein.

RNA molecules, including fragmented RNA molecules and RNA molecules naturally lacking a poly(A) tail can be extracted from a sample (e.g., a tissue section) by adding a poly(A) tail to the 3′ ends the RNA molecules within a tissue section and exposing the sample to poly(T) oligomers that capture poly(A) tails. A poly(T) includes contiguous sequence of thymine (T) residues. RNA molecules, including fragmented RNA molecules and RNA molecules naturally lacking a poly(A) tail can be extracted from a sample (e.g., a tissue section) by adding a poly(G) tail to the 3′ ends the RNA molecules within a tissue section and exposing the sample to poly(C) oligomers that capture poly(G) tails. A poly(C) includes contiguous sequence of cytosine (C) residues. RNA molecules, including fragmented RNA molecules and RNA molecules naturally lacking a poly(A) tail can be extracted from a sample (e.g., a tissue section) by adding a poly(T) tail to the 3′ ends the RNA molecules within a tissue section and exposing the sample to poly(A) oligomers that capture poly(T) tails. A poly(A) includes contiguous sequence of adenosine (A) residues. RNA molecules, including fragmented RNA molecules and RNA molecules naturally lacking a poly(A) tail can be extracted from a sample (e.g., a tissue section) by adding a poly(C) tail to the 3′ ends the RNA molecules within a tissue section and exposing the sample to poly(G) oligomers that capture poly(C) tails. A poly(G) includes contiguous sequence of guanosine (G) residues.

A poly(N) sequence includes a poly(A) sequence, a poly(T) sequence, a poly(G) sequence, and/or a poly(C) sequence. Thus, “N” may be A, T, C, or G. Aspects of the present disclosure relate to the addition of a poly(N) (i.e., poly(A), poly(G), poly(T), or poly(C)) sequence to available 3′ ends of all RNA molecules within a tissue section. An RNA molecule comprising a poly(N) sequence can be extracted from a tissue section using a complementary poly(N) sequence. For example, an RNA comprising a poly(A) sequence can be extracted from a tissue section using a corresponding poly(T) sequence, an RNA comprising a poly(G) sequence can be extracted from a tissue section using a corresponding poly(C) sequence, an RNA comprising a poly(T) sequence can be extracted from a tissue section using a corresponding poly(A) sequence, and an RNA comprising a poly(C) sequence can be extracted from a tissue section using a corresponding poly(G) sequence.

Poly(N) sequences can be added to available 3′ ends of all RNA molecules within a tissue section by delivering to the tissue section a poly(N) polymerase. A poly(N) polymerase is a poly(A) polymerase, a poly(G) polymerase, a poly(T) polymerase, or a poly(C) polymerase. A poly(N)-tailed RNA molecule is an RNA molecule comprising a poly(N) tail added by the enzymatic activity of a poly(N) polymerase. A poly(N) polymerase acts by catalyzing the addition of dNMP from dNTP to the 3′hydroxyl of an RNA. In some embodiments, poly(A) polymerase acts by catalyzing the addition of dAMP from dATP to the 3′hydroxyl of an RNA. In some embodiments, poly(G) polymerase acts by catalyzing the addition of dGMP from dGTP to the 3′hydroxyl of an RNA. In some embodiments, poly(T) polymerase acts by catalyzing the addition of dTMP from dTTP to the 3′hydroxyl of an RNA. In some embodiments, poly(C) polymerase acts by catalyzing the addition of dCMP from dCTP to the 3′hydroxyl of an RNA.

Aspects of the present disclosure relate to adding to RNA molecules within a tissue section a poly(N) tail by delivering to the tissue section a poly(N) polymerase. In some embodiments, the RNA molecules are fragmented RNA molecules, RNA molecules that naturally lack a poly(A) tail or any RNA molecule within a tissue section with an available 3′ end. A 3′ end of an RNA molecule is available if a 3′hydroxyl is exposed or if the RNA molecule is capable of accepting a poly(N) tail. In some embodiments, the present disclosure relates to adding to RNA molecules within a tissue section a poly(A) tail by delivering to the tissue section a poly(A) polymerase. In some embodiments, the present disclosure relates to adding to RNA molecules within a tissue section a poly(G) tail by delivering to the tissue section a poly(G) polymerase. In some embodiments, the present disclosure relates to adding to RNA molecules within a tissue section a poly(T) tail by delivering to the tissue section a poly(T) polymerase. In some embodiments, the present disclosure relates to adding to RNA molecules within a tissue section a poly(C) tail by delivering to the tissue section a poly(C) polymerase.

An RNA molecule comprising a poly(N) tail added by the enzymatic activity of a poly(N) polymerase is referred to as a poly(N)-tailed RNA. A fragmented RNA molecule comprising a poly(N) tail added by the enzymatic activity of a poly(N) polymerase is referred to as a poly(N)-tailed fragmented RNA. An RNA molecule comprising a poly(A) tail added by the enzymatic activity of a poly(A) polymerase is referred to as a poly(A)-tailed RNA. A fragmented RNA molecule comprising a poly(A) tail added by the enzymatic activity of a poly(A) polymerase is referred to as a poly(A)-tailed fragmented RNA. An RNA molecule comprising a poly(G) tail added by the enzymatic activity of a poly(G) polymerase is referred to as a poly(G)-tailed RNA. A fragmented RNA molecule comprising a poly(G) tail added by the enzymatic activity of a poly(G) polymerase is referred to as a poly(G)-tailed fragmented RNA. An RNA molecule comprising a poly(T) tail added by the enzymatic activity of a poly(T) polymerase is referred to as a poly(T)-tailed RNA. A fragmented RNA molecule comprising a poly(T) tail added by the enzymatic activity of a poly(T) polymerase is referred to as a poly(T)-tailed fragmented RNA. An RNA molecule comprising a poly(C) tail added by the enzymatic activity of a poly(C) polymerase is referred to as a poly(C)-tailed RNA. A fragmented RNA molecule comprising a poly(C) tail added by the enzymatic activity of a poly(C) polymerase is referred to as a poly(C)-tailed fragmented RNA.

Spatially Barcoded Complementary DNA (cDNA)

Aspects of the disclosure relate to the production of spatially barcoded complementary deoxyribonucleic acids (cDNAs) from polyadenylated RNAs. While RNA can be the type of molecule of interest in some embodiments, it is typically converted to cDNA for downstream analyses. Thus, in some embodiments, an RNA molecule (e.g., a polyadenylated RNA molecule) is reverse transcribed into a cDNA molecule. Reverse transcription can be accomplished, for example, by delivering reverse transcription reagents to a tissue section (e.g., via a microfluidic device). Reverse transcription reagents can include one or more reagents selected from reverse transcriptases, reverse transcription primers, dNTPS, and RNase inhibitor. There are numerous commercially available reverse transcription reagents and kits available for use with the methods described herein. An exemplary reverse transcription reaction is described herein in the Examples.

Barcoded polynucleotides include a (one or more) short, distinct (e.g., unique) sequence of nucleotides, known as barcodes (also referred to herein as barcode sequences), used to identify the polynucleotide among other polynucleotides, for example in a tissue or reaction mixture. A unique molecular identifier (UMI) is an example of a barcode sequence. UMIs can be attached to individual biomolecules, such as DNA or RNA molecules, before they undergo amplification to uniquely label each molecule so it can be distinguished from others, even after amplification. In some embodiments, a cDNA includes a spatial barcode. Spatial barcoding extends the concept of polynucleotide barcoding to include spatial information about where specific molecules are located within a biological sample, such as a tissue section or a single cell. This approach enables not only the identification what molecules are present, but also a determination of their precise locations. Thus, a spatially barcoded cDNA comprises a barcode (e.g., formed from a combination of barcoded polynucleotides, such as a barcoded polynucleotide from a first set of barcoded polynucleotides and a barcoded polynucleotide from a second set of barcoded polynucleotides) that includes spatial information enabling identification of the location of the cDNA, within a tissue section, for example. See, e.g., Williams C G et al. Genome Medicine 2022; 14 (68): 1-18; Liszczak G et al. Angew Chem Int Ed Engl. 2019 Mar. 22; 58 (13): 4144-4162; and International Publication No. WO 2021/067246, Deterministic Barcoding for Spatial Omics Sequencing.

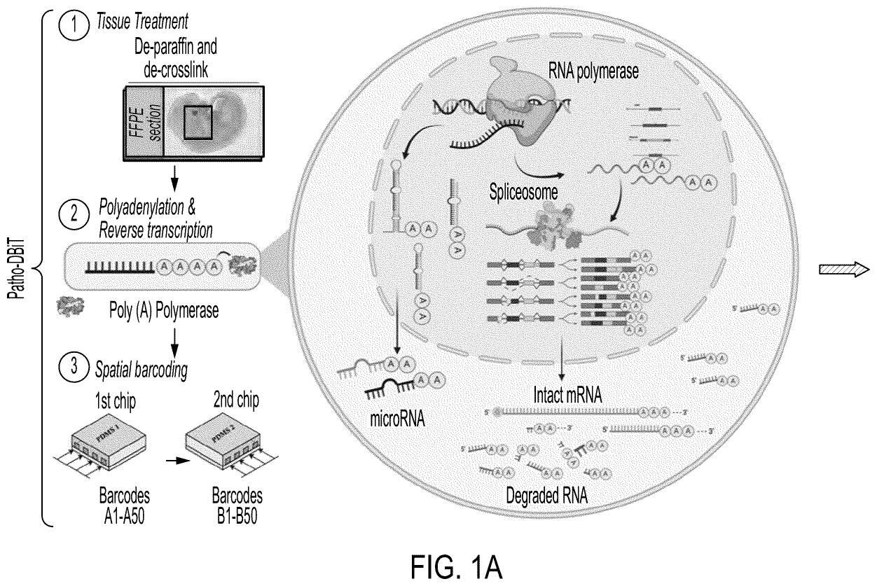

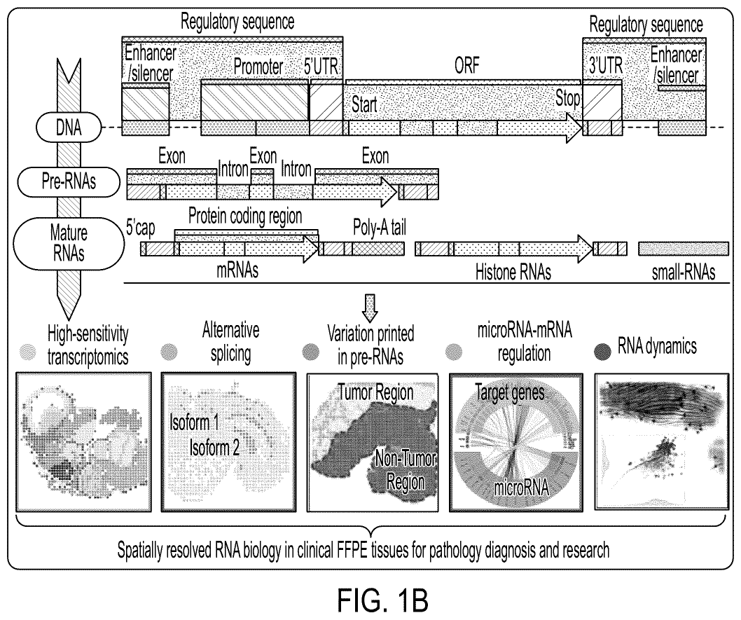

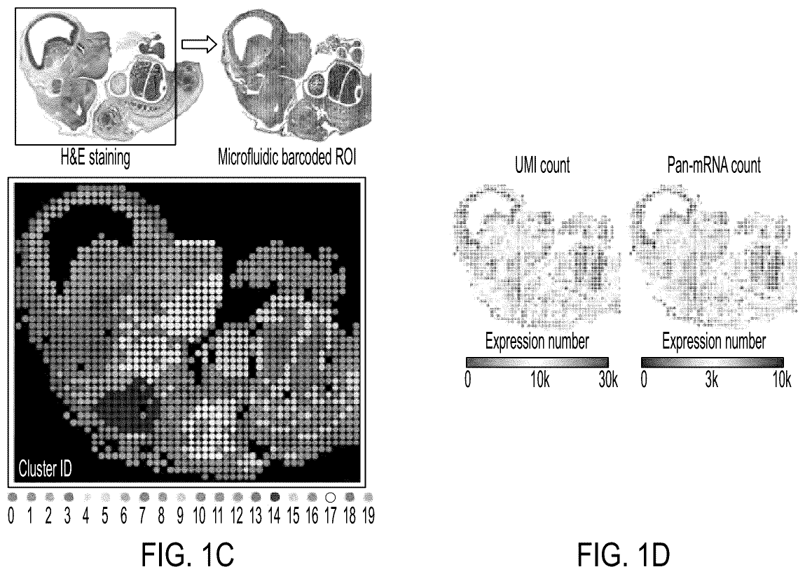

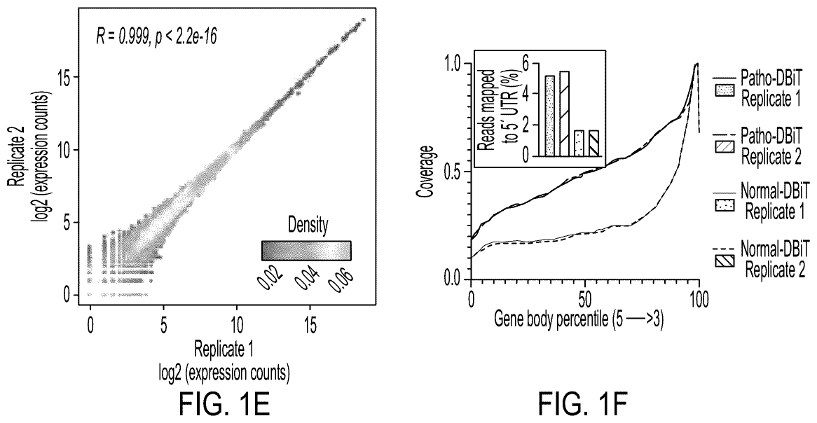

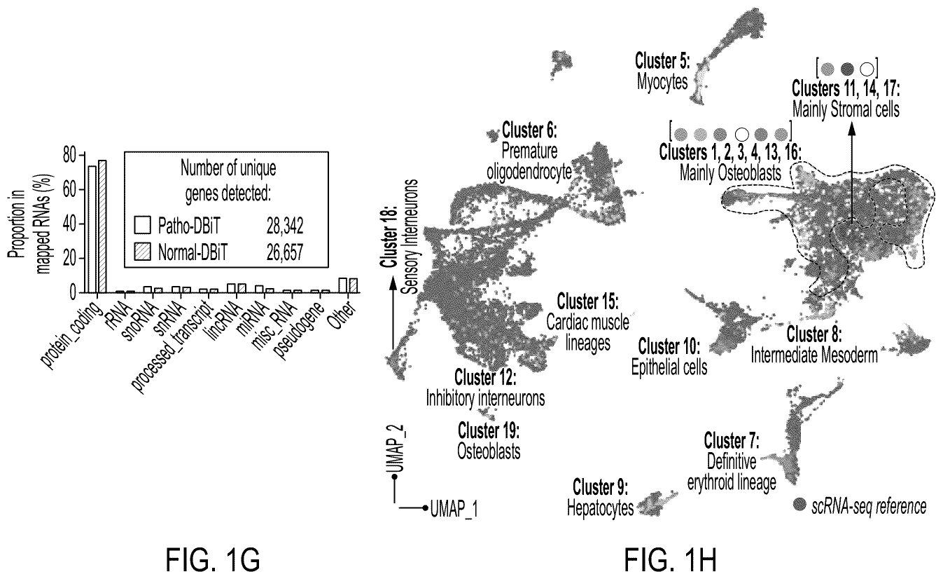

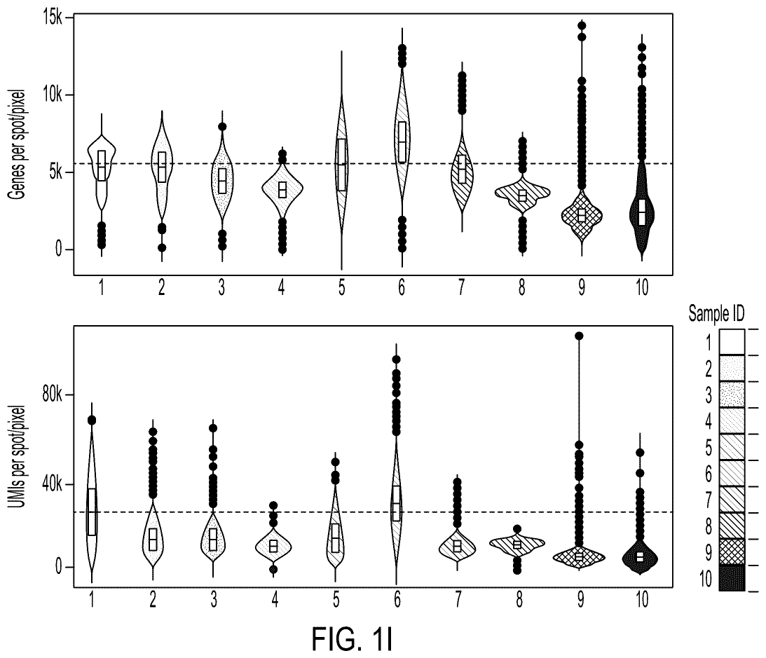

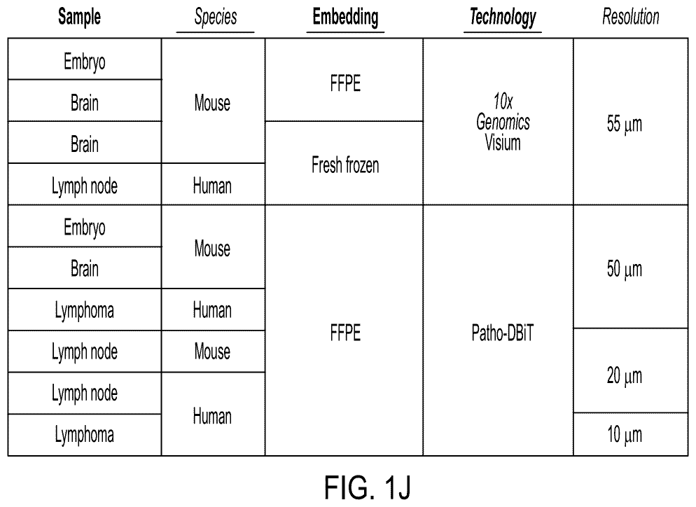

The term “unique” in the context of barcoding is with respect to the molecules in a single biological sample (e.g., tissue section) and includes only one of a particular molecule or subset of molecules in the sample. As an illustrative example, polynucleotides of subset A1 (of Barcode A) can be coded with a specific barcode sequence, while polynucleotides of subsets A2, A3, A4, etc. are each coded with a different barcode sequence, each barcode specific to the particular Barcode A subset. Likewise, polynucleotides of subset B1 (of Barcode B) can be coded with a specific barcode sequence, while the polynucleotides of subsets B2, B3, B4, etc. are each coded with a different barcode sequence, each barcode specific to the particular Barcode B subset. When the barcodes are delivered in a spatially-defined manner using, for example, a microfluidic device as described herein (see, e.g., FIG. 1A (3), left image), Barcodes of an A subset (e.g., A1, A2, A3, etc.) can provide spatial information along one axis (e.g., an X-axis), while Barcodes of a B subset (e.g., B1, B2, B3, etc.) can provide spatial information along another axis (e.g., an Y-axis) such that a single spatially barcoded polynucleotide (e.g., cDNA), appended with a Barcode A and a Barcode B, can be mapped to a specific location within a tissue section, identifiable by X (Barcode A) and Y (Barcode B) coordinates. Example sequences of barcoded polynucleotides are provided in Table 1.

The length of a barcode sequence can vary. For example, a barcode sequence can have a length of 5 to 50 nucleotides (e.g., 5 to 40, 5 to 30, 5 to 20, 5 to 10, 10 to 50, 10 to 40, 10 to 30, or 10 to 20 nucleotides). In some embodiments, a barcode has a length of 5, 10, 15, 20, 25, 30, 35, 40, 45, or 50 nucleotides. Longer barcode sequences are contemplated herein.

Methods herein can include delivering to a tissue section a first set of barcoded polynucleotides and a second set of barcoded polynucleotides. Any given set of barcoded polynucleotides can include any number of barcoded polynucleotides. In some embodiments, a set of barcoded polynucleotides includes 5 to 1000 barcoded polynucleotides. For example, a set of barcoded polynucleotides can comprise 5 to 900, 5 to 800, 5 to 700, 5 to 600, 5 to 500, 5 to 400, 5 to 300, 5 to 200, 5 100, 10 to 1000, 10 to 900, 10 to 800, 10 to 700, 10 to 600, 10 to 500, 10 to 400, 10 to 300, 10 to 200, 20 to 1000, 20 to 900, 20 to 800, 20 to 700, 20 to 600, 20 to 500, 20 to 400, 20 to 300, 20 to 200, 50 to 1000, 50 to 900, 50 to 800, 50 to 700, 50 to 600, 50 to 500, 50 to 400, 50 to 300, or 50 to 200 barcoded polynucleotides. In some embodiments, a set of barcoded polynucleotides includes more than 1000 barcoded polynucleotides.

Methods herein can include delivering to a tissue section ligation reagents to produce spatially barcoded cDNAs. Thus, in some embodiments, one or more barcoded polynucleotides is/are linked to a cDNA via ligation (i.e., is ligated to the cDNA). In some embodiments, cDNA molecules are reverse transcribed from polyadenylated RNA molecules, then one or more of the cDNA molecules is/are linked to one or more barcoded polynucleotides.

In some embodiments a barcoded polynucleotide comprises a PCR handle, which as is known in the art includes a constant sequence identical on a set of primers, for example, which allows PCR amplification. In some embodiments, a PCR handle end sequence comprises (e.g., is terminally functionalized with) biotin or another molecule that can be used for purification of PCR amplified polynucleotides.

### Tissue Samples

In some embodiments, a biological sample is a tissue sample. A tissue sample can be from adult tissue, embryonic tissue, or fetal tissue, for example. In some embodiments, a tissue sample is from a mammal, such as a human. Other tissues from which a tissue sample can be obtained include be murine (e.g., mouse or rat), feline (e.g., cat), canine (e.g., dog), equine (e.g., horse), bovine (e.g., cow), leporine (e.g., rabbit), porcine (e.g., pig), hircine (e.g., goat), ursine (e.g., bear), or piscine (e.g., fish) species. In some embodiments, a tissue sample is a human tissue sample.

In some embodiments, a tissue sample is fixed, and thus is referred to as a fixed tissue. Fixation (e.g., tissue fixation) refers to the process of chemically preserving the natural state of a sample, for example, for subsequent histological analysis. Various fixation agents are routinely used, including, for example, formalin (e.g., formalin fixed paraffin embedded tissue), formaldehyde, paraformaldehyde and glutaraldehyde, any of which can be used herein to fix a biological sample. In some embodiments, a fixed tissue is formalin-fixed paraffin-embedded (FFPE) tissue. In some embodiments, a fixation process involves perfusion of the animal from which the sample is collected. In some embodiments, a fixation process involves formalin fixation followed by paraffin embedding.

In some embodiments, a tissue section has been permeabilized. Permeabilization facilitates access to cytoplasmic analytes such as RNA molecules. Thus, in some embodiments, a method comprises delivering permeabilization reagents (e.g., detergents such as Triton-X 100 or Tween-20) to a tissue section.

A tissue sample, in some embodiments, is sectioned. In some embodiments, a sectioned tissue sample is mounted on a substrate, such as a microscope slide, for example, a glass microscope slide, such as a polylysine-coated glass microscope slide. A tissue sample can be fixed before or after it is sectioned.

Aspects of the present disclosure relate to the application of spatial omics technology to clinically archived FFPE tissue sections. Clinically archived FFPE tissue sections contain an abundance of information that can be used to understand disease states or details about patient populations. A method of analyzing RNA molecules within frozen FFPE tissues also provides researchers with an option to collect tissue samples from a human or other organism and store the tissue samples for an indefinite period of time before proceeding with additional analyses.

### Microfluidic Devices

Microfluidic devices (e.g., chips) can be used, in some embodiments, to deliver barcoded polynucleotides to a tissue sample (e.g., tissue section) in a spatially defined manner. A system based on crossed microfluidic channels, such as those described herein, have several key parameters that largely determine the spatial resolution and mappable area of the device. These include (1) the number of microfluidic channels (n/eta); (2) the microchannel width (w/omega), measured in microns, i.e., the width of the open space in each microfluidic channel (tissue beneath these open spaces is imaged); and (3) microchannel pitch (A/delta), measured in microns, i.e., the width of the closed space between the end of one channel and the start of another channel (tissue beneath these closed spaces is not imaged). The microfluidic devices provided herein, in some embodiments, include multiple microchannels characterized by a width, depth, and pitch.

An exemplary detection scheme comprises two microfluidic devices. See, e.g., International Publication No. WO 2021/067246, Deterministic Barcoding for Spatial Omics Sequencing. For example, a first device flows reagents left to right and is drawn as a series of rows. A second device flows reagents from top to bottom and is drawn as a series of columns. The pixels of the detector comprise the overlap areas between the two sets of shapes-such a geometry endows the squares with edge length w microns. As an illustrative example, assume a detection scheme that utilizes microfluidic devices with η=50, ω=10 microns, and Δ=10 microns. In some embodiments, a detector features pixels that are squares with edge length 10 microns, and the distance between squares in the horizontal and vertical directions is equal to 20 microns. This means it can profile single cells that are approximately 10 microns or larger and resolve spatial features (e.g., characteristics of cell neighborhoods) that are 40 microns or larger. Microfluidic-based detectors display certain performance characteristics determined by the design and the design parameters. These include the following: (1) the ability to profile individual cells; (2) minimum length scale of spatial feature reproduction; and (3) the size of the mappable area.

These performance characteristics exert tension upon one another and therefore cannot be chosen independently. For example, it is possible to design a device with arbitrarily fine spatial resolution by decreasing w and A, even down to nanometer scale, as has been reported elsewhere. However, doing so would not result in a practical detector for examining tissue sections at single-cell resolution, as the mappable area of the device would be correspondingly small. On the other hand, drastically increasing the mappable area of the device by increasing w and A to very large values such as 1-2 mm (which has also been reported) would result in extremely coarse spatial resolution unsuitable for high spatial resolution imaging. Thus, there is a tradeoff between these design parameters that can be navigated to achieve a detector with both high spatial resolution and mappable area appropriately large for addressing the needs of the research community in investigating tissue samples with spatial features as small as cells but cell neighborhoods that can vary in biologically meaningful ways over distances of hundreds of microns.

One contributing factor to this tension is the fact that in a single-layer microfluidic device n, the number of channels, cannot be increased without limit. This is because each channel must be fed by inlets and lead to an outlet and must approach and recede from the region of interest without intersecting other channels on the same device. In some embodiments, it is possible to fit approximately 50 inlet and outlet ports while ensuring the device is still practical to fabricate and fill with reagents by hand.

In some embodiments, a first set of barcoded polynucleotides is delivered through a first microfluidic chip that comprises parallel microchannels positioned on a surface of the biological sample. In some embodiments, a first microfluidic chip comprises at least 5, at least 10, at least 20, at least 30, at least 40, or at least 50 parallel microchannels. In some embodiments, a first microfluidic chip comprises 5, 10, 20, 30, 40, or 50 parallel microchannels. In some embodiments, a first microfluidic chip comprises 5 to 100 parallel microchannels (e.g., 5-10, 5-25, 5-50, 5-75, 10-25, 10-50, 10-75, 10-100, 25-0, 25-27, 25-100, 50-75, or 50-100 parallel microchannels). In some embodiments, a second set of barcoded polynucleotides is delivered through a second microfluidic chip that comprises parallel microchannels that are positioned on the biological sample perpendicular to the direction of the microchannels of the first microfluidic chip. In some embodiments, a second microfluidic chip comprises at least 5, at least 10, at least 20, at least 30, at least 40, or at least 50 parallel microchannels. In some embodiments, a second microfluidic chip comprises 5, 10, 20, 30, 40, or 50 parallel microchannels. In some embodiments, a second microfluidic chip comprises 5 to 100 parallel microchannels (e.g., 5-10, 5-25, 5-50, 5-75, 10-25, 10-50, 10-75, 10-100, 25-0, 25-27, 25-100, 50-75, or 50-100 parallel microchannels). In some embodiments, a first set of barcoded polynucleotides comprises a sequence having 90% sequence identity to any one of SEQ ID NOs: 1-50. In some embodiments, a first set of barcoded polynucleotides comprises a sequence according to any one of SEQ ID NOs: 1-50. In some embodiments, a second set of barcoded polynucleotides comprises a sequence having 90% sequence identity to any one of SEQ ID NOs: 51-100. In some embodiments, a second set of barcoded polynucleotides comprises a sequence according to any one of SEQ ID NOs: 51-100.

In some embodiments, a microchannel has a width of at least 2 μm (e.g., at least 5 μm, at least 10 μm, at least 15 μm, at least 20 μm, at least 25 μm, at least 30 μm, at least 35 μm, at least 40 μm, or at least 50 μm). In some embodiments, a microchannel has a width of 2 μm, 5 μm, 10 μm, 15 μm, 20 μm, 25 μm, 30 μm, 35 μm, 40 μm, or 50 μm. In some embodiments, a microchannel has a width of 2 μm to 150 μm, 5 μm to 150 μm, or 2 μm to 150 μm (e.g., 10-125 μm, 10-100 μm, 25-150 μm, 25-125 μm, 25-100 μm, 50-150 μm, 50-125 μm, or 50-100 μm).

In some embodiments, a microchannel has a width of 2 μm to 150 μm near the inlet and outlet ports and a width of 2 μm to 50 μm near the region of interest. For example, a microchannel can have a width of 100 μm near the inlet and outlet ports and width of 50 μm near the region of interest. As another example, a microchannel can have a width of 100 μm near the inlet and outlet ports and width of 25 μm near the region of interest. As yet another example, a microchannel can have a width of 100 μm near the inlet and outlet ports and width of 10 μm near the region of interest. In some embodiments, a microchannel has a width of 2, 5, 10, 20, 25, 50, 60, 70, 80, 90, 100, 110, 120, 130, 130, 140, or 150 μm near the inlet and outlet ports. In some embodiments, a microchannel has a width of 2, 5, 10, 20, 30, 40, or 50 μm near the region of interest.

In some embodiments, a microchannel has a height of at least 2 μm (e.g., at least 2 μm, at least 5 μm, at least 10 μm, at least 15 μm, at least 20 μm, at least 25 μm, at least 30 μm, at least 35 μm, at least 40 μm, or at least 50 μm). In some embodiments, a microchannel has a height of 2 μm, 5 μm, 10 μm, 15 μm, 20 μm, 25 μm, 30 μm, 35 μm, 40 μm, or 50 μm). In some embodiments, a microchannel has a height of 2 μm to 150 μm (e.g., 10-125 μm, 10-100 μm, 25-150 μm, 25-125 μm, 25-100 μm, 50-150 μm, 50-125 μm, or 50-100 μm). These heights have been tested and shown to be enough to provide clearance above dust or tissue blockages, for example, and low enough to provide the required rigidity and to prevent deformation of the channel during clamping and flow.

In some embodiments, a microchannel has a width of 10 μm and a height of 12-15 μm. In other embodiments, a microchannel has a width of 25 μm and a height of 17-22 μm. In yet other embodiments, a microchannel has a width of 50 μm and a height of 20-100 μm.

Microchannel pitch is the distance between microchannels of a microfluidic device (e.g., chip). In some embodiments, the pitch of a microfluidic device is at least 10 μm (e.g., at least 15 μm, at least 20 μm, at least 25 μm, at least 30 μm, at least 35 μm, at least 40 μm, or at least 50 μm). In some embodiments, the pitch of a microfluidic device is at 10 μm, 15 μm, 20 μm, 25 μm, 30 μm, 35 μm, 40 μm, or 50 μm. In some embodiments, the pitch of a microfluidic device is at 10 μm to 150 μm (e.g., 10-125 μm, 10-100 μm, 25-150 μm, 25-125 μm, 25-100 μm, 50-150 μm, 50-125 μm, or 50-100 μm).

Many microfluidics platforms utilize positive pressure via syringe pumps, peristaltic pumps, and other types of positive pressure pumps whereby fluid is pumped from a reservoir into the device. Generally, a connection is made to interface the reservoir/pump assembly with the microfluidic device; often this takes the form of tubes terminating in pins that plug into inlet ports on the device. However, this type of system requires laborious and time-consuming fine-tuning of the assembly process associated with several drawbacks. For example, if the pins are inserted insufficiently deep into the inlet wells or the pin diameter is too small relative to the ports, then upon activation of the pumps, fluid pressure will eject the tube from the port. As another example, if the pins are inserted excessively deep into the wells, then upon activation of the pumps, fluid pressure will separate the microfluidic device from the glass substrate, resulting in leakage. While epoxying pins into ports and/or bonding the microfluidic device to the substrate via plasma bonding or thermal bonding might address the foregoing drawbacks, these strategies make it difficult to disassemble the system in a non-destructive way, resulting in component loss and are impractical when the substrate contains sensitive material, such as a tissue section, and/or antibodies.

In some embodiments, a negative pressure system is used, which utilizes a vacuum to pull liquid through the device from the back, rather than positive pressure to push it through the device from the front. This has several advantages, including, for example, (i) reducing the risk of leakage by pulling together the device and substrate and (ii) increasing efficiency and case of use—the vacuum can be applied to all outlet ports, unlike pins, which must be inserted individually into each inlet port. Using a negative pressure system saves several hours per run of fine-tuning and pin assembly.

Thus, in some embodiments provided herein, the barcoded polynucleotides are delivered to a region of interest through a microfluidic device (e.g., chip) using negative pressure (vacuum). In some embodiments, delivery of a first set of barcoded polynucleotides is delivered through a first microfluidic device using a negative pressure system. In some embodiments, delivery of a second set of barcoded polynucleotides is delivered through a second microfluidic device using a negative pressure system.

In some embodiments, a microfluidic device is clamped to a tissue section. Clamping the microfluidic device to the substrate in a localized manner, only above the region of interest, with a clamping force in the range of 5 to 50 newtons of force reduces leakage of reagents. In some embodiments, the clamping force is 5 to 50 newtons of force or 5 to 100 newtons of force (e.g., 5-75, 5-50, 5-25, 10-100, 10-75, 10-50, 10-25, 25-100, 25-75, 25-50, 50-100, 50-75, or 75-100 newtons of force, such as 5, 10, 15, 20, 25, 30, 35, 40, 45, 50, 55, 60, 65, 70, 75, 80, 85, 90, 95, or 100 newtons of force).

Microfluid chips, in some embodiments, are fabricated from polydimethylsiloxane (PDMS). Other substrates can be used.

### Methods

In some aspects, a method comprises producing spatially barcoded complementary deoxyribonucleic acids (cDNAs) from polyadenylated fragmented ribonucleic acids (RNAs) in a tissue section obtained from formalin-fixed paraffin-embedded (FFPE) tissue. This can include, for example, delivering a polyadenylate polymerase to the tissue section optionally with one or more polyadenylation reagents selected from polyadenylation specificity factors, cleavage stimulation factors, polyadenylate binding proteins, and cleavage factors. In some embodiments, producing spatially barcoded cDNAs comprises delivering reverse transcription reagents (e.g., reverse transcriptase) to the tissue section. In some embodiments, producing spatially barcoded cDNAs comprises delivering to the tissue section a first set of barcoded polynucleotides, a second set of barcoded polynucleotides, and ligation reagents to produce the spatially barcoded cDNAs.

The delivery of reagents to a tissue section, in preferred embodiments, is achieved using a microfluidic device, as described for example, in International Publication No. WO 2021/067246, Deterministic Barcoding for Spatial Omics Sequencing.

In some embodiments, a method comprises polyadenylating fragmented RNAs in a tissue section obtained from FFPE tissue to produce polyadenylated RNAs. This can include for example, delivering a polyadenylate polymerase and one or more polyadenylation reagents selected from polyadenylation specificity factors, cleavage stimulation factors, polyadenylate binding proteins, and cleavage factors. In some embodiments, polyadenylating fragmented RNAs includes producing cDNAs from the polyadenylated RNAs. This can include, for example, delivering reverse transcription reagents to the tissue section. In some embodiments, polyadenylating fragmented RNAs includes spatially barcoding the cDNAs to produce spatially barcoded cDNAs. This can include, for example, delivering to the tissue section a first set of barcoded polynucleotides, a second set of barcoded polynucleotides, and ligation reagents to produce the spatially barcoded cDNAs.

In some embodiments, a method comprises imaging a tissue section to produce a sample image. Imaging can be performed, for example, using an optical microscope or a fluorescence microscope. In some embodiments, a method comprises imaging a tissue section using an optical microscope. In some embodiments, a method comprises imaging a tissue section using a fluorescence microscope.

In some embodiments, a method comprises sequencing spatially barcoded cDNAs to produce sequencing reads. Non-limiting examples of sequencing techniques that can be used include Sanger Sequencing and Next-Generation Sequencing (NGS).

In some embodiments, the sequencing comprises template switching the cDNAs to add a second PCR handle end sequence at an end opposite from the first PCR handle end sequence, amplifying the cDNAs, producing sequencing constructs via tagmentation, and sequencing the sequencing constructs to produce the cDNA reads. In some embodiments, cDNAs originating from ribosomal RNA (rRNA) were selectively removed prior to sequencing. Methods for removing cDNAs originating from rRNA are known. In some embodiments, cDNAs are exposed to a blend of synthetic biotinylated oligonucleotides with homology to cDNA from both cytoplasmic and mitochondrial rRNAs to reduce or eliminate cDNAs originating from rRNA from within a sample of cDNAs. Template-switching (also known as template-switching polymerase chain reaction (TS-PCR)) is a method of reverse transcription and polymerase chain reaction (PCR) amplification that relies on a natural PCR primer sequence at the polyadenylation site, also known as the poly(A) tail, and adds a second primer through the activity of murine leukemia virus reverse transcriptase (see, e.g., Petalidis L. et al. Nucleic Acids Research. 2003; 31 (22): e142). Tagmentation refers to a modified transposition reaction, often used for library preparation, and involves a transposon cleaving and tagging double-stranded DNA with a universal overhang. Tagmentation methods are known.

In some embodiments, a method comprises mapping spatially barcoded cDNAs to points of origin within the tissue section. An exemplary method follows: Each spatially barcoded cDNA comprises a spatial barcode that is specific to a point within a tissue section. Sequencing of spatially barcoded cDNAs results in short computational sequences that represent sections of each spatially barcoded cDNA. Using computational analysis pipelines, for example those known in the art, short computational sequences that represent sections of spatially barcoded cDNAs are reconstructed to create full length computational sequences that represent each spatially barcoded cDNA. Each spatially barcoded cDNA comprises a UMI that represents a single cDNA molecule. Duplicate UMIs indicate that a single cDNA was duplicated by PCR error prior to sequencing. cDNA reads corresponding to duplicated UMIs are removed from the data set such that any given UMI occurs once in the data set. Following removal of duplicate UMIs, cDNA reads are aligned to a reference genome using computational methods known in the art. Sequence alignment results in spatially barcoded cDNAs mapped to genes within a reference genome. In some embodiments, a reference genome is derived from a mammalian genome. In some embodiments, a mammalian genome is a human genome or a rodent genome.

In some embodiments, mapping spatially barcoded cDNAs to points of origin within the tissue section comprises calculating gene expression levels based on sequencing reads. Following alignment of cDNAs to genes within a reference genome, cDNA counts for each gene within a reference genome can be calculated. “Counts” refer to the number of cDNA reads that correspond to a specific sequence within a reference genome. In some embodiments, a specific sequence within a reference genome corresponds to a gene. In some embodiments, a specific sequence within a reference genome corresponds to a splice variant. In some embodiments, a specific sequence within a reference genome corresponds to a product of adenosine-to-inosine (A-to-I) RNA editing. In some embodiments, a specific sequence within a reference genome corresponds to a miRNA. Computational methods for calculating and normalizing cDNA counts from cDNA reads are known. A first gene with more counts compared to a second gene is said to have a higher expression level than the second gene. In some embodiments, cDNA reads can be organized spatially into a spatial molecular expression map by computational methods.

In some embodiments, the methods comprise constructing a spatial molecular expression map of the biological sample by matching the spatially addressable barcoded conjugates to corresponding cDNA reads. In some embodiments, a spatial organization of cDNA reads is referred to a uniform manifold approximation and projection map (UMAP). Computational methods for spatially organizing cDNA reads are known and described in the Examples below. Spatial organization of cDNA reads results in a spatial molecular expression map that can be correlated to a sample image of a tissue section.

In some embodiments, methods comprise identifying the location of molecules of interest by correlating the spatial molecular expression map to a sample image. Each spatially barcoded cDNA comprises a spatially addressable barcode that corresponds to a point within a sample image of a tissue section. In some embodiments, a sample image of a tissue section is obtained prior to cDNA extraction. A sample image of a tissue section contains coordinates that match the locations of barcodes within a matrix of barcodes used to deliver barcoded polynucleotides to a tissue section. In some embodiments, a sample image of a tissue section is aligned to a matrix of barcodes. In some embodiments, a barcode within a matrix of barcodes is ligated to a cDNA within a tissue section. In some embodiments, a barcode within a matrix of barcodes is mappable to a sample image of a tissue section. Following computational analysis, for example using methods known in the art, a cDNA corresponding to a gene within a reference genome can be mapped to a specific point within a sample image of a tissue section by correlating a spatially barcoded cDNA to its point of origin within a matrix of barcodes which correlates to specific locations within a sample image of a tissue section. In some embodiments, a sample image of a tissue section comprises 20 μm pixels. In some embodiments, a sample image of a tissue section comprises 30 μm pixels. In some embodiments, a sample image of a tissue section comprises 40 μm pixels. In some embodiments, a sample image of a tissue section comprises 50 μm pixels. A spatially barcoded cDNA corresponding to a gene is spatially addressable to a pixel within a sample image of a tissue section. In some embodiments, each pixel within a sample image is mapped to at least 1,000, at least 2,000, at least 3,000, at least 4,000, at least 5,000, at least 6,000, at least 7,000, or at least 8,000 genes. Once cDNAs are correlated to pixels within a sample image, gene expression data from a spatial molecular expression map can be correlated to locations within the sample image to determine expression levels of a molecule of interest in a location of interest within the sample image. Examples of these methods steps are described in the Examples below.

Also provided herein are compositions produced using one or more methods of the disclosure. In some embodiments, such compositions comprise a processed tissue section (e.g., an FFPE tissue section) comprising spatially barcoded cDNAs and polyadenylated fragmented RNAs.

Additional embodiments of the disclosure are described in the numbered paragraphs below:

Paragraph 1. A method, comprising: (a) producing spatially barcoded complementary deoxyribonucleic acids (cDNAs) from polyadenylated fragmented ribonucleic acids (RNAs) in a tissue section obtained from formalin-fixed paraffin-embedded (FFPE) tissue; and (b) mapping the spatially barcoded cDNAs to points of origin within the tissue section.

Paragraph 2. The method of Paragraph 1, wherein (a) comprises: (i) delivering a polyadenylate polymerase to the tissue section, and optionally delivering to the tissue section a polyadenylation reagent selected from polyadenylation specificity factors, cleavage stimulation factors, polyadenylate binding proteins, and cleavage factors; (ii) delivering reverse transcription reagents to the tissue section; and (iii) delivering to the tissue section a first set of barcoded polynucleotides, a second set of barcoded polynucleotides, and ligation reagents to produce spatially barcoded cDNAs.

Paragraph 3. A method, comprising: (a) polyadenylating fragmented RNAs in a tissue section obtained from formalin-fixed paraffin-embedded (FFPE) tissue to produce polyadenylated RNAs; (b) producing cDNAs from the polyadenylated RNAs; (c) spatially barcoding the cDNAs to produce spatially barcoded cDNAs; and (d) mapping the spatially barcoded cDNAs to points of origin within the tissue section.

Paragraph 4. The method of any one of the preceding Paragraphs, wherein the fragmented RNAs are selected from the group consisting of mRNAs, ribosomal RNAs, transfer RNAs, microRNAs, long noncoding RNAs, small noncoding RNAs, small nuclear RNA, and piwi RNA.

Paragraph 5. The method of Paragraph 3 or 4, wherein (a) comprises delivering a polyadenylate polymerase to the tissue section, and optionally delivering to the tissue section a polyadenylation reagent selected from polyadenylation specificity factors, cleavage stimulation factors, polyadenylate binding proteins, and cleavage factors.

Paragraph 6. The method of any one of Paragraphs 3-5, wherein (b) comprises delivering reverse transcription reagents to the tissue section.

Paragraph 7. The method of any one of Paragraphs 3-6, wherein (c) comprises delivering to the tissue section a first set of barcoded polynucleotides, a second set of barcoded polynucleotides, and ligation reagents to produce the spatially barcoded cDNAs.

Paragraph 8. A method, comprising: (a) delivering a polyadenylate polymerase to a tissue section obtained from formalin-fixed paraffin-embedded (FFPE) tissue to produce polyadenylated ribonucleic acids (RNAs); (b) delivering reverse transcription reagents to the tissue section to produce cDNAs; (c) delivering to the tissue section a first set of barcoded polynucleotides, a second set of barcoded polynucleotides, and ligation reagents to produce spatially barcoded cDNAs; (e) imaging the tissue section to produce a sample image; (f) sequencing the spatially barcoded cDNAs to produce sequencing reads; and (g) mapping the spatially barcoded cDNAs to points of origin within the tissue section.

Paragraph 9. The method of Paragraph 8, wherein (a) further comprises delivering to the tissue section a polyadenylation reagent selected from polyadenylation specificity factors, cleavage stimulation factors, polyadenylate binding proteins, and cleavage factors.

Paragraph 10. The method of any one of Paragraphs 2, 6, and 7, wherein the first set of barcoded polynucleotides and the second set of barcoded polynucleotides are delivered using a microfluidic device, optionally made from polydimethylsiloxane (PDMS).

Paragraph 11. The method of Paragraph 10, wherein the microfluidic device comprises a first component for delivery of the first set of barcoded polynucleotides and a second component for delivery of the second set of barcoded polynucleotides, each of the components comprising parallel variable width microchannels.

Paragraph 12. The method of any one of the preceding Paragraphs, wherein the tissue section has been permeabilized.

Paragraph 13. The method of Paragraph 12, wherein the tissue section was frozen prior to being permeabilized.

Paragraph 14. The method of any one of the preceding Paragraphs, wherein the tissue section is mounted on a microscope slide.

Paragraph 15. The method of any one of the preceding Paragraphs, wherein the FFPE tissue is mammalian tissue, optionally a human tissue.

Paragraph 16. The method of any one of the preceding Paragraphs, wherein the FFPE tissue is bacterial tissue.

Paragraph 17. The method of Paragraph 16, wherein each of the first component and the second component comprises 5-1000 variable width microchannels, each of the microchannels having (i) an inlet port and an outlet port, (ii) a width of 2-150 μm, at the inlet port and the outlet port, and (iii) a width of 2-50 μm at the tissue section.

Paragraph 18. The method of Paragraph 16 or 17, wherein the first component and the second component are oriented at an angle of greater than 10 degrees relative to each other during delivery of the first set of barcoded polynucleotides and the second set of barcoded polynucleotides.

Paragraph 19. The method of Paragraph 18, wherein the first component and the second component are oriented perpendicular relative to each other during delivery of the first set of barcoded polynucleotides and the second set of barcoded polynucleotides.

Paragraph 20. The method of any one of the preceding Paragraphs, wherein the imaging is with an optical microscope or a fluorescence microscope.

The follow numbered embodiments are also provided:

1. A method, comprising:
(a) producing spatially barcoded complementary deoxyribonucleic acids (cDNAs) from poly(N)-tailed fragmented ribonucleic acids (RNAs) in a tissue section obtained from formalin-fixed paraffin-embedded (FFPE) tissue; and
(b) mapping the spatially barcoded cDNAs to points of origin within the tissue section.

2. The method of embodiment 1, wherein (a) comprises:
(i) delivering a poly(N) polymerase to the tissue section;
(ii) delivering reverse transcription reagents to the tissue section; and
(iii) delivering to the tissue section a first set of barcoded polynucleotides, a second set of barcoded polynucleotides, and ligation reagents to produce spatially barcoded cDNAs.

3. A method, comprising:
(a) poly(N)-tailed fragmented RNAs in a tissue section obtained from formalin-fixed paraffin-embedded (FFPE) tissue to produce polyadenylated RNAs;
(b) producing cDNAs from the poly(N)-tailed RNAs;
(c) spatially barcoding the cDNAs to produce spatially barcoded cDNAs; and
(d) mapping the spatially barcoded cDNAs to points of origin within the tissue section.

4. The method of any one of the preceding embodiments, wherein the fragmented RNAs are selected from the group consisting of mRNAs, ribosomal RNAs, transfer RNAs, microRNAs, long noncoding RNAs, small noncoding RNAs, small nuclear RNA, and piwi RNA.

5. The method of embodiment 3 or 4, wherein (a) comprises delivering a poly(N) polymerase to the tissue section.

6. The method of any one of embodiments 3-5, wherein (b) comprises delivering reverse transcription reagents to the tissue section.

7. The method of any one of embodiments 3-6, wherein (c) comprises delivering to the tissue section a first set of barcoded polynucleotides, a second set of barcoded polynucleotides, and ligation reagents to produce the spatially barcoded cDNAs.

8. A method, comprising:
(a) delivering a poly(N) polymerase to a tissue section obtained from formalin-fixed paraffin-embedded (FFPE) tissue to produce poly(N)-tailed ribonucleic acids (RNAs);
(b) delivering reverse transcription reagents to the tissue section to produce CDNAs;
(c) delivering to the tissue section a first set of barcoded polynucleotides, a second set of barcoded polynucleotides, and ligation reagents to produce spatially barcoded CDNAs;
(e) imaging the tissue section to produce a sample image;
(f) sequencing the spatially barcoded cDNAs to produce sequencing reads; and
(g) mapping the spatially barcoded cDNAs to points of origin within the tissue section.

9. The method of embodiment 8, wherein (a) further comprises delivering to the tissue section a reagent selected from specificity factors, cleavage stimulation factors, binding proteins, and cleavage factors.

10. The method of embodiment 8 or 9, wherein the imaging is with an optical microscope or a fluorescence microscope.

11. The method of any one of embodiments 2, 6, and 7, wherein the first set of barcoded polynucleotides and the second set of barcoded polynucleotides are delivered using a microfluidic device, optionally made from polydimethylsiloxane (PDMS).

12. The method of embodiment 11, wherein the microfluidic device comprises a first component for delivery of the first set of barcoded polynucleotides and a second component for delivery of the second set of barcoded polynucleotides, each of the components comprising parallel variable width microchannels.

13. The method of any one of the preceding embodiments, wherein the tissue section has been permeabilized.

14. The method of embodiment 13, wherein the tissue section was frozen prior to being permeabilized.

15. The method of any one of the preceding embodiments, wherein the tissue section is mounted on a microscope slide.

16. The method of any one of the preceding embodiments, wherein the FFPE tissue is mammalian tissue, optionally human tissue.

17. The method of any one of the preceding embodiments, wherein the FFPE tissue is bacterial tissue.

18. The method of embodiment 17, wherein each of the first component and the second component comprises 5-1000 variable width microchannels, each of the microchannels having (i) an inlet port and an outlet port, (ii) a width of 2-150 μm, at the inlet port and the outlet port, and (iii) a width of 2-50 μm at the tissue section.

19. The method of embodiment 17 or 18, wherein the first component and the second component are oriented at an angle of greater than 10 degrees relative to each other during delivery of the first set of barcoded polynucleotides and the second set of barcoded polynucleotides.

20. The method of embodiment 19, wherein the first component and the second component are oriented perpendicular relative to each other during delivery of the first set of barcoded polynucleotides and the second set of barcoded polynucleotides.

21 The method of any one of the preceding embodiments, wherein the mapping comprises:
(i) calculating gene expression levels based on sequencing reads;
(ii) constructing a spatial molecular expression map by correlating gene expression levels to spatial sequences within the sequencing reads; and
(iii) correlating the spatial molecular expression map to the sample image.

22. The method of embodiment 21, wherein calculating gene expression levels comprises aligning sequencing reads to a reference genome.

23 The method of embodiment 22, wherein the reference genome is derived from a mammalian genome.

24. The method of embodiment 23, wherein the mammalian genome is a human genome or a rodent genome.

25. The method of any one of embodiments 21-24, wherein constructing the spatial molecular expression map comprises generating a uniform manifold approximation and projection map (UMAP).

26 The method of any one of embodiments 21-25, wherein step (iii) further comprises correlating spatial sequences within the sequencing reads to locations within the sample image.

27. The method of any one of embodiments 2-26, wherein the first set of barcoded polynucleotides comprises a sequence having 90% sequence identity to any one of SEQ ID NOs: 1-50.

28 The method of embodiment 27, wherein the first set of barcoded polynucleotides comprises a sequence according to any one of SEQ ID NOs: 1-50.

29 The method of any one of embodiments 2-28, wherein the second set of barcoded polynucleotides comprises a sequence having 90% sequence identity to any one of SEQ ID NOS: 51-100.

30 The method of embodiment 29, wherein the second set of barcoded polynucleotides comprises a sequence according to any one of SEQ ID NOs: 51-100.

### EXAMPLES

Further details can be found in the manuscript entitled, Spatially exploring RNA biology in archival formalin-fixed paraffin-embedded tissues; Bai et al. (Nov. 14, 2024), Vol. 187, pp. 6760-6779, including FIGS. 1-7, Supplemental Figures S1-S8, and any Supplemental information referenced therein, the entire contents of which are incorporated herein by reference. The capacity to spatially explore RNA biology in processed tissues, such as formalin-fixed paraffin-embedded (FFPE) tissues, holds transformative potential for pathology research. Here, Patho-DBIT was presented by combining in situ polyadenylation and deterministic barcoding for spatial whole transcriptome sequencing, tailored for probing the diverse landscape of RNA species in clinically archived FFPE samples, for example. Patho-DBiT permits spatial co-profiling of gene expression and RNA processing, unveiling region-specific isoforms in the mouse brain. High-sensitivity transcriptomics is constructed from clinical tissues stored for five years. Furthermore, genome-scale single nucleotide RNA variants are captured to distinguish malignant from non-malignant cells in human lymphomas. Patho-DBIT also maps microRNA-mRNA regulatory networks and RNA splicing dynamics, decoding their roles in spatial tumorigenesis and developmental trajectory. High resolution Patho-DBiT at the cellular level reveals a spatial neighborhood and traces the spatiotemporal kinetics driving tumor progression. Patho-DBIT stands poised as a valuable platform to unravel rich RNA biology in FFPE tissues to aid pathology diagnosis.

### Example 1. Patho-DBiT Design, Performance, and Spatial Mapping of Mouse Embryo

The Patho-DBiT method was initiated with tissue section deparaffinization and heat-induced crosslink retrieval, adhering to a standardized protocol (FIGS. 1A-1B). After tissue permeabilization, enzymatic in situ polyadenylation enabled detection of the full spectrum of RNAs, followed by cDNA strand synthesis by reverse transcription. Spatial barcoding was then achieved using a microfluidic device with two PDMS chips featuring 50 parallel microchannels. These channels sequentially delivered horizontal (A1-A50) and perpendicular (B1-B50) barcodes, creating a unique 2D barcode combination array. Post-imaging, the tissue underwent digestion to extract barcoded cDNA to perform the downstream procedures including template switch and PCR amplification. Polyadenylation added poly(A) tails to all RNAs, including the predominant ribosomal RNA (rRNA) constituting 80-90% of cellular RNA, yet it provided limited information on the target transcriptome. To circumvent the loss of rare or low-abundance transcripts, cDNA fragments originating from rRNA were selectively removed from amplicons. This was achieved by employing a blend of synthetic biotinylated oligos with homology to cDNA from both cytoplasmic and mitochondrial rRNAs, resulting in the substantial elimination of these fragments prior to library sequencing (figure not shown).

To benchmark the technology, Patho-DBIT was applied on an embryonic day 13 (E13) mouse embryo FFPE section using the microfluidic device with a resolution of 50 μm. Unsupervised clustering revealed 20 transcriptomic clusters, and the spatial Uniform Manifold Approximation and Projection (UMAP) closely aligned with the histology of an adjacent section stained with hematoxylin and cosin (H&E) (FIG. 1C). Cell type-specific marker genes of each individual cluster were identified, and their expression was uniquely represented in each cluster, which could be clearly separated with other clusters. Cell type-specific marker genes were identified, uniquely characterizing their expression within each individual cluster for clear differentiation from other groups (figure not shown). Notably, the distribution of these clusters exhibited conspicuous and distinctive spatial patterns, underscoring the high sensitivity and accuracy of this assay (figure not shown). Patho-DBiT detected an average of 5,480 genes and 15,381 unique molecular identifiers (UMIs) per 50 μm pixel, with the genome-wide pan-mRNA and UMI maps displaying a strong alignment with tissue morphology and density (FIG. 1D). Reproducibility among replicates was notably high, as reflected by a Pearson correlation coefficient of 0.999. (FIG. 1E). To assess the read coverage across gene bodies in the technology, replicated datasets were generated on adjacent E13 sections using normal DBiT-seq without polyadenylation. While still exhibiting a 3′ bias, Patho-DBiT showcased an approximate twofold increase in coverage across the entire gene body, and the percentage of reads mapped to the 5′ untranslated region (UTR) more than doubled (FIG. 1F). This observation substantiates the capture of more RNA molecules throughout the maturation cycle. Additionally, with a higher number of unique genes detected, Patho-DBiT maintained a comparably low level of reads mapped to rRNA (FIG. 1G), reaffirming the efficacy of removing this undesired category.

For a more thorough assessment of data quality and to discern the cell identities within each spatial cluster, the datasets were integrated with scRNA-seq reference data from E13.5 mouse organogenesis. This integration yielded a cohesive pattern, aligning the spatial pixels with the scRNA-seq dataset in a well-conformed manner (FIG. 1H). For example, cluster 1 in the spinal cord part and clusters 2, 3, and 4 located in the facial area were accurately assigned to osteoblasts, highlighting their involvement in the bone-forming process. Cluster 5 cells seamlessly integrated with myocytes, mirroring their role in skeletal muscle development. Clusters 6, 12, and 18 associated with the central nervous system (CNS) accurately mapped to neurons or oligodendrocytes. Cells in cluster 7, located in the liver region, uniquely integrated with the definitive erythroid lineage, marking their association with red blood cell development initiated in this organ. Furthermore, within the liver region, cluster 9 cells were distinctly assigned to hepatocytes, contributing to the liver's structural integrity and function. Additionally, cells in clusters 11, 14, and 17, linked to connective tissues or cartilage formation, were accurately identified as stromal cells. Cells located in the heart region within cluster 15 were precisely inferred as cardiac muscle lineages. These findings reinforced Patho-DBiT's high accuracy in cell type detection and spatial localization within the elaborate landscape of the developing mouse embryo.

To demonstrate its capability in detecting small RNA, the microRNA profile was scrutinized in the E13 mouse embryo sample. MicroRNAs, small single-stranded non-coding RNA molecules comprising 21 to 23 nucleotides, were analyzed as a representative case. In total, Patho-DBiT detected 1063 microRNAs, peaking at 22 nucleotides in the count of mapped reads within the dataset. The average counts of UMIs and microRNAs in each pixel were 60 and 30, respectively (figure not shown). The spatial distribution of microRNA UMI and pan-microRNA count showed a notable correlation with that of mRNA, implying coherent expression patterns between large and small RNAs within a spatial context (figure not shown). To validate the mapping accuracy, miR-122 was assessed as one of the earliest examples of a tissue-specific microRNA, constituting 70% of the total microRNA pool in the liver. With reads precisely aligned to its reference genome location, miR-122 exhibited a markedly higher expression proportion in the two liver region clusters, uniquely enriching its spatial distribution within this specific area (figure not shown). The expression landscape of the let-7 family of microRNAs was also reviewed as it plays pivotal roles in mouse embryonic development. Patho-DBiT detected 11 out of 14 members of this family, with heterogenous expressions in different spatial clusters (figure not shown).

The Patho-DBIT was applied across diverse tissue types and spatial resolutions, yielding remarkable results. At a 50 μm resolution, the approach exhibited superior performance compared to the probe-based 10× Genomics Visium for FFPE at a 55 μm feature size, even surpassing its fresh frozen counterpart reliant on conventional 3′-targeted barcoding of polyadenylated RNAs. Over 4,000 genes per pixel were consistently identified in lymphoma sections at this resolution and more than 3,000 genes in samples from mouse lymph nodes at the 20 μm resolution. Notably, employing the microfluidic device with 10 μm channels, 2,292 genes and 6,021 UMIs were identified from a lymphoma section at this near-cellular level. This accomplishment exceeded the capture efficiency of several state-of-the-art technologies employed on fresh frozen samples at the specified resolution, such as Sterco-seq (4.1-fold), Seq-Scope (>6-fold), and Slide-scqV224 (>10-fold).

### Example 2. Spatial Co-Profiling of Gene Expression, Alternative Splicing and A-to-I RNA Editing in the Mouse Brain

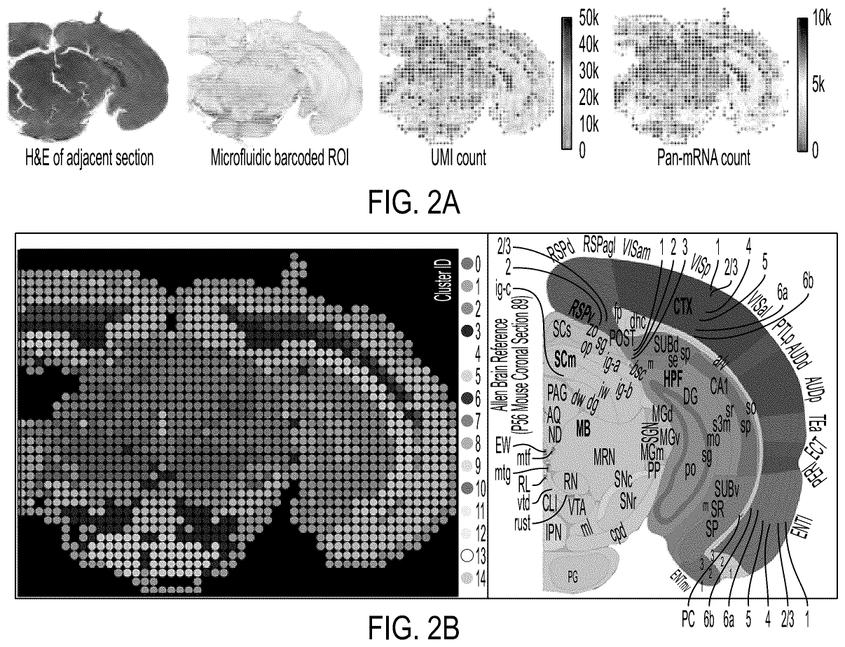

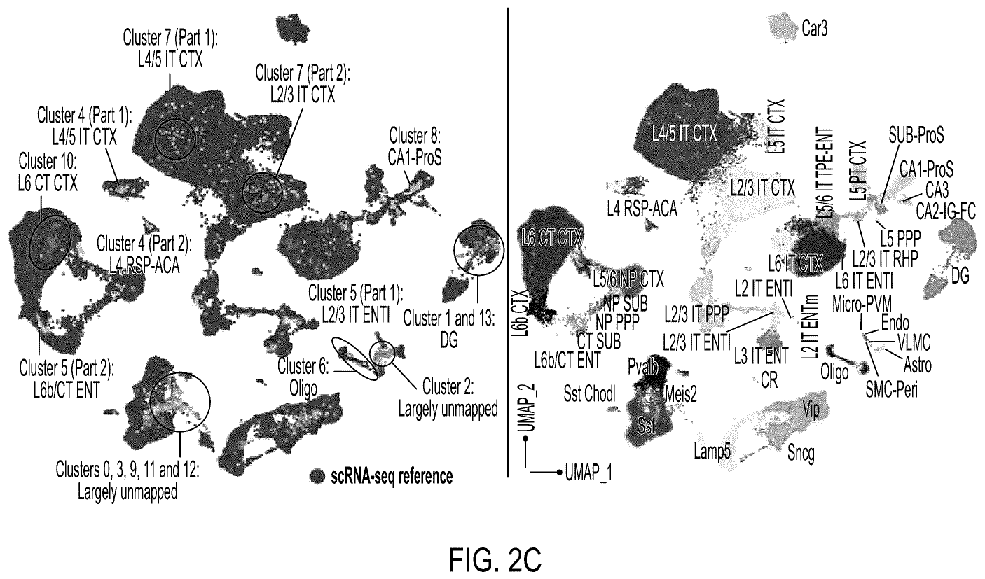

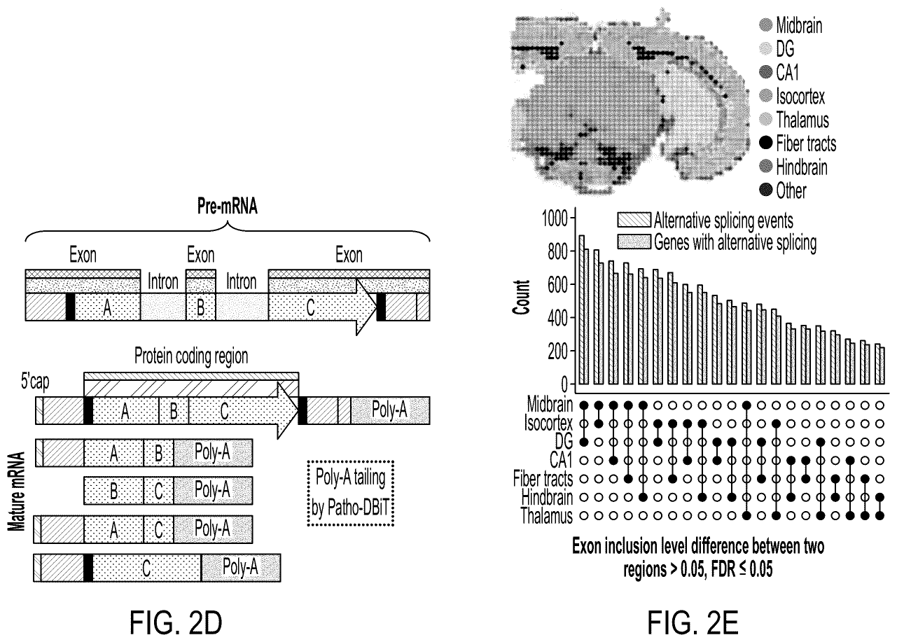

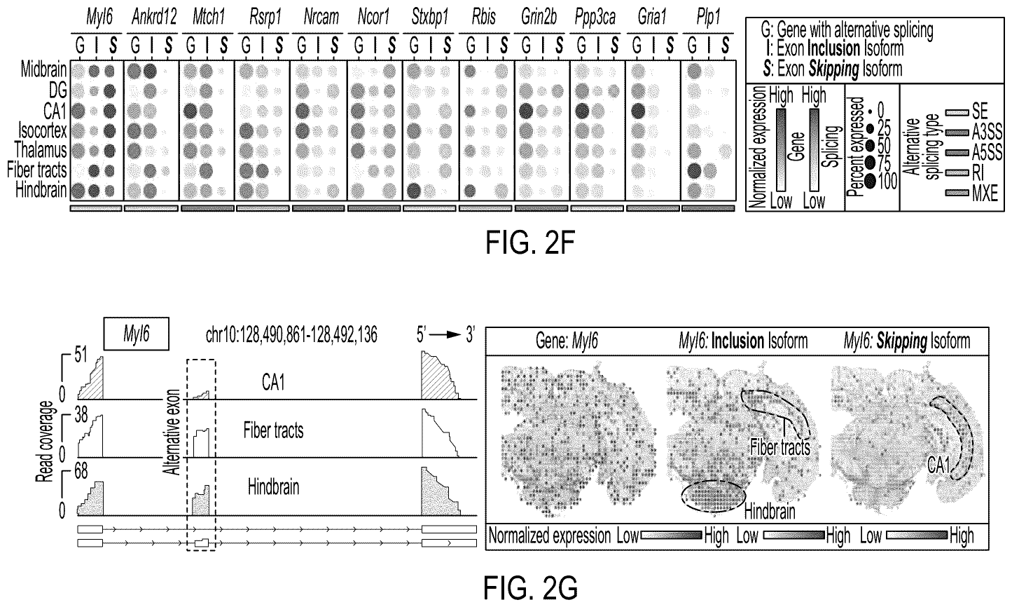

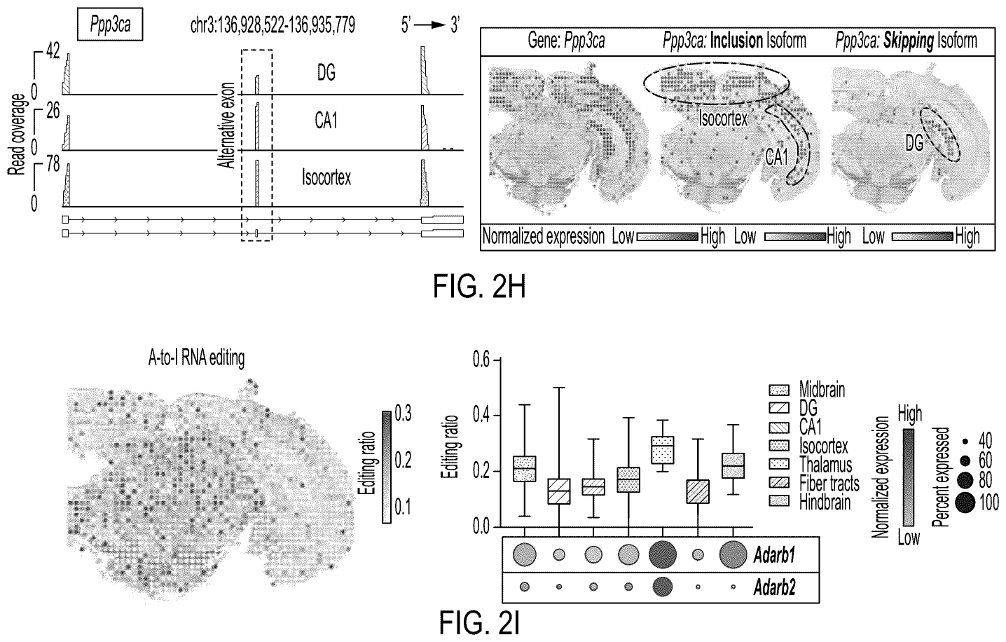

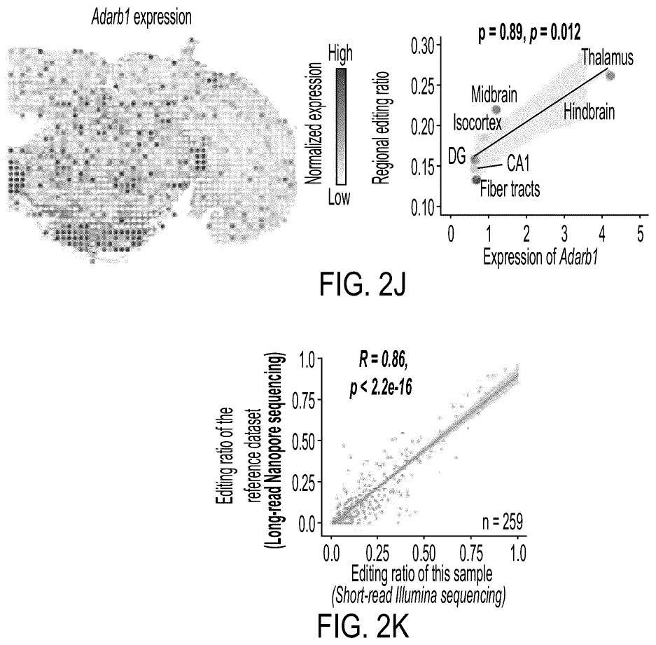

To further demonstrate the robustness of Patho-DBIT and evaluate its capability to simultaneously map gene expression and post-transcriptional modifications such as alternative splicing and RNA editing, a FFPE mouse coronal brain section was profiled. An average of 6,786 genes and 31,063 UMIs were detected per 50 μm pixel, exhibiting a spatial distribution strongly correlated with the tissue histology outlined by the H&E staining of an adjacent section (FIG. 2A). Clustering analysis, utilizing gene expression matrix, unveiled 15 anatomical clusters characterized by unique gene markers expressed in each subpopulation, and the spatial arrangements broadly aligned with the region annotations on a similar section from the Allen Mouse Brain Atlas (FIG. 2B). Remarkably, the isocortex area was precisely deconstructed into three layers, assigning cluster 7 to layer 1-2, cluster 4 to layer 4-5, and cluster 10 to layer 6a-b. The spatial expression pattern of the primary defining gene in each cluster closely mirrored the in situ hybridization (ISH) results for the same genes (figure not shown), underscoring Patho-DBiT's capacity to faithfully refine tissue structures.

Integration and co-embedding the Patho-DBIT data with the scRNA-seq atlas from cells in the mouse cortex and hippocampus validated the identity of these clusters (FIG. 2C). Specifically, cells in clusters 1 and 13 integrated with the dentate gyrus (DG) type, corresponding to DG-molecular layer and DG-polymorph layer, respectively. Clusters 4, 7, and 10 consistently mapped to different layers of the isocortex, as previously described. Cluster 5 cells were also situated in the isocortex region, exhibiting a notably accurate classification as cither L2/3 or L6b entorhinal area (ENT) cells. Cluster 6 cells were uniquely identified as oligodendrocytes (oligo), correlating with their distribution in the fiber tract areas. Similarly, exclusive identification was noted for cluster 8 cells, revealing their identity as hippocampal CA1 prosubiculum (CA1-Pros) cells and spatial representation. Cells in clusters 0, 3, 9, 11, and 12, located in the midbrain or hindbrain areas, remained largely unmapped due to the absence of cells from these regions in the reference scRNA-seq dataset. This provides further evidence supporting the high sensitivity and specificity of Patho-DBIT.

Alternatively spliced transcripts play a crucial role in neurogenesis and brain development, contributing to the intricate architecture of the mammalian CNS by regulating a diverse range of neuronal functions. However, identifying splicing events from short-read RNA-seq data remains elusive due to the requirement of adequate read coverage for reliable capture of the splicing junction-spanning region. Through the addition of a poly(A) tail during the RNA transcription process (FIG. 2D), Patho-DBiT exhibited remarkably broader coverage across the gene body than another poly(T) capture-based approach, 10× Genomics Visium, on a fresh frozen mouse brain section (figure not shown). A total of 3,879 distinct splicing events encoded by 2,368 genes were detected, covering all the major event types including skipped exon (SE), retained intron (RI), alternative 3′ splice site (A3SS), alternative 5′ splice site (A5SS), and mutually exclusive exons (MXE) (figure not shown). On average, each spatial spot yielded 105 splicing events corresponding to 85 parental genes, with a mean detection level of 43 UMIs per event (figure not shown). This performance on FFPE sections was 2.2-fold higher compared to the Visium counterpart on frozen sections. In contrast, the probe-based Visium FFPE solution showed limited detection of splicing events, likely attributed to its discrete capture despite a relatively broad coverage (figure not shown).

Splicing patterns with significant changes across brain regions were explored to unravel their spatial organizational differences. Utilizing criteria set by rMATS (see STAR Methods), an exon inclusion level difference >0.05 between two regions with a false discovery rate (FDR) of ≤0.05 were deemed as significant. A minimum of 220 distinct events was identified between two brain regions (specifically observed between hindbrain and thalamus), with the splicing pattern of midbrain and DG exhibiting the most pronounced differences compared to other pairs, potentially linked to their highly specialized functions within the complex brain system (FIG. 2E). SE was the most abundant event type showing significant variations across regions (figure not shown). The top-ranked genes that are involved in brain functions and have pronounced regional isoform switching were reviewed (FIG. 2F). For example, Myl6, a gene widely involved in neuronal migration and synaptic remodeling with a uniform distribution across the entire section, exhibits an enriched inclusion isoform in the fiber tracts and hindbrain, in contrast to the skipping isoform enriched in CA1 (FIG. 2G). Ppp3ca has been identified as a leading modulator of genetic risk in Alzheimer's disease. The data unveiled distinct isoform usage patterns, with the inclusion isoform prevailing in the isocortex and CA1, while the skipping isoform was exclusively expressed in the DG (FIG. 2H). Patho-DBIT also identified notable examples of spatial isoform distribution, including Nrcam and Stxbp1, functional genes regulating neural development and disorders and neurotransmitter release, respectively (figure not shown).

Transcriptomic diversity blossomed through adenosine-to-inosine (A-to-I) RNA editing, an essential process vital for proper neuronal function. With superior read coverage, Patho-DBiT spatially mapped A-to-I editing in situ on FFPE brain sections, unveiled a distinctive editing ratio landscape across different regions (FIG. 2I). Conspicuous variations emerged, with thalamus exhibiting a notably elevated editing ratio (mean 27.9%), while fiber tracts displayed a lower ratio (mean 12.7%). This pattern closely corresponds to the expression levels and frequencies of genes (Adarb1, Adarb2) dedicated to encoding A-to-I editing enzymes, known as adenosine deaminases (ADARs). Particularly, the spatial distribution of Adarb1 closely mirrored the editing ratio, yielding a robust Spearman correlation coefficient of 0.89 (FIG. 2J). To validate the accuracy of these findings, the editing ratio was cross-referenced with a published dataset generated from fresh frozen coronal brain sections using long-read nanopore sequencing. A substantial Pearson correlation score of 0.86 was observed across 259 commonly detected editing sites showing at least 10 UMIs in both datasets (FIG. 2K and data not shown). Therefore, Patho-DBiT provided a comprehensive spatial delineation of the transcriptome, alternative splicing, and A-to-I editing in the FFPE mouse brain section.

### Example 3. Patho-DBiT Recapitulates Underlying Lymphomagenesis Biology in Clinical Archival AITL Sample

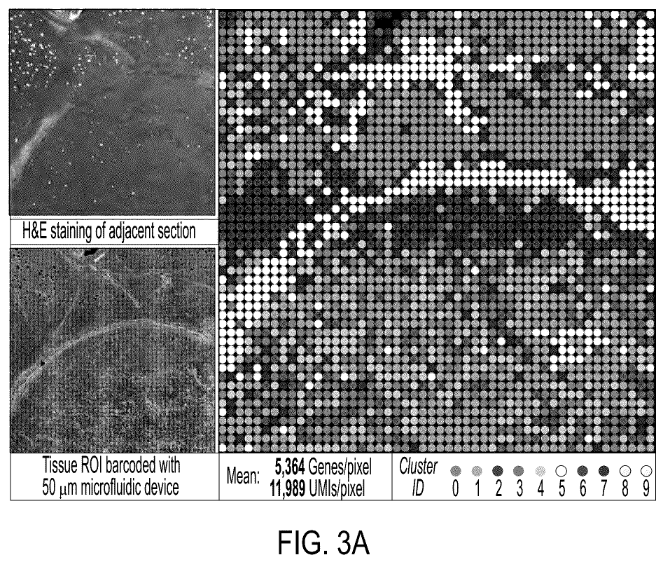

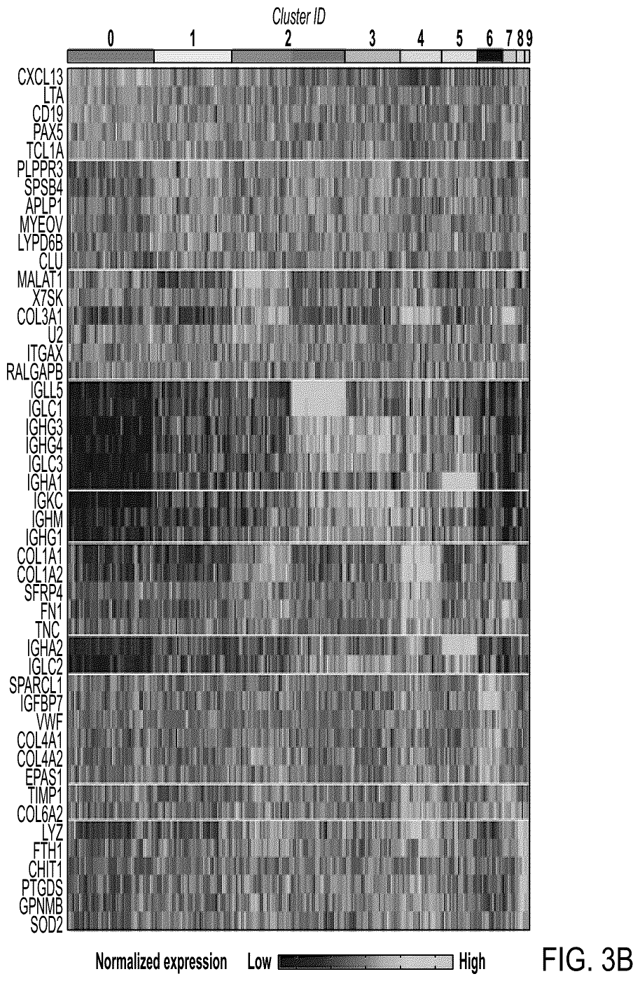

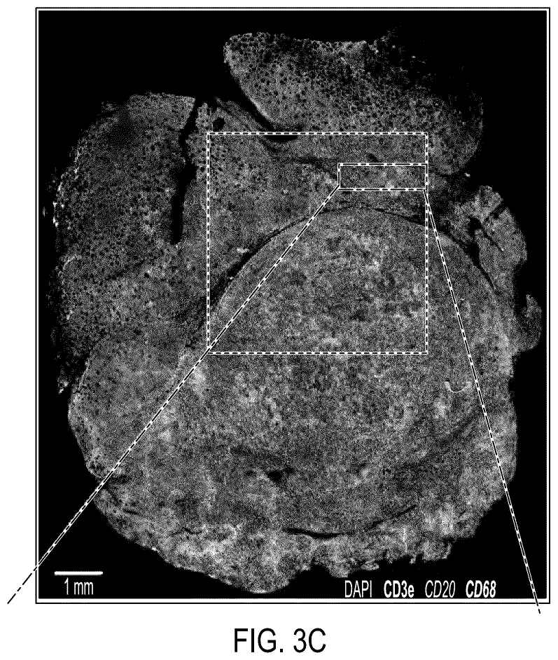

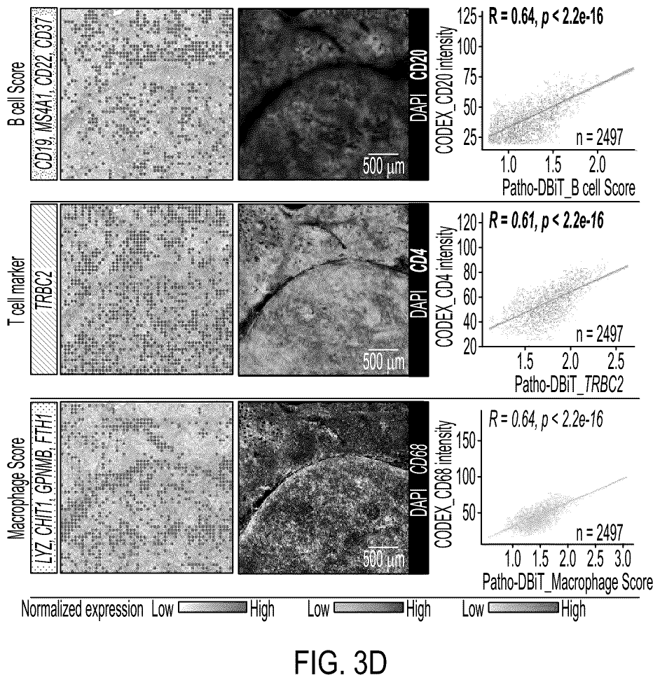

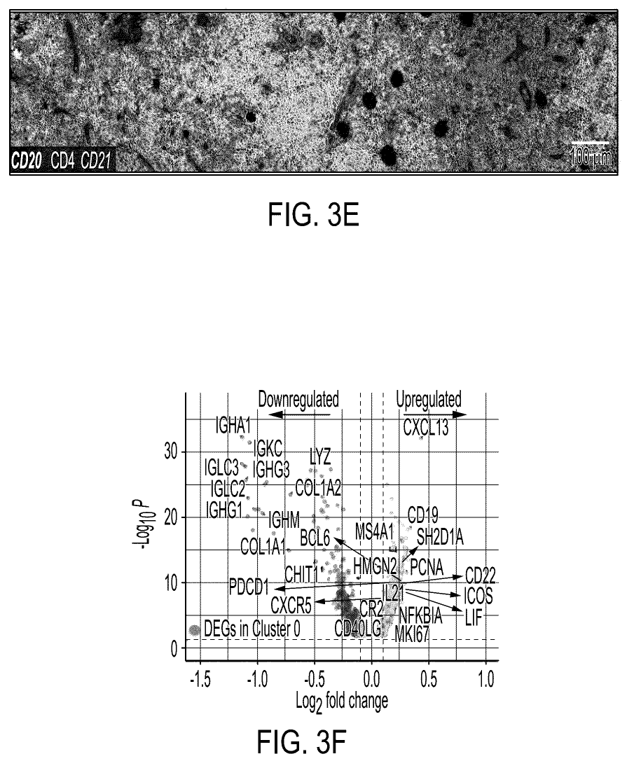

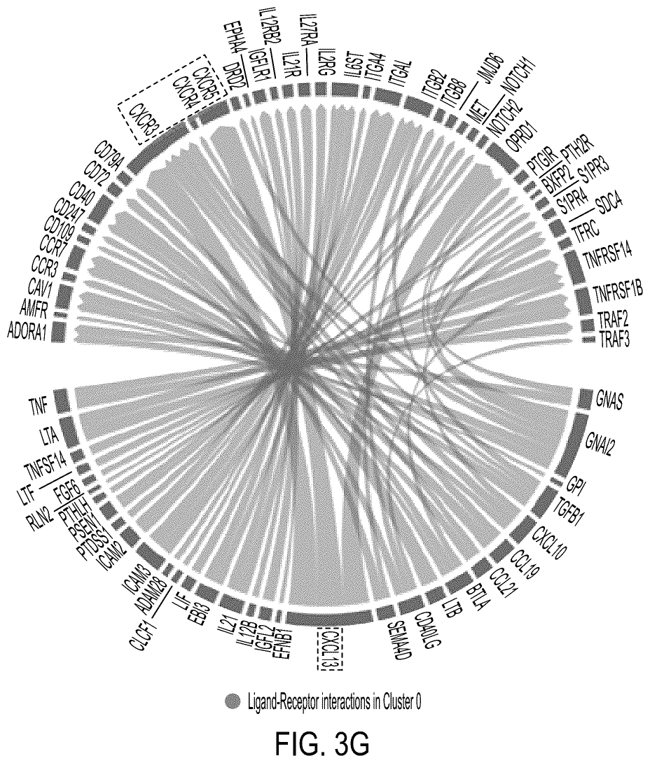

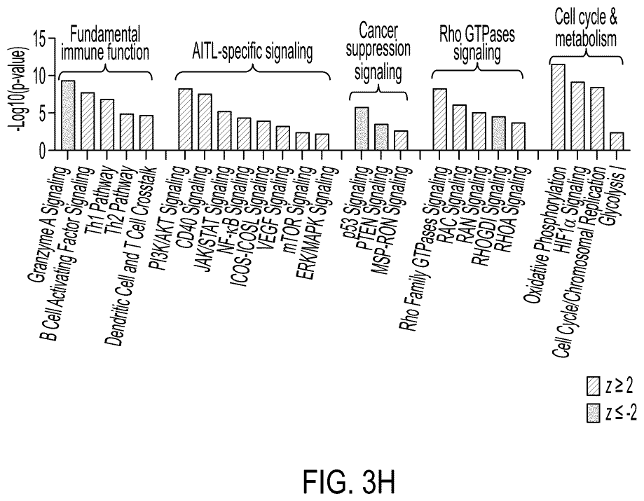

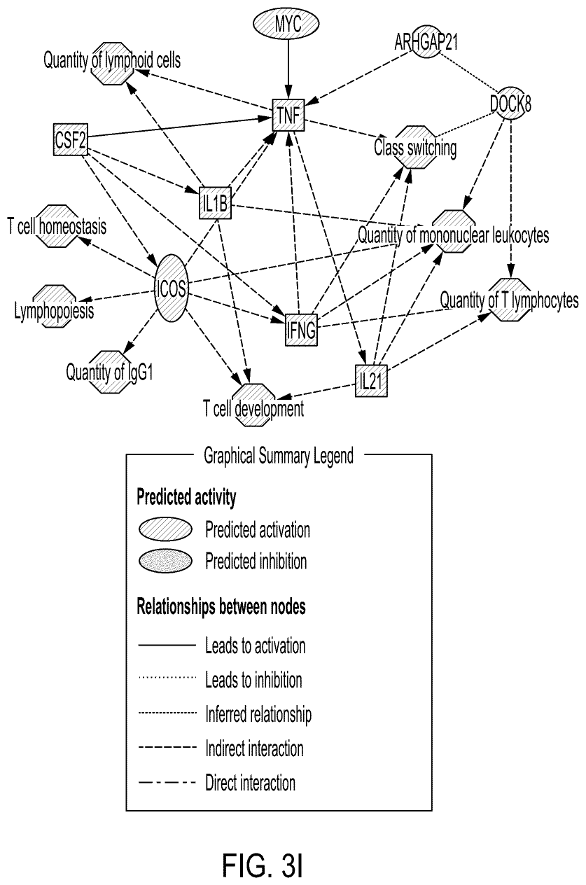

Next, the spatial profiling was extended to clinically archived FFPE tissues. A 50 μm resolution Patho-DBIT device was employed to barcode a section obtained from the subcutaneous nodule of a 73-year-old patient diagnosed with angioimmunoblastic T-cell lymphoma (AITL) affecting multiple lymph nodes and subcutaneous sites (figure not shown). This block had been stored at room temperature for over five years prior to our assay. Unsupervised clustering of the gene-by-pixel matrix revealed 10 spatially organized clusters aligned with histological structures (FIG. 3A). The UMAP, with an average of 5,364 genes and 11,989 UMIs per pixel, delineated distinct cell types defined by canonical markers (3B). Notably, cluster 5 exhibited a high expression of connective tissue cell-related genes (COLIA1, LUM, FN1), exclusively distributed within the region displaying consistent morphology observed in the H&E staining.

To assess the transcriptomic capture accuracy of Patho-DBiT in this long-term stored FFPE section, high-plex super-resolution spatial phenotyping was conducted on the adjacent tissue using CODEX technology (Co-Detection by Indexing) (FIG. 3C). The resulting cellular proteomic data depicted the histological profile of the AITL section, showcasing a population of malignant CD4+T follicular helper (Tfh) cells intermixed with a diverse infiltrate of various immune cells. Flow cytometry analysis revealed an abnormal immunophenotype of the Tfh cells: CD3+CD4+CD8-CD7dim+CD5+CD2+CD10+ (data not shown), consistent with the extensive expression of CD4 and the absence of CD8 and Granzyme B in the CODEX data (figure not shown). The expression patterns of B cells (defined by CD19, MS4A1 encoding CD20, CD22, and CD37), T cells (defined by the T cell receptor beta constant region gene TRBC2), and macrophages (defined by LYZ, CHIT1, GPNMB, FTH1) were traced. This was correlated with the surface markers of these cells-CD20 for B cells, CD4 for T cells, and CD68 for macrophages-extracted from the CODEX data. The analysis revealed a strong correlation between the two modalities, evident in their spatial distributions (FIG. 3D). The proliferation marker MKI67 and PDCDI, a marker frequently expressed on malignant AITL cells, exhibited widespread distribution in this section, consistently identified by CODEX (figure not shown).

Analyzing the differentially expressed genes (DEGs) within cluster 0 in comparison to the others, a notable upregulation of B cell markers (CD19, CD22, MS4A1), markers specific to malignant Tfh cells (CXCL13, BCL6, IL21, ICOS, CXCR5, SH2DIA encoding SAP and markers associated with follicular dendritic cells (CR2 encoding CD21, CXCL13, LIF-IL-6 class cytokine) were observed. The detailed profile from the zoomed-in CODEX analysis of this region corroborated the active expression of CD20, CD4, and CD21 (FIG. 3E). This intricate composition, indicative of a distinctive tumor microenvironment, warrants further investigation. Next, cell-cell interactions were explored by examining the expression patterns of ligands and receptors within this region. The analysis highlighted that the most prevalent interaction pair in this network was the communication link between CXCL13 and its receptor genes, including CXCR3, CXCR4, and CXCR5 (FIGS. 3F-3G). Notably, CXCL13 served as a specific diagnostic marker for AITL, given its high expression in nearly all cases, and its interaction with CXCR5 is deeply implicated in tumorigenesis. Patho-DBiT accurately unveiled this regulatory mechanism of AITL, complemented by the spatial distribution profile (figure not shown).

Signaling pathways governed by DEGs within cluster 0 were reviewed. Alongside the activation of numerous fundamental T cell immune functions in this cluster (figure not shown), the analysis pinpointed the upregulation of a cascade of AITL-specific signaling pathways integral to the pathogenesis process, including PI3K/AKT, CD40, JAK/STAT, NF—κB, ICOS-ICOSL, VEGF, mTOR, and ERK/MAPK (FIG. 3H). Conversely, general cancer suppression signals like p53 and PTEN were significantly inhibited within this cluster. Moreover, the signaling of Rho GTPases, known for their functional involvement in lymphoma initiation and progression, showed significant activation within these cells. This activation coincided with upregulated pathways associated with cell cycle regulation and metabolism. Together, the high-sensitivity spatial transcriptomics enabled by Patho-DBiT profiling recapitulated the underlying biology of lymphomagenesis in a 5-year archived AITL tissue. This comprehensive understanding was summarized through a graphical network encompassing canonical pathways, upstream regulators, and biological functions (FIG. 3I).

### Example 4. Genome-Scale Spatial Genetic Variation Profiling for Tumor Discrimination

To further assess the potential utility of Patho-DBIT in aiding pathological evaluation, spatial profiling was conducted on a biopsy from a patient diagnosed with extranodal marginal zone lymphoma of mucosa-associated lymphoid tissue (MALT), a low-grade non-Hodgkin B-cell lymphoma. The section, derived from a FFPE block stored for three years, underwent spatial barcoding using a microfluidic device with a resolution of 50 μm. Biopsy from the gastric antrum of the stomach showed a dense nodular infiltrate of lymphocytes primarily in the mucosa, as detected by endoscopy (figure not shown). The infiltrate was composed of small to intermediate sized lymphocytes with monomorphic ovoid nuclei, condensed chromatin, and a small amount of cytoplasm. There were numerous small mature plasma cells in the lamina propria. The epithelium was predominantly intact and showed no dysplasia, and the lymphoma does not extend to base of biopsy.

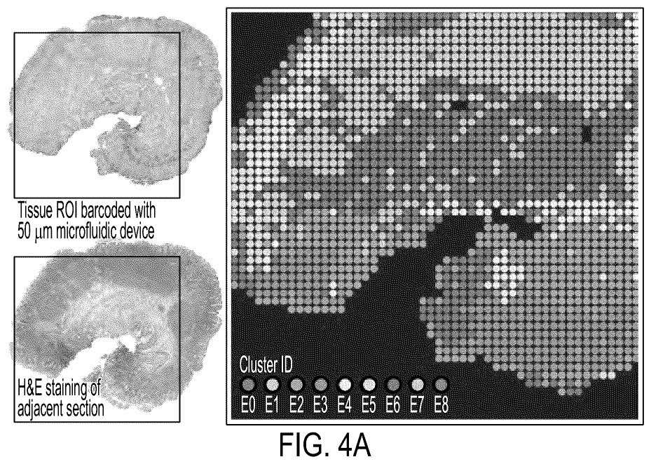

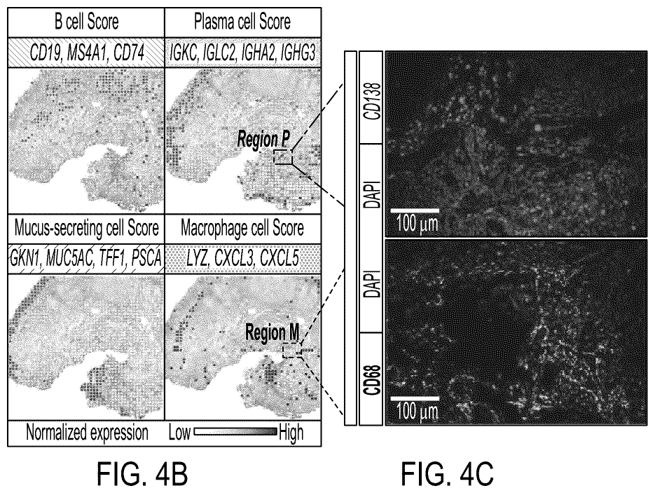

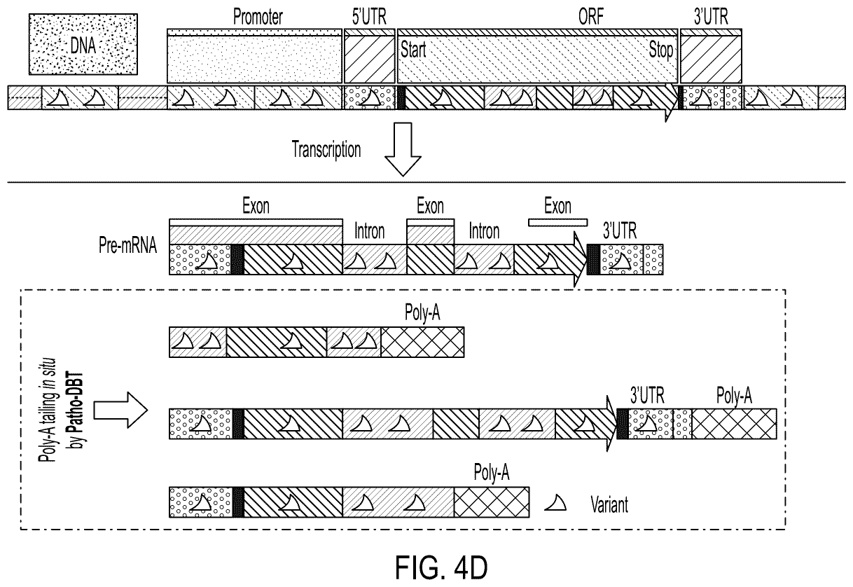

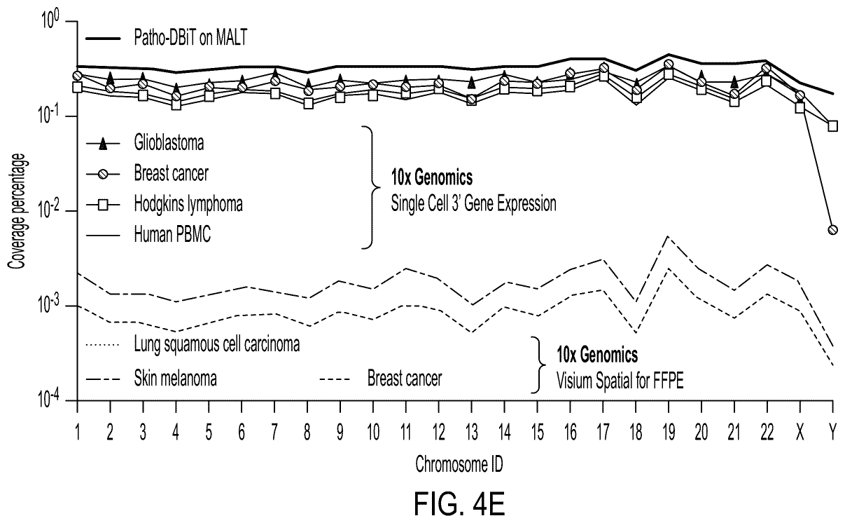

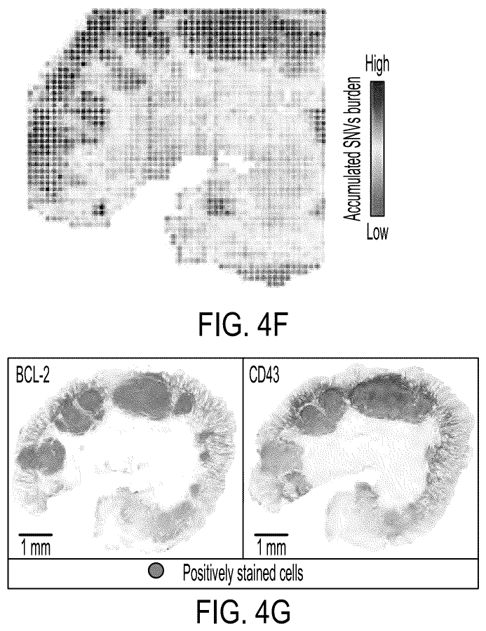

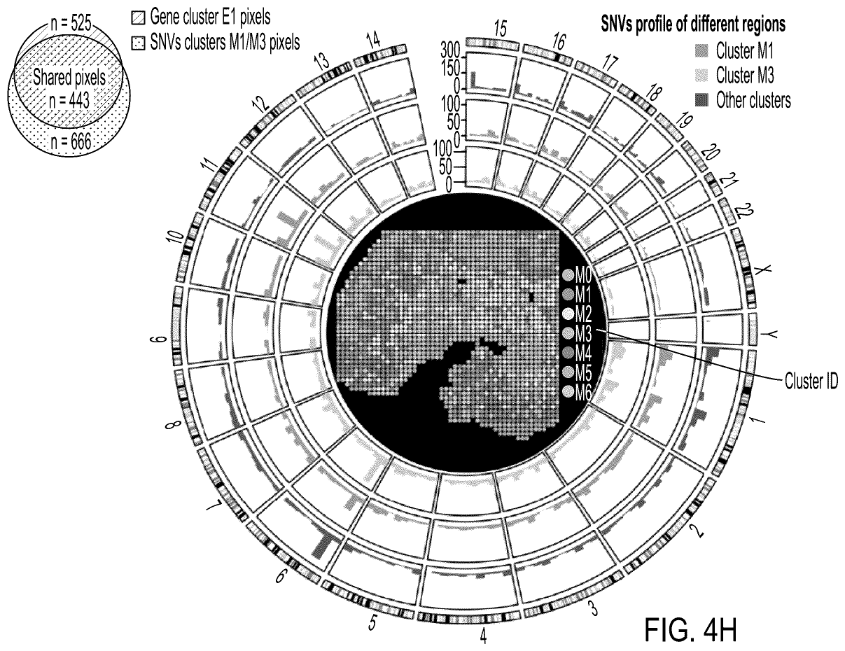

Unsupervised clustering and UMAP visualization revealed 9 clusters that spatially mirrored the histological structures (FIG. 4A). Within these clusters, distinct cell types such as B cells, macrophages, plasma cells, and mucus-secreting cells were delineated based on canonical marker gene expression (FIG. 4B). These cell types were uniquely distributed in Cluster E1, E4, E5, and E6, respectively. A faint expression of the Plasma Cell Score and Macrophage Cell Score was observed in the designated Region P and Region M in FIG. 4B. To validate that this signal reflects actual cellular presence rather than background noise, immunofluorescence (IF) assays targeting CD138 and CD68 phenotypic markers were conducted on adjacent sections (FIG. 4C). The results confirmed the cell identity, providing further support for Patho-DBIT's capability to capture rare cell types in specific regions.

Mutations constantly occur in the precursor mRNA molecule, impacting the final mature mRNA and the encoded protein. By in situ poly(A) tailing of the entire RNA spectrum (FIG. 4D), it was possible that Patho-DBIT could effectively capture variations printed in pre-mRNAs. Given the numerous computational pipelines established for robust mutation detection in scRNA-seq datasets, a comparative analysis of the genomic location coverage bandwidth between Patho-DBIT and 10× Genomics single-cell 3′ gene expression datasets were conducted. Here, the coverage percentage for each chromosome was determined by dividing the number of regions detected in the dataset by the total number of regions with transcripts as per the GENCODE annotation. The spatial data from the FFPE MALT section showed higher coverage capability than scRNA-seq datasets from both human cancer samples and healthy donor peripheral blood mononuclear cells (PBMC), empowering Patho-DBiT to faithfully capture variations (FIG. 4E). This performance is over 176-fold higher than that observed in Visium spatial FFPE datasets from various cancer samples.

To delineate the mutational profile of this section the entire sequencing dataset was aligned to the human genome and implemented a germline variant calling pipeline to identify all potential single nucleotide variations (SNVs). Each spatial pixel and SNV site were assigned a value based on the following criteria: 0 for wild type if no mutated nucleotide was detected, 1 for heterozygous mutation if both mutated and wild-type nucleotides were present, and 2 for homozygous mutation if only mutated nucleotides were detected, resulting in a mutational expression matrix. The spatial expression map of accumulated SNVs highlighted a notably higher mutational burden in the B cell region compared to other areas (FIG. 4F). The tumor signature in these B cells was validated through immunohistochemistry (IHC) staining using canonical markers commonly detected in MALT tumor cells, namely B-cell lymphoma 2 (BCL-2) and CD43, on adjacent sections (FIG. 4G). The expression of these markers exhibited a strong correlation with the spatial SNV profile.

The potential of leveraging RNA-encoded variation information for unsupervised tumor discrimination was assessed. Spatial clustering of the mutational expression matrix revealed 7 subpopulations (FIG. 4H). Cluster M1 and M3 exhibited notable overlap with the tumor region; out of 525 pixels from the tumor B cell-enriched E1 cluster, 443 pixels were assigned to M1 or M3. The UMAP visualization showcased the distinct variation profile of these two clusters compared to the other five (figure not shown). Additionally, principal component analysis (PCA) demonstrated that pixel points from M1 and M3 clusters could be differentiated from the rest using the combination of PCI and the top 5 identified principal components (figure not shown). To determine the variation levels across different mutational clusters, the mutation frequency was calculated within a 10,000 bp genomic region, generating their genomic distribution (figure not shown). The counts of genomic regions with SNV frequency >0.01% were significantly higher in M1 and M3, confirming their elevated mutational levels (figure not shown). To depict the genome-wide distribution of SNVs, somatic variation calling was conducted within the M1 and M3 using pixels from the other five clusters as controls, revealing a chromatin and region-specific pattern of identified SNVs within these two tumor clusters as compared to normal (FIG. 4H). Collectively, these findings suggested that Patho-DBiT possesses the capability to autonomously distinguish tumor from non-tumor regions based on the variation profile.

### Example 5: Spatial Regulatory Network of microRNA-mRNA Interactions in Tumorigenesis

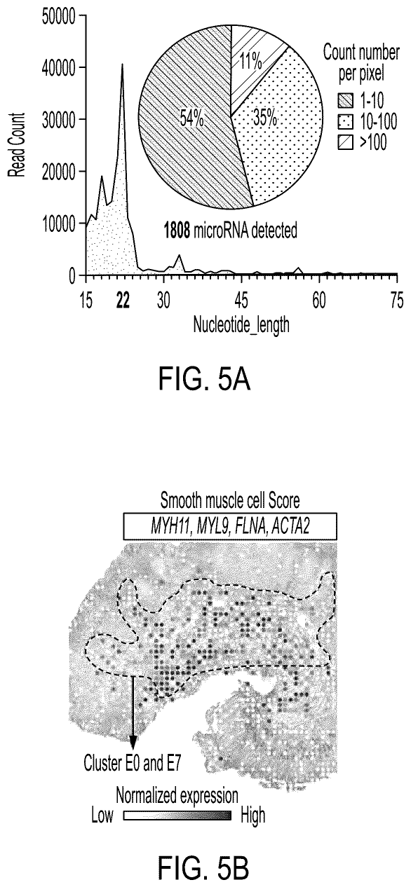

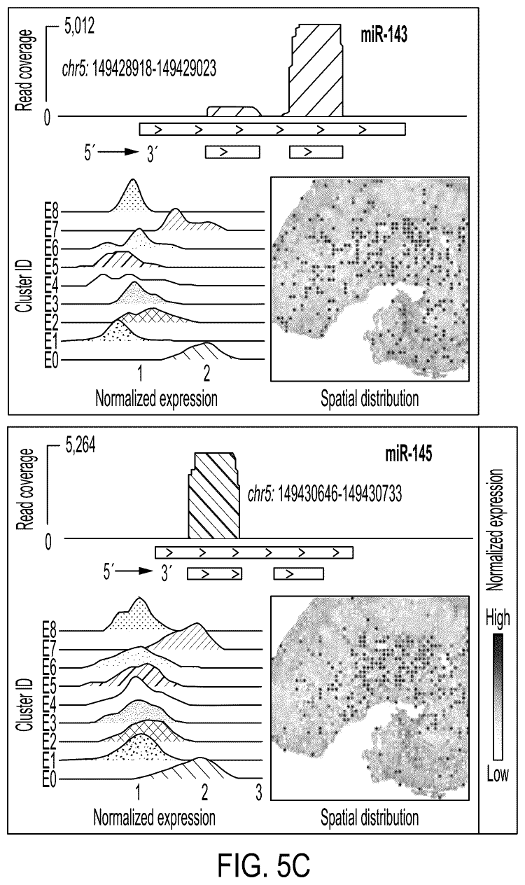

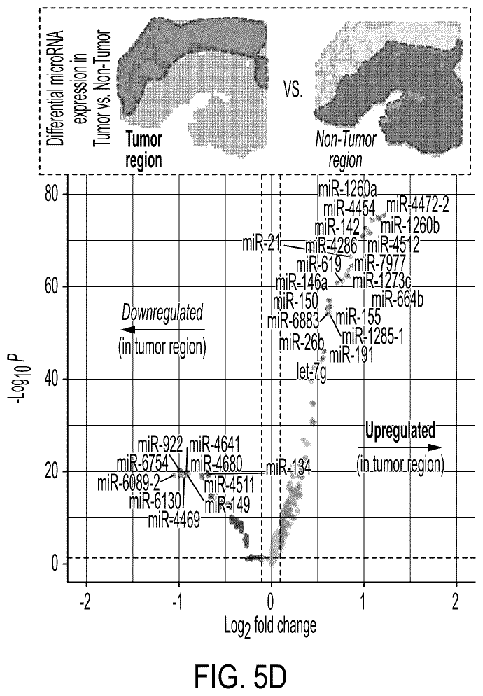

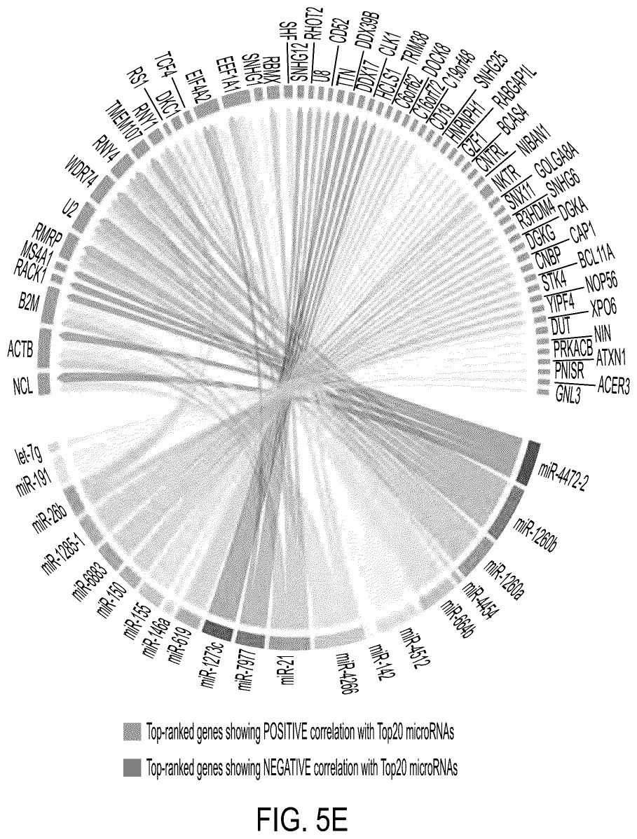

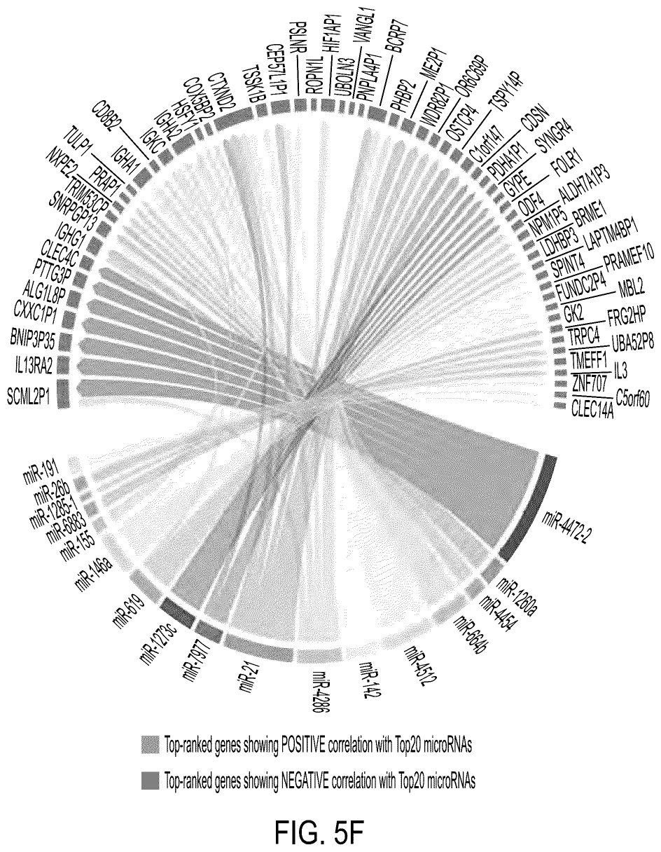

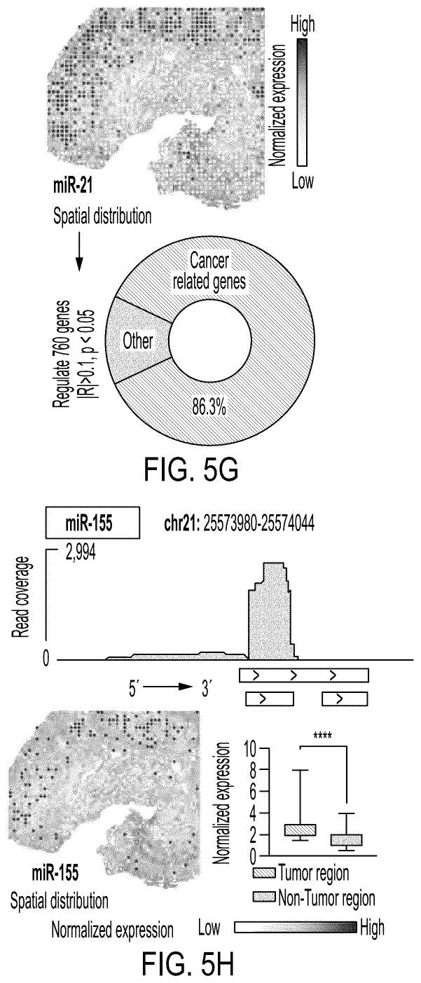

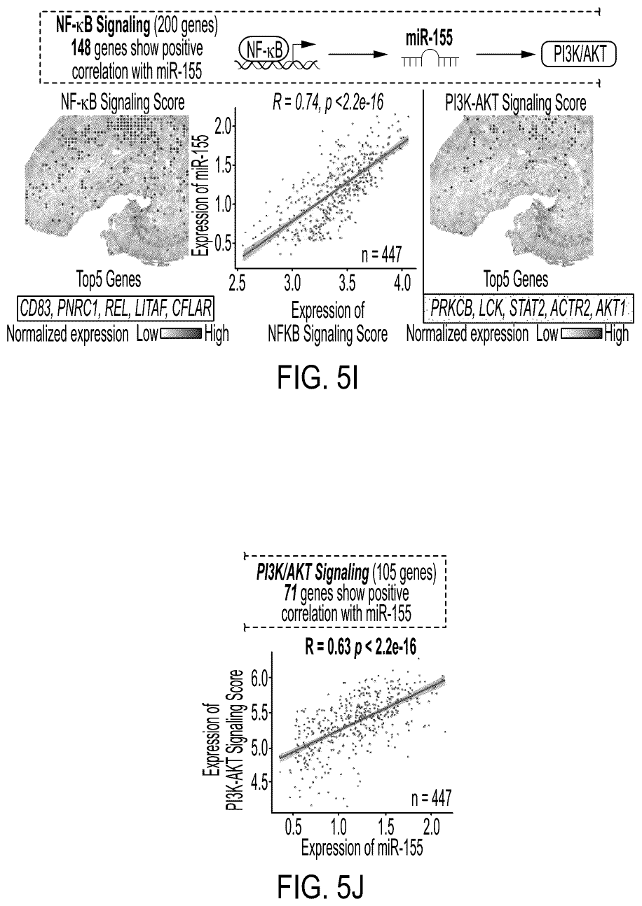

Next, Patho-DBiT's capacity for co-mapping large and small RNAs in clinical samples was assessed with a specific focus on microRNAs that played diverse roles in various pathologies, including cancer. Out of 2300 true human mature microRNAs, Patho-DBIT detected 1808 in the MALT section, with the count of mapped reads accurately peaking at 22 nucleotides in the dataset (FIG. 5A). Assessing the UMI count per pixel for all identified microRNAs, 54% had fewer than 10 UMIs, 35% had 10-100 UMIs, and the remaining 11% had more than 100 UMIs.

The detection accuracy was assessed by examining tissue-specific microRNAs. Based on both tissue morphology and the enriched expression of marker genes MYH11, MYL9, FLNA, ACTA2, cells within clusters E0 and E7 were discerned as smooth muscle cells (FIG. 5B). Two smooth muscle cell-specific microRNAs, miR-143 and miR-145, known for their involvement in proliferation, differentiation, and plasticity, exhibited markedly elevated expression in clusters E0 and E7, with over 5000 reads precisely mapped to the reference genome location (FIG. 5C). Their spatial distributions were prominently evident in the smooth muscle cell region. Several members of the miR-30 family exert regulatory roles across different stages of mature B-cell differentiation. Patho-DBiT successfully detected three of these members, namely miR-30b, miR-30d, and miR-30c, showcasing elevated expression particularly in the B cell cluster E1 or the plasma cell cluster E5 (figure not shown). MiR-142 is necessary for the normal development of marginal zone B cells, while both miR-1546a and miR-150 are upregulated in marginal zone lymphomas. Consistently, a notably high expression pattern of these three microRNAs was observed in the tumor B cell region of this MALT section (figure not shown).

To further elucidate the regulatory role of microRNAs in tumorigenesis, a differential microRNA expression analysis was conducted between the tumor and non-tumor regions. The majority of microRNAs exhibited substantial upregulation in the tumor region, notably including miR-21, a well-characterized cancer-promoting ‘oncomiR’, along with abovementioned lymphoma-specific microRNAs such as miR-142, miR-146a, miR-150, and miR-155 (FIG. 5D). In contrast, miR-134 and miR-149, two microRNAs known to suppress the proliferation and metastasis of multiple cancer cells, were significantly downregulated in the tumor region. Regulatory network analysis of microRNA-RNA interactions in the tumor region revealed positive correlations between the top 20 upregulated microRNAs and multiple genes implicated in lymphomagenesis (FIGS. 5E-5F), including NCL encoding a BCL-2 mRNA binding protein, ACTB and B2M, which are frequently mutated in aggressive B-cell lymphoma, and EEFIAI, potentially contributing to tumor initiation and progression. Conversely, a broad array of genes exhibited negative correlations with these microRNAs, especially miR-21 and miR-4472-2, the latter also being identified for its role in fostering tumor proliferation and aggressiveness. Likewise, interaction analysis was conducted for the top 10 downregulated microRNAs in the tumor region, clucidating how these regulations influence the transcriptomic signatures of tumor B cells (figure not shown). For instance, the negative correlation observed between miR-134 and CHD8, FTL, WNK1, TACCI, CDKL2, and TCF4 implies that these genes play a role in promoting tumorigenesis in the MALT patient.

A detailed regulatory analysis was performed by focusing on representative microRNAs, with miR-21 as the initial example. This microRNA exhibited enriched spatial expression in the tumor region and significantly regulated 760 genes in this sample, with 86.3% of them identified as cancer-related genes according to the Ingenuity Pathway Analysis (IPA) database (FIG. 5G). As an ‘oncomiR’ known for its pivotal role in the initiation and development of various B-cell malignancies, miR-155 exhibited significantly higher expression in the tumor region, accompanied by precise genome location mapping and spatial distribution (FIG. 5H). This microRNA, being a transactivational target of NF-κB, contributes to the promotion of the PI3K-AKT signaling pathway in B-cell lymphoma. Out of 200 genes in the Gene Set Enrichment Analysis (GSEA)-defined NF-κB signaling activation, 154 exhibited a positive correlation with miR-155 (FIGS. 5H5I-5J). Similarly, a positive correlation was identified between miR-155 and genes linked to the activation of PI3K-AKT signaling, with 89 out of 105 genes showing this relationship (FIGS. 5I-5J). The enriched spatial expression of both signaling pathways was pinpointed in the tumor B cell region. The correlation between miR-155 expression and both signaling pathways was calculated across 447 spatial pixels within the tumor region, resulting in Pearson correlation coefficients of 0.74 and 0.63, respectively (p-value <2.2e-16). Taken together, Patho-DBIT enables spatially resolved co-profiling of large and small RNAs, facilitating the robust and precise construction of a microRNA-mRNA regulatory network in the clinical biopsy.

### Example 6: Spatial RNA Splicing Dynamics Reveals the Developmental Trajectory of Tumor Cells

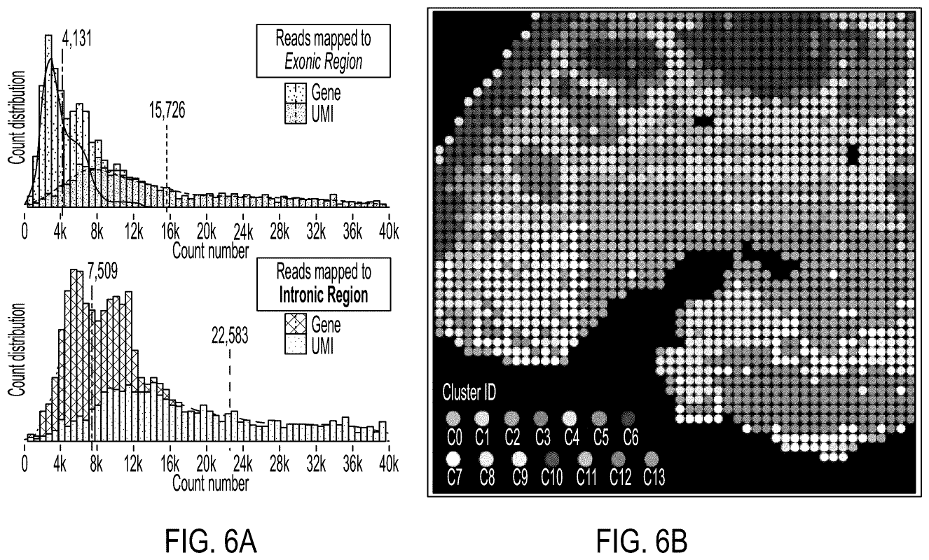

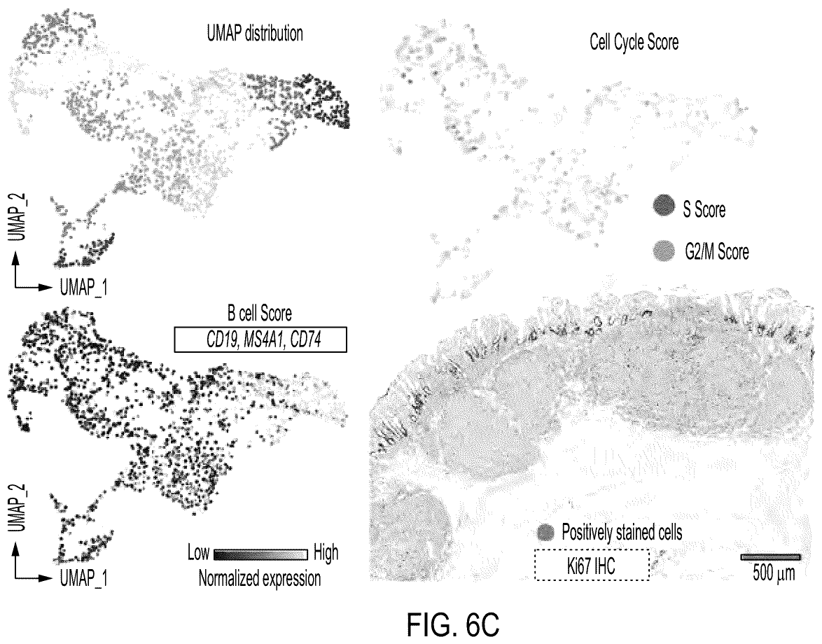

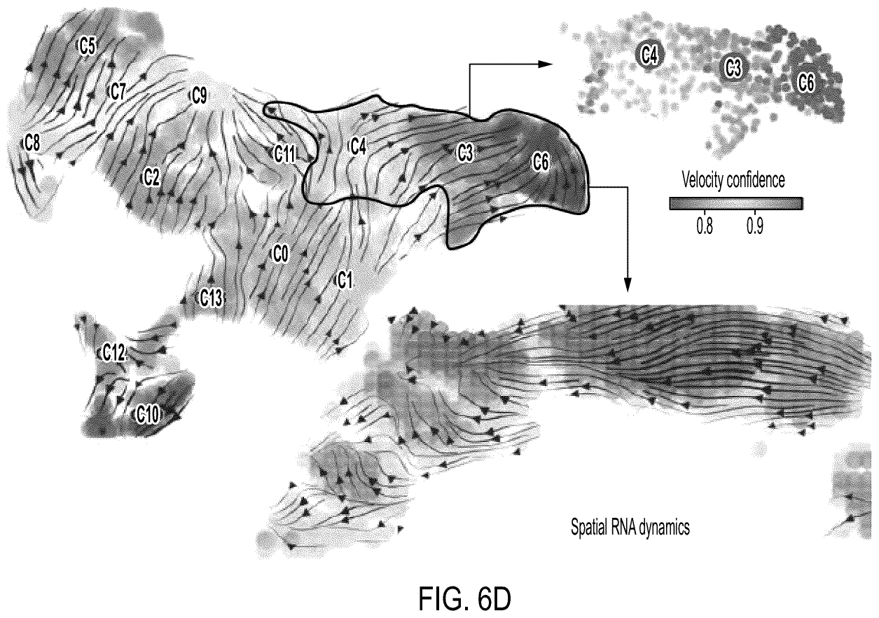

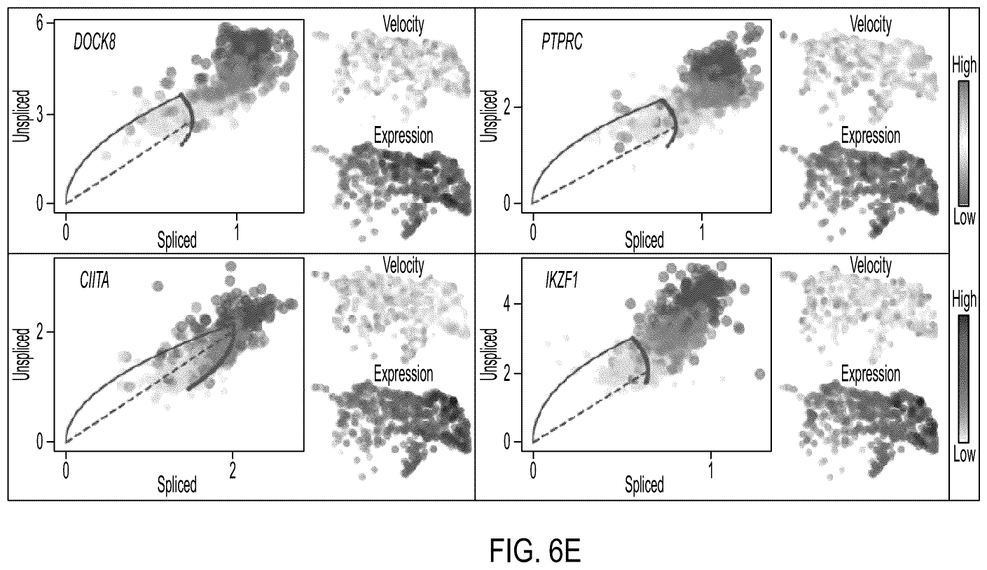

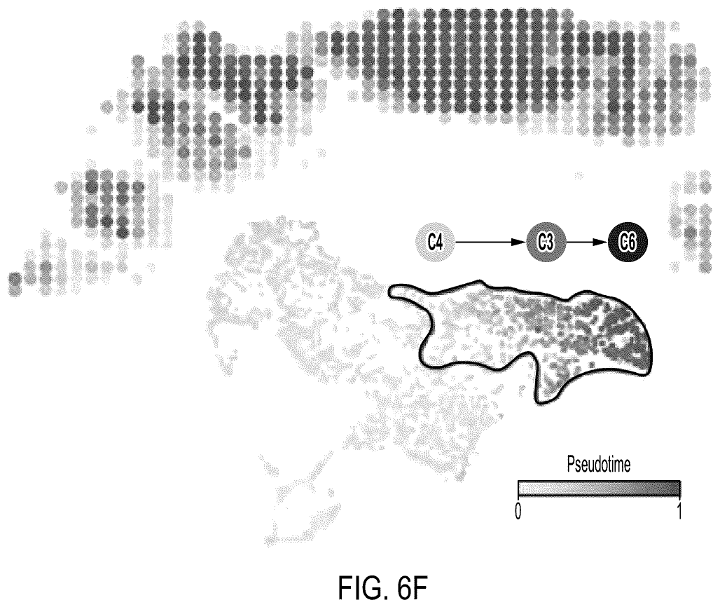

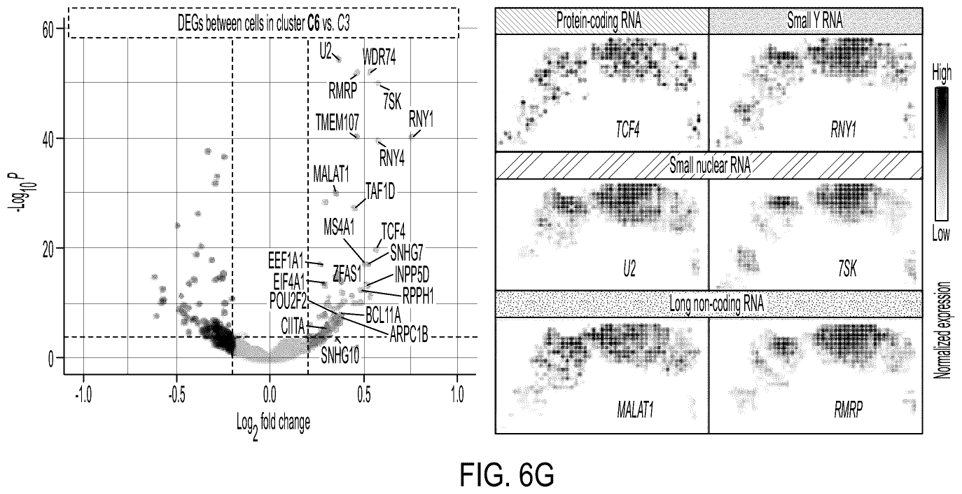

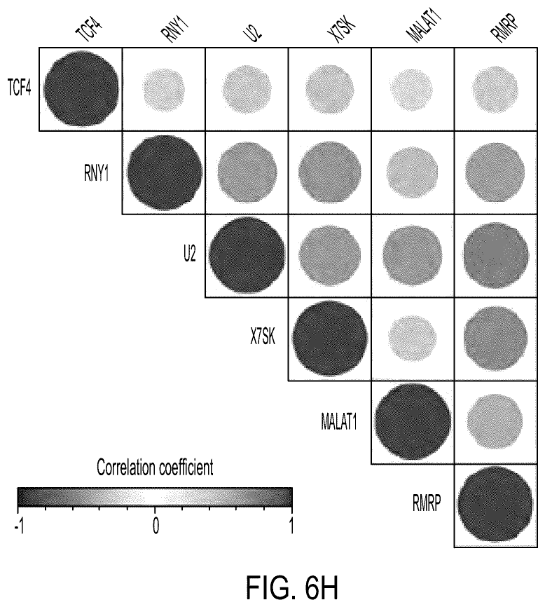

In all the samples, Patho-DBiT primarily includes reads mapped to exonic regions derived from mature spliced transcripts. While these exonic reads yield an average of 4,131 genes and 15,726 UMIs per pixel in the MALT section, a substantial number of intronic molecules in this sample were detected, corresponding to a mean pixel count of 7,509 genes and 22,583 UMIs (FIG. 6A). Without being bound by theory, this observation can be attributed to the poly(A) addition and subsequent capture in the intron regions. By aggregating both the exonic and intronic expression matrices while preserving their individual identities, 14 clusters were identified through unsupervised clustering analysis (FIG. 6B), suggesting that this combined profile could enhance the refinement of intrinsic biological heterogencities of this MALT sample. Particularly, while utilizing solely exonic reads led to the definition of only one B cell cluster (FIG. 4A), the merged matrix identified three clusters (C3, C4, and C6) within this tumor region using identical clustering parameters (FIG. 6B). Their B cell signature was authenticated by an enriched expression of the B cell Score characterized by CD19, MS4A1, and CD74. The three clusters exhibited no prominent variations in cell cycle stages, as evidenced by their uniformly low levels of S and G2/M activities, further confirmed through sparse IHC staining of Ki67 (FIG. 6C). Thus, their inherent distinctions and connections warrant deeper investigation.

RNA velocity enables the reconstruction of dynamic changes in gene expression by leveraging the ratio of newly transcribed, unspliced pre-mRNAs to mature, spliced mRNAs, with the former identifiable through the presence of introns. Typically, bulk or single-cell RNA-seq datasets comprise only 15-25% of reads as unspliced intronic sequences, primarily originating from secondary priming positions within intronic regions. Notably, Patho-DBiT generated a higher proportion of intronic reads compared to the exonic part, enhancing the reliability of RNA velocity analysis. Employing sc Velo, a method that characterize the full transcriptional dynamics of splicing kinetics using a likelihood-based dynamical model, transient cellular states of all the identified clusters were delineated based on the combined matrix. Within the spatially organized tumor B cell region, a developmental trajectory originating from C4 extending towards C6 (FIG. 6D) was observed. Across the majority of pixels, boasting high velocity confidence exceeding 0.9, a spatial RNA splicing dynamics map was constructed for MALT tumor cells, potentially unveiling their differentiation stages as defined by splicing rates. Furthermore, prominent genes that drove the primary processes of dynamic behavior were identified, with the top-ranked genes clearly displaying higher splicing activities in C6 compared to C3 and C4 (FIG. 6E) Among these pivotal driving genes, DOCK8 and PTPRC (also known as CD45) regulated BCR signaling and activation of memory B cells, CIITA, SLC38A1, SYK, and FCRL5 have been identified as prognostic factors for hematological malignancies, IKZFI served as a central regulator of hematopoiesis, and ARHGAP44, a gene encoding Rho GTPase activating protein 44, played an extensive role in lymphoma initiation and progression. All of them exhibited notably higher expression and velocity in cluster C6, indicating a greater level of upregulation and dynamic change compared to the relatively steady state observed in cells from C3 and C4.

Next, it was determined that a universal pseudotime was shared among genes and represented the cell's internal clock. While all the clusters outside the tumor region displayed a static cell fate, changes began to manifest from C4 and progressively intensified toward C6 (FIG. 6F). This spatial directional transition, coupled with the noted higher velocities in C6, further implied that tumor cells in C6 pixels were produced at a later stage. Putative driver genes contributing to this pseudotime trajectory were identified and ordered by their likelihoods across these clusters, among which the top-ranked gene, BCL2, was a key antiapoptotic regulator critical for lymphoma pathogenesis (figure not shown). A DEG analysis was performed between cells in cluster C6 and C3, revealing a regulatory profile consisting of both large and small RNAs (FIG. 6G). Among the significantly upregulated molecules in C6 compared to C3, TCF4 primarily functioned as a transcriptional activator in B cell development and contributes to lymphoma pathogenesis, RNY1 was affiliated with the Y_RNA class and mainly involved in DNA replication and RNA stability, U2 spliceosomal small nuclear RNA (snRNA) was an essential component of the major spliceosomal machinery, 7SK was another snRNA controlling the activity of a major transcription elongation factor P-TEFb, and MALATI and RMRP were long non-coding RNAs that promote the development of various lymphomas. All the small RNAs discussed here were accurately and substantially mapped by Patho-DBiT (figure not shown). The heightened spatial expression of these molecules, along with their significant internal positive correlations (FIG. 6H), further substantiated the increased dynamic activities within the C6 subpopulation. Together, with superior intronic read capture efficiency, Patho-DBiT spatially mapped RNA splicing dynamics associated with the developmental trajectory of tumor B cells.

### Example 7: Mapping Spatial Evolution of Tumor Progression at the Cellular Level

The analysis was extended by spatially mapping fundus nodule biopsy sections using 10 μm microfluidic devices at cellular-level resolution. Collected from the same patient depicted in FIG. 4A at the same time, the diagnosis progressed from low-grade MALT to diffuse large B-cell lymphoma (DLBCL). The biopsy revealed a diffuse sheet of atypical large lymphocytes, extending from glandular structures to the deep margin of the biopsy (figure not shown). Predominantly large and pleomorphic, the tumor cells exhibited irregular nuclear contours, dispersed chromatin, and a moderate amount of cytoplasm, with frequent mitoses. Focal tumor infiltration of epithelium and cosinophilia were observed, while the surface epithelium showed no significant abnormalities.

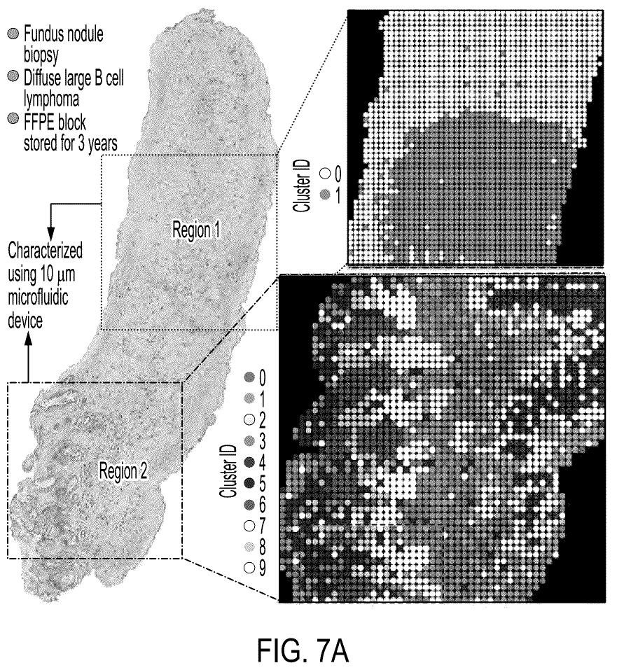

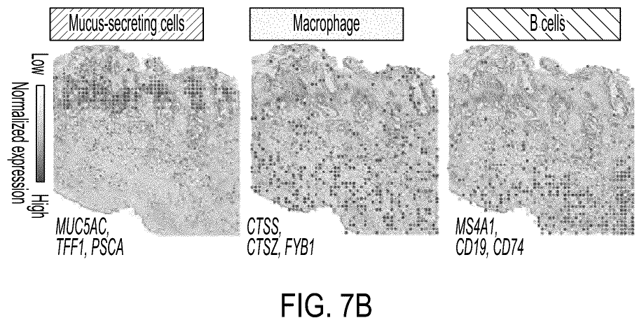

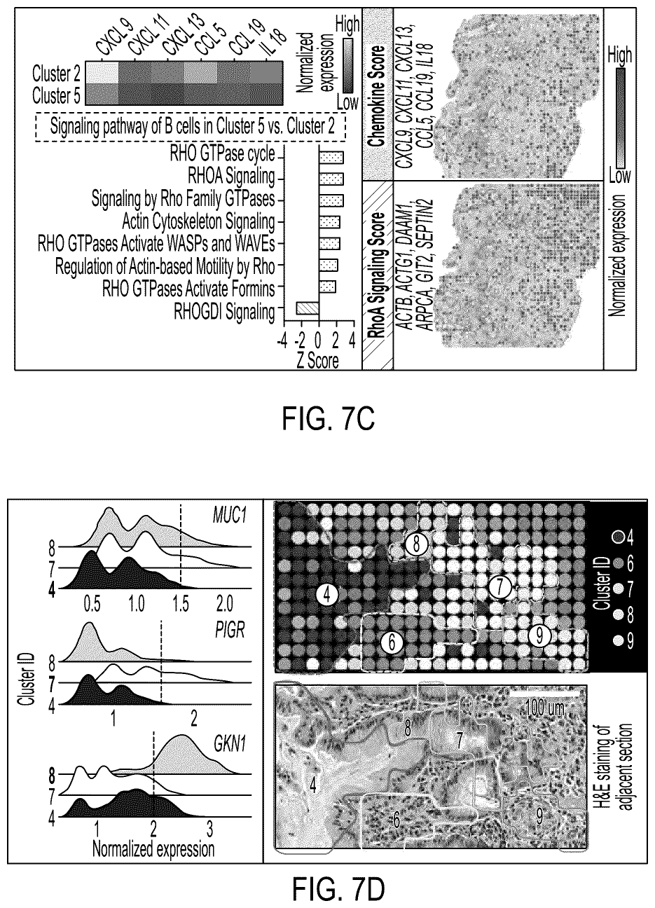

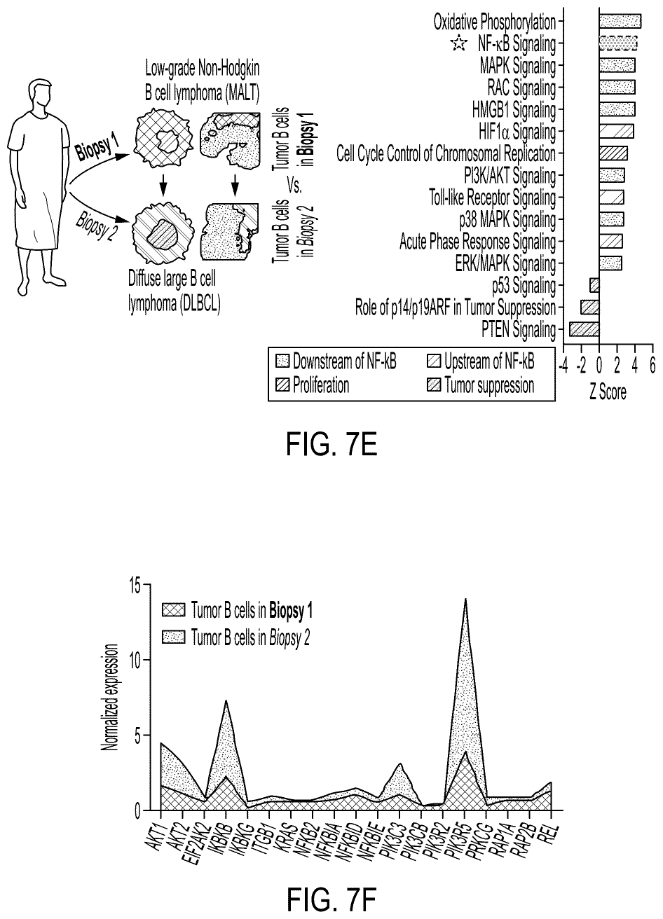

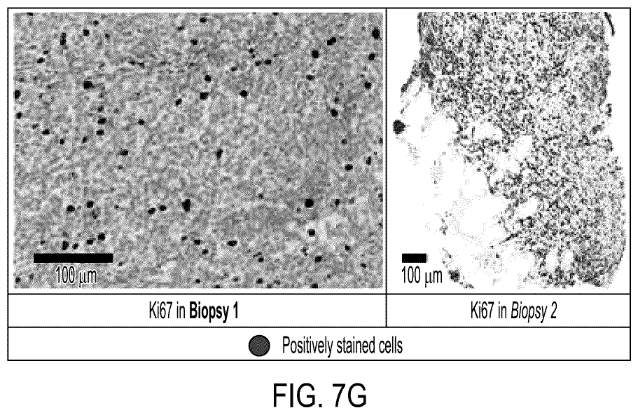

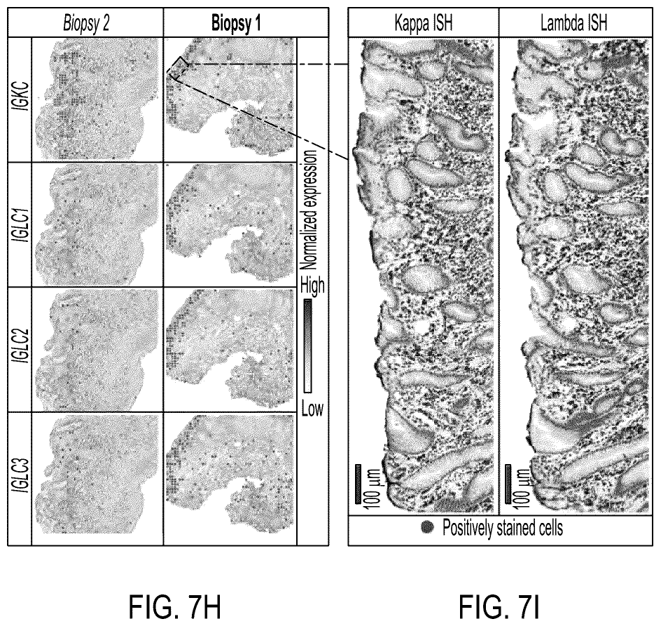

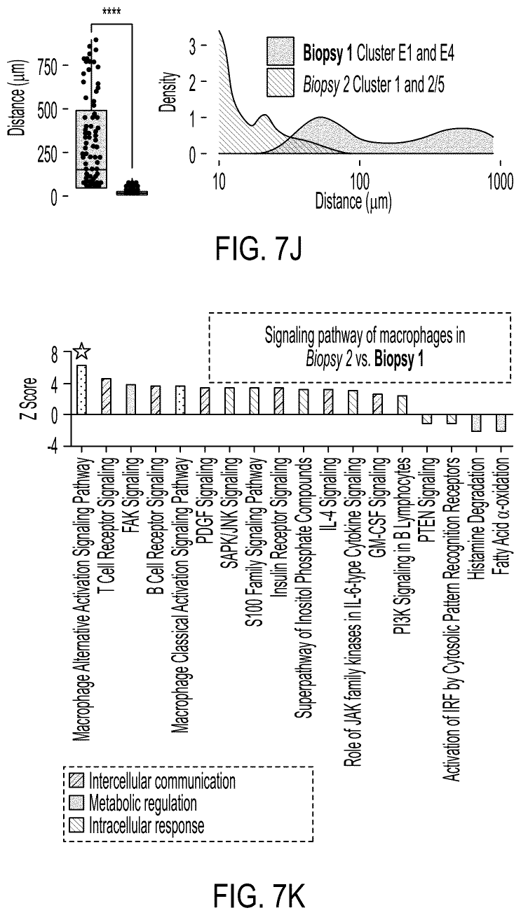

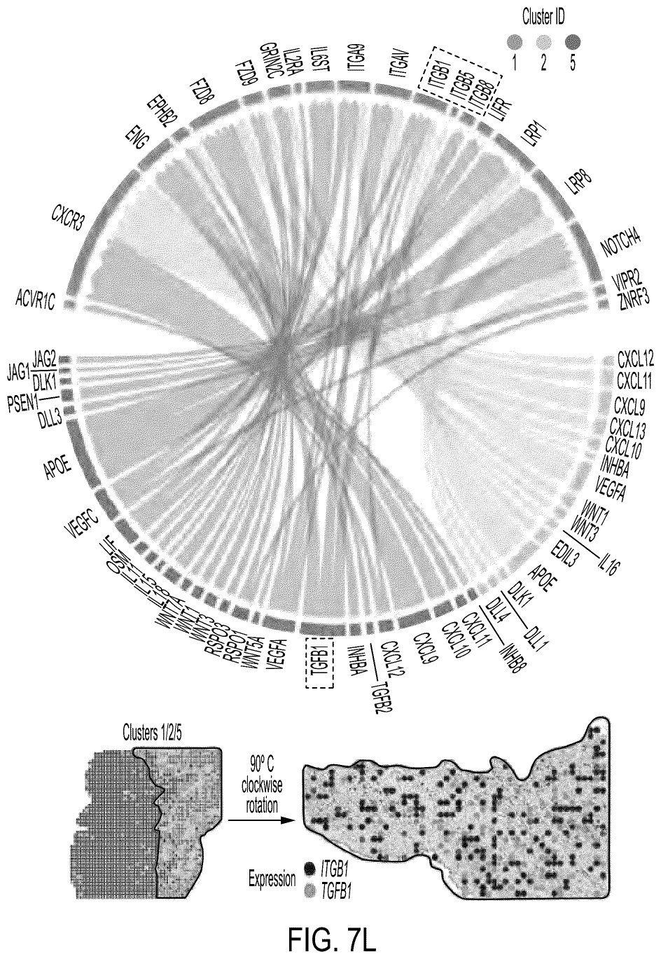

Sections from two distinct regions were selected for spatial barcoding, detecting an exonic average pixel count of 2,292 genes and 6,021 UMIs in Region 1, and 1,507 genes and 3,466 UMIs in Region 2 (FIG. 7A). Unsupervised clustering of Region 1 identified two clusters with similar phenotypes of B cells, distinguished by varying dynamic levels as indicated by the differential small RNA expression of 7SK, RNY1, and RNY3 in cluster 279,81 (figure not shown). Their tumor signature was verified by IHC staining for BCL-2 and CD43 (figure not shown). In Region 2, a more intricate spatial organization of diverse cell types, including B cells, macrophages, and mucus-secreting cells, was identified (FIG. 7B). Notably, Patho-DBIT resolved intrinsic heterogencities among tumor B cells. While both cluster 2 and 5 cells showed enriched expression of B cell markers, cluster 2 displayed enhanced chemokine gene activity (FIG. 7C). These genes engaged in extensive communication with cells in cluster 5 through ligand-receptor pairs (figure not shown), potentially linked to their significant upregulation of Rho GTPases related signaling pathways. Clusters 4, 7, and 8 were characterized as gastric mucus-secreting cells, exhibiting high expression of signature genes including MUC5CA, TFF1, and PSCA. Patho-DBiT unraveled their molecular heterogeneities: cluster 7 showed elevated PIGR expression actively participating in the transcytosis of soluble polymeric isoforms of immunoglobulins, cluster 8 displayed higher GNKI expression related to gastric mucosal inflammation, and cluster 4 had reduced MUCI expression compared to the other two. These subpopulations, along with cluster 6 enriched with plasma cells, form a unique transcriptomic neighborhood revealed by the cellular-level spatial mapping, closely aligning with the tissue morphology defined by H&E staining of an adjacent section (FIG. 7D).

Thoughtfully selected biopsies were collected at the same timepoint to elucidate the spatial molecular dynamics propelling tumor progression. Comparative analysis of gene expression profiles in the tumor region, transitioning from low-grade MALT to high-grade DLBCL, revealed a significant upregulation of NF-κB signaling and its associated upstream and downstream pathways (FIG. 7E). Furthermore, canonical tumor suppression pathways, including p53, p14/p19ARF, and PTEN, exhibited notable inhibition. Elevated expression of a range of key genes involved in the NF-κB signaling pathway was observed, particularly AKTI, IKBKB, PIK3C3, and PIK3R5 (FIG. 7F). Without being bound by theory, the considerable NF-κB activation, a hallmark of aggressive DLBCL, could be associated with their significantly active c-Myc (MYC) signaling, spatially concentrated in the tumor B cell zone (figure not shown). This was confirmed by pronounced c-Myc IHC staining presented in the DLBCL biopsy (figure not shown). A strong positive correlation between MYC signaling and miR-21 expression (figure not shown) was shown, aligning with the documented role of c-Myc in activating miR-21 by directly binding to its promoter. The coordinated upregulation of these signaling pathways, functional molecules, and their interactive networks could contribute to the high proliferative index in the DLBCL section, as indicated by its high Ki67 activity (˜70%) compared to the MALT section (<10%) (FIG. 7G).

The detection of B cell clonality has proven valuable for diagnosing B cell lymphomas. Abnormalities in light chain composition could lead to a significant elevation in one of the chains, resulting in an abnormal kappa: lambda ratio. In the MALT biopsy, a comparable expression level of the kappa chain constant domain gene IGKC and lambda chain genes, including IGLC1, IGLC2, and IGLC3, was observed and confirmed by ISH stain for kappa and lambda mRNA (FIG. 7H). The finding of polytypic light chains in this biopsy is likely due to the indolent stage, where no clone dominates. However, in the progressed DLBCL biopsy, the plasma cells were mostly kappa type, represented by a dominant expression of IGKC, which could be associated with its increased malignancy level. Consistently, a significant upregulation of pathways linking inflammation to cancer and involved in tumor cell survival and malignant progression was observed, alongside a downregulation of principal tumor suppression pathways in the DLBCL plasma cells compared to those in the MALT counterpart (figure not shown). Notably, this patient had been followed for lymphadenopathy until the identification of enlarged lymph nodes 3 years later. This led to biopsy of a 6 cm neck lymph node revealed a recurrence of DLBCL with kappa-restricted monoclonal tumor B cells, providing robust evidence for the detection accuracy and potential diagnostic value of Patho-DBIT.

Finally, the functional profile of macrophages and their spatial interaction with tumor cells in the two biopsies was investigated. In the MALT section, macrophages in cluster E4 remained distantly positioned from the tumor zone in cluster E1 (FIG. 4A). In contrast, a profound infiltration of macrophages (cluster 1) into the tumor region (clusters 2/5) was observed in the DLBCL section (FIG. 7A), resulting in a significantly reduced macrophage-tumor distance in the latter (FIG. 7J). The DEGs between macrophages in DLBCL and MALT regulated a significant activation of the macrophage alternative activation signaling pathway (figure not shown). Additionally, associated pathways involved in intercellular communications, metabolic modulations, and intracellular responses were affected, potentially contributing to the roles of tumor-associated macrophages in promoting tumor growth and invasion. The deeper exploration of ligand-receptor interactions shed light on the communication network between macrophages and tumor B cells. This analysis unveiled a plethora of crucial pairs activated within the DLBCL microenvironment (FIG. 7L), highlighting the potential role of APOE-LRP1 pair in metabolic shaping the conversion from classic (M1) to alternative (M2) macrophage, CXCL9-CXCR3 axis in orchestrating the recruitment of effector T cells, and VEGFC-IL6ST pair in fostering tumor lymphangiogenesis. A noteworthy interaction was the crosstalk between TGF-β (TGFB1) and the integrin family (ITGB1, ITGB5, and ITGB8), potentially instigating the formation of pro-tumorigenic M2-macrophages and facilitating tumor immune escape. The spatial expression pattern of this interaction could be distinctly visualized. Collectively, the high-sensitivity Patho-DBIT was able to spatially map the molecular evolution from low-grade to high-grade tumor at cellular level resolution, deepening the understanding of the complex interplay shaping the tumor microenvironment in DLBCL.

### Example 8: Materials and Methods related to Examples 1-7

### Patient Specimens

De-identified archival formalin-fixed paraffin-embedded (FFPE) human lymphoma tissue blocks, originally collected by physicians for diagnostic purposes, were sourced from the Yale Pathology Tissue Services (YPTS), a Pathology-based Central Tissue Resource Lab that provides comprehensive tissue-related services and materials in de-identified format for investigators at Yale University. The tissue collection was conducted with Yale University Institutional Review Board approval with oversight by Tissue Resource Oversight Committee. Written informed consent for participation, including cases where identification was collected alongside the specimen, was obtained from patients or their guardians, adhering to the principles of the Declaration of Helsinki. Each sample was handled in strict compliance with HIPAA regulations, University Research Policies, Pathology Department diagnostic requirements, and Hospital by-laws. The excisional biopsy from the left upper arm subcutaneous nodule was collected and embedded in 2018 from a patient presenting with angioimmunoblastic T-cell lymphoma (AITL) in multiple lymph nodes and subcutaneous sites. Biopsies from the gastric antrum revealing marginal zone lymphoma of mucosa-associated lymphoid tissue (MALT) and the fundus nodule indicating diffuse large B-cell lymphoma (DLBCL) were collected and embedded in 2020. These biopsies were obtained from a patient who incidentally presented with retroperitoneal lymphadenopathy during imaging originally performed for an orthopedic visit. Upper endoscopy revealed multiple areas of erosion in the stomach, and a breath test for H. pylori was positive.

### Surgical Pathology Report of the Lymphoma Biopsies

The AITL sections showed a sheet of lymphocytes, some with atypical morphology. There were thick and thin bands of fibrosis and interspersed blood vessels. The atypical cells had irregular to round nuclei, speckled chromatin, variable small nucleoli, and a small amount of cytoplasm. There was infiltration into the adjacent fat. Significant mitotic figures, apoptotic figures, or necrosis was not identified. The atypical cells were CD3-positive T cells that are positive for CD4, CD2, CD5, CD10, CXCL13, and PD-1. They were negative for CD25 and CD8 with partial loss of CD7. There were abundant background CD20-postive B cells. The Ki-67 proliferation index was overall approximately 20-30%. T cell gene rearrangement was positive, showing the same peaks as other sites of involvement. Flow cytometric analysis revealed CD4+ T cells were increased in the specimen, representing about 36% of total lymphocytes with few CD8+elements detected. In addition, CD4+ T cells possessed an abnormal immunophenotype.

The MALT sections revealed gastric antral mucosa with numerous lymphoid follicles showing monotonous small lymphocytes that demonstrate ovoid nuclei, condensed chromatin, and indistinct nucleoli. No large cell component was seen in this part. The tumor cells were CD20 positive B cells that co-express BCL-2 and CD43, are negative for CD5, CD10, BCL-6, CD23, LEF1, and cyclinD1. Ki-67 is low at <10%. CD3 highlights scattered small T cells. H. pylori immunostaining was negative.

The DLBCL sections revealed sheets of large pleomorphic lymphocytes, some with horseshoe shaped nuclei, dispersed chromatin, prominent nucleoli, and moderate amount of eosinophilic cytoplasm. There were numerous eosinophils in the background and no substantial small cell lymphoma. The tumor cells were positive for CD20, CD43, and MUMI and negative for CD10, cyclinD1, and CD30. BCL-6 was faintly expressed in <20% of cells. C-myc is expressed in >80% of tumor cells and BCL-2 was expressed in >70% of cells. Ki-67 proliferation index was approximately 70%. CD3 positive small T cells are scattered. Para-aortic lymph node biopsy performed simultaneously showed involvement by metastatic DLBCL.

### Mouse Paraffin Tissues

The mouse E13 embryo, caudal hippocampus coronal brain/Region.9, and lymph node sections were purchased from Zyagen (San Diego, CA). Tissues were freshly harvested from C57BL/6 mice fixed in 10% Neutral Buffered formalin and processed for embedding in low temperature melting paraffin. All tissue preparation steps from harvesting to embedding in paraffin were done in RNase-, DNase-, and protease-free conditions. Tissue sections were hematoxylin and eosin (H&E) stained and examined by histologists with extensive experience to be sure of excellent morphology and high quality.

### Sample Handling and Section Preparation

For both human and mouse samples, paraffin blocks were sectioned at a thickness of 7-10 μm and mounted on the center of Poly-L-Lysine coated 1×3″ glass slides. Serial tissue sections were collected simultaneously for Patho-DBiT and other staining. The sectioning of lymphoma patient samples was carried out at YPTS, while mouse sectioning was performed by Zyagen technicians. Paraffin sections were shipped in tightly closed slide boxes or slide mailers at room temperature and stored at −80° C. upon receipt until use.

### Fabrication of Microfluidic Device

The comprehensive fabrication process, employing standard soft lithography, has been detailed in a previous publication by the inventors. See Su, G., Qin, X., Enninful, A., Bai, Z., Deng, Y., Liu, Y., and Fan, R. (2021). Spatial multi-omics sequencing for fixed tissue via DBiT-seq. Star Protoc. 2, 100532, which is incorporated herein by reference in its entirety. Briefly, high-resolution chrome photomasks with a customized pattern were printed and ordered from Front Range Photomasks (Lake Havasu City, AZ). Upon receipt, the masks underwent cleaning with acetone to remove any dirt or dust. Master wafers were then produced using SU-8 negative photoresist (SU-2010 or SU-2025) on silicon wafers following the manufacturer's guidelines, with feature width of 50 μm, 20 μm, or 10 μm. The newly fabricated wafers were treated with chlorotrimethylsilane for 20 minutes to develop high-fidelity hydrophobic surfaces. Subsequently, polydimethylsiloxane (PDMS) microfluidic chips were fabricated through a replication molding process. The base and curing agents were mixed thoroughly with a 10:1 ratio following the manufacturer's guidelines and poured over the master wafers. After degassing in the vacuum for 30 minutes, the PDMS was cured at 70° C. for at least 2 hours. The solidified PDMS slab was cut out, and the inlets and outlets were punched for further use.

### DNA Barcodes Annealing

DNA oligos used in this study were procured from Integrated DNA Technologies (IDT, Coralville, IA) and the sequences were listed. Barcode (100 μM) and ligation linker (100 μM) were annealed at a 1:1 ratio in 2×annealing buffer (20 mM Tris-HCl pH 8.0, 100 mM NaCl, 2 mM EDTA) with the following PCR program: 95° C. for 5 minutes, slow cooling to 20° C. at a rate of−0.1° C./s, followed by 12° C. for 3 minutes. The annealed barcodes can be stored at −20° C. until use.

### Tissue Deparaffinization and Decrosslinking

Tissue section was retrieved from the −80° C. freezer and equilibrated to room temperature for 10 minutes until all moisture dissipated. Following this, the tissue slide underwent a 1-hour baking process at 60° C. to facilitate softening and melting of the paraffin. Removal of paraffin was achieved by immersing slides in Xylene for two changes, followed by rehydration in a series of ethanol dilutions, including two rounds of 100% ethanol and once each of 90%, 70%, and 50% ethanol, culminating in a final wash with distilled water. Each step was performed for a duration of 5 minutes. Subsequently, the tissue slide was submerged in 1×antigen retrieval buffer and subjected to steaming using boiling water for 30 minutes, followed by a 30-minute cooldown to room temperature. After a brief dip in distilled water, intact tissue scan was captured using a 10× objective on the EVOS M7000 Imaging System.

### Permeabilization, in Situ Polyadenylation, and Reverse Transcription

The tissue was permeabilized for 20 minutes at room temperature with 1% Triton X-100 in DPBS, followed by 0.5×DPBS-RI (1×DPBS diluted with nuclease-free water, 0.05 U/μL RNase Inhibitor) wash to halt permeabilization. The tissue slide was then air-dried and equipped with a PDMS reservoir covering the region of interest (ROI). In situ polyadenylation was performed using E. coli Poly(A) Polymerase. Initially, samples were equilibrated by adding 100 μL wash buffer (88 μL nuclease-free water, 10 μL 10× Poly(A) Reaction Buffer, 2 μL 40 U/μL RNase Inhibitor) and incubating at room temperature for 5 minutes. Following wash buffer removal, 60 μL of the Poly(A) enzymatic mix (38.4 μL nuclease-free water, 6 μL 10× Poly(A) Reaction Buffer, 6 μL 5U/μL Poly(A) Polymerase, 6 μL 10 mM ATP, 2.4 μL 20 U/μL SUPERase. In RNase Inhibitor, 1.2 μL 40 U/μL RNase Inhibitor) was added to the reaction chamber and incubated in a humidified box at 37° C. for 30 minutes. To remove excessive reagents, the slide was dipped in 50 mL DPBS and shake-washed for 5 minutes after the reaction. Subsequently, 60 μL of the reverse transcription mix (20 μL 25 UM RT Primer, 16.3 μL 0.5× DPBS-RI, 12 μL 5× RT Buffer, 6 μL 200U/μL Maxima H Minus Reverse Transcriptase, 4.5 μL 10 mM dNTPs, 0.8 μL 20 U/μL SUPERase. In RNase Inhibitor, 0.4 L 40 U/μL RNase Inhibitor) was loaded into the PDMS reservoir and sealed with parafilm. The sample was incubated at room temperature for 30 minutes and then at 42° C. for 90 minutes, followed by a 50 mL DPBS wash as described before.

Spatial Barcoding with Microfluidic Devices

To ligate barcode A in situ, the first PDMS device was meticulously positioned atop the tissue slide, aligning the 50 center channels over the ROI. The chip was imaged to record the positions for downstream alignment and analysis. Afterwards, an acrylic clamp was applied to firmly secure the PDMS to the slide, preventing any inter-channel leakage. The ligation mix, comprising 100 μL 1× NEBuffer 3.1, 61.3 μL nuclease-free water, 26 μL 10× T4 ligase buffer, 15 μL T4 DNA ligase, 5 μL 5% Triton X-100, 2 μL 40 U/μL RNase Inhibitor, and 0.7 μL 20 U/μL SUPERase. In RNase Inhibitor, was then prepared. For the barcoding reaction, 5 μL of the ligation solution, containing 4 μL ligation mix and 1 μL 25 μM DNA barcode A (A1-A50), was introduced into each of the 50 inlets. The solution was withdrawn to flow through the entire channel using a delicately adjusted vacuum. After a 30-minute incubation at 37° C., the PDMS chip was removed, and the slide was washed with 50 mL DPBS. Subsequently, the second PDMS device, featuring 50 channels perpendicular to the first PDMS, was attached to the ROI on the air-dried slide. A bright-field image was captured, and the ligation of barcode B set was performed similarly. Finally, after five flow-washes with 1 mL nuclease-free water to remove residual salt, the final scan was conducted to record the microchannel marks imprinted onto the tissue ROI.

Tissue Lysis and cDNA Extraction

The barcoded tissue ROI was enclosed with a clean PDMS reservoir and securely clamped using acrylic chips. A 2× lysis buffer was prepared in advance, consisting of 20 mM Tris-HCl pH 8.0, 400 mM NaCl, 100 mM EDTA, and 4.4% SDS. For tissue digestion, 70 μL of the lysis mix (30 μL 1× DPBS, 30 μL 2× lysis buffer, 10 μL 20 μg/μL Proteinase K solution) was loaded into the PDMS reservoir, sealed with parafilm, and incubated in a humidified box at 55° C. for 2 hours. After the reaction, the parafilm was removed, and all the liquid containing cDNA was collected into a 1.5 mL DNA low-bind tube. Additionally, 40 μL of fresh lysis mix was loaded into the reservoir to collect any remaining cDNA material. The tissue lysate was incubated overnight at 55° C. to completely reverse crosslinks, after which it could be stored at −80° C. until the subsequent steps.

cDNA Purification, Template Switch, and PCR Amplification

To inhibit Proteinase K activity, 5 μL of 100 μM phenylmethylsulfonyl fluoride (PMSF) in ethanol was introduced into the lysate and incubated at room temperature for 10 minutes with rotation. Following this, ˜35 μL of nuclease-free water was added to adjust the total volume to 150 μL. The cDNA was purified using 40 μL of Dynabeads MyOne Streptavidin CI beads resuspended in 150 μL of 2× B&W buffer (10 mM Tris-HCl pH 7.5, 1 mM EDTA, 2 M NaCl). The mixture was incubated at room temperature for 60 minutes with rotation to ensure sufficient binding, followed by magnetic separation and two washes with 1× B&W buffer with 0.05% Tween-20, and an additional two washes with 10 mM Tris-HCl pH 7.5 containing 0.1% Tween-20. Streptavidin beads bound with cDNA molecules were then resuspended in 200 μL of TSO Mix (75 μL nuclease-free water, 40 μL 5× RT buffer, 40 μL 20% Ficoll PM-400, 20 μL 10 mM dNTPs, 10 μL 200U/μL Maxima H Minus Reverse Transcriptase, 5 μL 40 U/μL RNase Inhibitor, 10 μL 100 μM TSO Primer). The template switch reaction was conducted at room temperature for 30 minutes and then at 42° C. for 90 minutes with gentle rotation. After a single wash with 10 mM Tris-HCl pH 7.5 containing 0.1% Tween-20 and another wash with nuclease-free water, the beads were resuspended in 200 μL of PCR Mix (100 μL 2× KAPA HiFi HotStart ReadyMix, 84 μL nuclease-free water, 8 μL 10 μM PCR Primer 1, 8 μL 10 μM PCR Primer 2). This suspension was then distributed into PCR stripe tubes. An initial amplification was conducted with the following PCR program: 95° C. for 3 minutes, cycling five times at 98° C. for 20 seconds, 63° C. for 45 seconds, 72° C. for 3 minutes, followed by an extension at 72° C. for 3 minutes and 4° C. hold.

Following magnetic removal of the beads, 19 μL of the PCR solution was combined with 1 μL 20× EvaGreen for quantitative real-time PCR (qPCR) analysis using the same program. The remaining samples underwent further amplification, with the cycle numbers determined by ½ of the saturated signal observed in qPCR results. The PCR product was then purified using SPRIselect beads at a 0.8× ratio, adhering to the standard manufacturer's instructions. The resulting cDNA amplicon underwent analysis using a TapeStation system with D5000 DNA ScreenTape and reagents. This stage provided a secure stopping point, allowing the sample to be stored at −20° C. until the next steps.

rRNA Removal, Library Preparation, and Sequencing

The SEQuoia RiboDepletion Kit was employed to eliminate fragments derived from rRNA and mitochondrial rRNA from the amplified cDNA product, following the manufacturer's guidelines. Based on the TapeStation readout profile, 20 ng of cDNA was used as the input amount, and three rounds of depletion were performed. Subsequently, 7 cycles of the aforementioned PCR program were executed to directly ligate sequencing primers, using a 100 μL system consisting of 50 μL 2× KAPA HiFi HotStart ReadyMix, ˜42 μL solution from the rRNA removal step, 4 μL 10 μM P5 Primer, and 4 μL 10 μM P7 Primer. The resulting library underwent purification using SPRIselect beads at a 0.8× ratioprior to being sequenced on an Illumina NovaSeq 6000 Sequencing System with a paired-end 150 bp read length.

### CODEX Spatial Phenotyping Using PhenoCycler-Fusion

Spatial high-plex phenotyping of the adjacent FFPE section was performed following the CODEX PhenoCycler-Fusion user guide (akoyabio.com/wp-content/uploads/2021/01/CODEX-User-Manual.pdf). Briefly, the tissue section underwent deparaffinization, hydration, antigen retrieval and equilibration in staining buffer, followed by antibody cocktail staining incubated at room temperature for 3 hours in a humidity chamber. After the completion of the incubation, a series of sequential steps, including post-fixation, ice-cold methanol incubation, and a final fixative step, were performed. The tissue section, attached to the flow cell, was then incubated in 1× PhenoCycler buffer with additive for a minimum of 10 minutes to enhance adhesion. Afterwards, the CODEX cycles were configured, the reporter plate was prepared and loaded, and the imaging process commenced. Upon completion of the imaging cycles, a final QPTIFF file was generated, which could be visualized using QuPath V0.5.0. Information about PhenoCycler antibody panels, experimental cycle design, and reporter plate volumes can be found.

### H&E, Immunohistochemistry (IHC) and In Situ Hybridization (ISH)

Histological H&E staining and clinical-level IHC and ISH on adjacent FFPE sections were conducted at Yale University School of Medicine, Department of Pathology and at YPTS. These procedures adhered to Clinical Laboratory Improvement Amendments (CLIA)-certified laboratory protocols as well as YPTS's rigorous standard protocols, ensuring precision and accuracy in the analysis of tissue samples.

### Immunofluorescence Staining (IF)

The adjacent FFPE sections underwent a standard IF procedure. After deparaffinization and antigen retrieval, the tissue sections were fixed in 4% formaldehyde for 10 minutes and subsequently blocked with DPBS containing 5% bovine serum albumin for 1 hour at room temperature. CD68 antibodies, diluted at 1:100 in the blocking buffer, were applied and left to incubate overnight at 4° C. Secondary antibodies for CD68, Alexa-594 labeled CD138, and Alexa-647 labeled CD20 were then introduced following a standard IF protocol, with a 30-minute incubation at room temperature. The nuclei were counterstained with DAPI at a 1:4000 dilution. Imaging was conducted using a Leica TCS SP5 Confocal microscope.

### Sequence Alignment and Generation of Gene Expression Matrix

To decode sequencing data, the FASTQ file Read 2 underwent processing, involving the extraction of unique molecular identifiers (UMIs) and spatial Barcode A and Barcode B. The Read 1 containing cDNA sequences was trimmed using Cutadapt V3.4 and then aligned to either the mouse GRCm38-mm10 or human GRCh38 reference genome using STAR V2.7.7a. Utilizing ST_Pipeline V1.7.6, spatial barcode sequences were demultiplexed based on the predefined coordinates of the microfluidic channels and ENSEMBL IDs were converted to gene names, generating the gene-by-pixel expression matrix for downstream analysis. Matrix entries corresponding to pixel positions devoid of tissues were excluded.

### Gene Data Normalization and Unsupervised Clustering Analysis

Spatial gene expression analysis was conducted through the Seurat V4 pipeline. First, SCTransform, designed for normalization and variance stabilization in single-cell RNA sequencing (scRNA-seq) datasets, was employed to normalize gene expression within each pixel. Linear dimensional reduction was performed using the “RunPCA” function, and the optimal number of principal components for subsequent analysis was determined through a heuristic method, generating an ‘Elbow plot’ that ranks PCA components based on their percentage of variances. Second, “FindNeighbors” function was utilized to embed pixels in a K-nearest neighbor graph structure based on the Euclidean distance in PCA space, and “FindClusters” was implemented using a modularity optimization technique to cluster the pixels. Finally, the non-linear dimensional reduction function “RunUMAP” was applied to visually explore spatial heterogeneities using the Uniform Manifold Approximation and Projection (UMAP) algorithm, and the identification of differentially expressed genes (DEGs) defining each cluster was accomplished through the “FindMarkers” function for pairwise comparison between groups of pixels.

Integration with scRNA-Seq Datasets

At a resolution of 50 μm, Patho-DBiT assay pixels captured the expression profiles of multiple cells. The ‘anchor’-based integration workflow employed into Seurat V4 to deconvolute each spatial voxel, predicting the underlying composition of cell types. This facilitated the probabilistic transfer of annotations from a reference to a query set. After standard “SCTransform” normalization of both Patho-DBIT and reference scRNA-seq data, the

“FindTransferAnchors” function identified anchors between the reference scRNA-seq and the query Patho-DBiT object. Subsequently, the “TransferData” function was applied for label transfer, providing a probabilistic classification for each spatial pixel based on well-annotated scRNA-seq identities. These predictions were added as a new assay to the Patho-DBiT object. Unsupervised clustering was then performed on the combined Patho-DBIT and reference dataset, resulting in an integrated UMAP where Patho-DBiT pixels were projected onto the scRNA-seq cluster landscape. The mouse organogenesis reference dataset was obtained from GSE119945, and the mouse brain cortex and hippocampus reference dataset was downloaded from the Allen Mouse Brain Atlas (portal.brain-map.org/atlases-and-data/rnaseq).

qPCR Analysis of rRNA Removal Efficiency

To assess the rRNA removal efficiency of Patho-DBiT, qPCR analysis was performed on cDNA amplicons obtained from three independent FFPE mouse E13 embryos before and after rRNA removal. Each sample, with an input amount of 2.5 ng cDNA, underwent a total volume of 25 μL in the KAPA HiFi HotStart ReadyMix reaction system. Forward and reverse primers targeting cytoplasmic (5S, 5.8S, 18S, and 28S) and mitochondrial (12S and 16S) rRNA were custom-designed and ordered from IDT. QuantiTect Primer Assays for mouse GAPDH and β-actin genes served as internal controls. The qPCR reactions were conducted on a CFX Connect Real-Time System, and fold changes were determined using the comparative CT method.

### Gene Body Coverage Calculation

For each sample, the percentile coverage was computed along the gene body from 5′ to 3′ using the “geneBody_coverage.py” module from the RSeQC package V5.0.1 with default settings.

### Spatial Alternative Splicing Analysis

For both the Patho-DBIT FFPE mouse brain and 10× Genomics Visium FFPE or fresh-frozen mouse brain samples, five types of alternative splicing events were evaluated (SE, RI, A3SS, A5SS, MXE) and their respective splice-junction-spanning read counts from the Binary Alignment Map (BAM) file of each sample. The rMATS-turbo pipeline V4.1.2 with parameters “-t single—allow-clipping—variable-read-length” and the GRCm38-mm10 mouse gene annotation were employed for this analysis. Candidate events were considered for further analysis if their inclusion and skipping isoform read counts were both >2 when aggregated from all pixels within the sample. Within each spatial pixel, a gene was deemed to have alternative splicing information if at least one splice-junction-spanning read of either inclusion or skipping isoform was detected. To identify alternative splicing events showing regional differences in the Patho-DBiT data, pseudo-bulk BAM files of each brain region were generated by merging reads from all the pixels within the same region. Pairwise regional differential alternative splicing analysis was performed by running rMATS-turbo on the generated pseudo-bulk BAM files for each pair of two regions. An alternative splicing event was considered significant if it exhibited an exon inclusion level difference of >0.05 between two regions, with a false discovery rate (FDR) of ≤0.05. Exon inclusion levels and FDRs were obtained from rMATS-turbo's splice-junction-read-based outputs (*. MATS.JC.txt). The spatial locations of reads corresponding to alternative splicing events were deciphered using their barcode sequences, resulting in distinct inclusion and skipping isoform expression matrices for each event type. Seurat V4's “NormalizeData” with a “LogNormalize”-based global-scaling normalization was applied, and the “SpatialFeaturePlot” was employed to visualize the spatial distribution of selected isoforms.

### Spatial Adenosine-to-Inosine (A-to-1) RNA Editing Analysis

A total of 107,095 reference mouse A-to-I RNA editing sites were retrieved from the REDIportal database (srv00.recas.ba.infn.it/atlas/search_mm.html) as of the download date on Sep. 20, 2023. The counts of edited and unedited reads for each editing site were calculated from the BAM file containing all spatial pixels using the “mpileup” subcommand of samtools V1.16.1, with parameters “—no-output-ins—no-output-ins—no-output-del—no-output-del—no-output-ends-B-d 0-Q 25-q 25” along with the reference editing site list and the GRCm38-mm10 mouse reference genome. Reads with bases “A” and “G” at editing sites were classified as unedited and edited, respectively. Candidate A-to-I RNA editing sites for further analysis were defined as those with a total coverage of ≥10 and an edited read count of ≥1 when aggregated from all pixels within the sample. The overall editing ratio for each editing site was computed by dividing the total number of edited reads across all pixels by the total coverage of that site. Similarly, the average editing ratio for each pixel or brain region was determined by dividing the total edited reads by the total coverage of all editing sites within that specific area. The reference spatial dataset, containing editing sites, editing ratios, and total read counts from long-read Nanopore sequencing of fresh frozen mouse brain sections, was obtained from the literature. For comparison with the Patho-DBiT dataset, only sites with ≥10 long reads were included.

Spatial microRNA Alignment and Analysis

The transcriptome output function of STAR was used to generate the microRNA transcriptome BAM file using annotations obtained from miRBase. Only primary alignment of each read mapped to microRNA was preserved, and microRNAs with detected UMI count ≥1 were included in the downstream analysis. The nucleotide length of each mapped microRNA read was calculated and the count distribution across all identified microRNAs was generated. To visualize read coverage across the reference genomic region, the BAM file of specific microRNAs was directly imported into the Integrative Genomics Viewer (IGV), focusing on the precursor microRNA region, including the mature 5p-strand and 3p-strand, for detailed visualization. The spatial microRNA-by-pixel expression matrix was generated by decoding barcode sequences, and standard functions integrated into Seurat V4 were utilized for normalization and spatial visualization.

### Spatial Single Nucleotide Variant (SNV) Analysis

The germline variant calling pipeline, Strelka V2.9.10, was utilized to identify potential SNVs from the mapped BAM file. Only high-confidence variant loci marked as “PASS” in Strelka, along with SNV sites having sequencing counts ≥60, were retained for further analysis. Each pixel and SNV site were assigned values: 0 for wild type, 1 for heterozygous mutation, or 2 for homozygous mutation. Positions with no detected mutated nucleotides were labeled as wild type, those with both mutated and wild-type nucleotides were classified as heterozygous mutation, and sites with only mutated nucleotides were categorized as homozygous mutation. For each pixel, only SNV sites identified by Strelka were incorporated into the profile, considering that RNA-seq data may not cover the entire genome. By combining spatial coordinates defined by barcode sequences, a mutation-by-pixel matrix was generated, and the cumulative number of SNVs within each pixel was calculated to delineate spatial mutational burden. Subsequently, this mutational matrix was input into the Seurat V4 pipeline to perform unsupervised clustering analysis using standard normalization, dimensional reduction, and spatial visualization methods.

Coverage Comparison with 10× Genomics Datasets

To assess and compare the genomic location coverage bandwidth between Patho-DBIT and 10× Genomics 3′ scRNA-seq or Visium spatial datasets, the aligned BAM files were obtained from the respective website. The sequencing depth was normalized by randomly selecting an equivalent number of reads in each 10× Genomics file and the Patho-DBIT data. Genomic regions with at least one detected read were considered covered.

### Spatial RNA Splicing Dynamics

The analysis involved extracting counts of spliced and unspliced reads independently from the aligned BAM file. Genomic regions corresponding to exons and introns were obtained from the GENCODE annotation. Utilizing the “intersect” tool within bedtools V2.31.0, reads overlapping with intronic regions were identified, and the associations between each read and its corresponding gene were documented. The remaining reads that overlapped with exonic regions were selected, and their connections to the overlapped genes were documented as well. After demultiplexing their spatial coordinates, reads containing region records were processed to generate spliced and unspliced count matrices, respectively. Following this, the two matrices were imported into the sc Velo pipeline, where RNA velocity, pseudotime analysis, and visualization were implemented using default settings. Pixel annotations, featuring assigned cluster identities, were transferred from the Seurat clustering analysis conducted on the combined exonic and intronic expression matrices.

### Ligand-Receptor Interaction Analysis

The R toolkit Connectome V1.0.0 was employed to investigate cell-cell connectivity patterns using ligand and receptor expressions from the Patho-DBiT datasets. The normalized Seurat object served as input, and cluster identities were utilized to define nodes in the interaction networks, resulting in an edgelist connecting pairs of nodes through specific ligand-receptor mechanisms. The top-ranked interaction pairs were selected, prioritizing those more likely to be biologically and statistically significant based on the scaled weights of each pair. The “sources.include” and “targets.include” parameters were applied to specify the source cluster emitting ligand signals and the target cluster expressing receptor genes that sense the ligands.

### Ingenuity Pathway Analysis

Ingenuity Pathway Analysis (IPA, QIAGEN) was employed to uncover the underlying signaling pathways regulated by the DEGs characterizing each identified cluster or two groups. The DEG list, along with the corresponding fold change value, p-value, and adjusted p-value of each gene, was imported into the software. The Ingenuity Knowledge Base (genes only) served as the reference set for performing Core Expression Analysis. The z-score was utilized to assess the activation or inhibition level of specific pathways. Conceptually, the z-score is a statistical measure gauging how closely the actual expression pattern of molecules in the DEG dataset aligns with the expected pattern based on the literature for a particular annotation. A z-score >0 signifies activation or upregulation, while a z-score <0 indicates inhibition or downregulation. A z-score ≥2 or ≤−2 is considered significant. The p-value for each identified signaling pathway is calculated using the right-tailed Fisher's Exact Test. This significance reflects the probability of the association of molecules from the Patho-DBiT dataset with the canonical pathway reference dataset. Additionally, a graphical summary (FIG. 31) was generated to provide an overview of the major biological themes in the IPA Core Analysis and illustrate how these concepts interrelate. A machine learning algorithm, relying entirely on prior knowledge, was deployed to score inferred relationships between molecules, functions, and pathways. Networks were constructed from the IPA analysis results using a heuristic graph algorithm.

### Statistical Analysis

Statistical analyses were performed using Prism V9 (GraphPad), with the specific tests employed indicated.

TABLE 1
DNA barcode sequences

| | Sequence | SEQ ID NO: |
|---|---|---|
| 1st Barcode | | |
| Barcode A-1 | /5Phos/AGGCCAGAGCATTCGAACGTGATAGATGTGTATAAGAGAC | 1 |
| | AG | |
| Barcode A-2 | /5Phos/AGGCCAGAGCATTCGAAACATCGAGATGTGTATAAGAGAC | 2 |
| | AG | |
| Barcode A-3 | /5Phos/AGGCCAGAGCATTCGATGCCTAAAGATGTGTATAAGAGACA | 3 |
| | G | |
| Barcode A-4 | /5Phos/AGGCCAGAGCATTCGAGTGGTCAAGATGTGTATAAGAGAC | 4 |
| | AG | |
| Barcode A-5 | /5Phos/AGGCCAGAGCATTCGACCACTGTAGATGTGTATAAGAGACA | 5 |
| | G | |
| Barcode A-6 | /5Phos/AGGCCAGAGCATTCGACATTGGCAGATGTGTATAAGAGACA | 6 |
| | G | |
| Barcode A-7 | /5Phos/AGGCCAGAGCATTCGCAGATCTGAGATGTGTATAAGAGACA | 7 |
| | G | |
| Barcode A-8 | /5Phos/AGGCCAGAGCATTCGCATCAAGTAGATGTGTATAAGAGACA | 8 |
| | G | |
| Barcode A-9 | /5Phos/AGGCCAGAGCATTCGCGCTGATCAGATGTGTATAAGAGACA | 9 |
| | G | |
| Barcode A-10 | /5Phos/AGGCCAGAGCATTCGACAAGCTAAGATGTGTATAAGAGAC | 10 |
| | AG | |
| Barcode A-11 | /5Phos/AGGCCAGAGCATTCGCTGTAGCCAGATGTGTATAAGAGACA | 11 |
| | G | |
| Barcode A-12 | /5Phos/AGGCCAGAGCATTCGAGTACAAGAGATGTGTATAAGAGAC | 12 |
| | AG | |
| Barcode A-13 | /5Phos/AGGCCAGAGCATTCGAACAACCAAGATGTGTATAAGAGAC | 13 |
| | AG | |
| Barcode A-14 | /5Phos/AGGCCAGAGCATTCGAACCGAGAAGATGTGTATAAGAGAC | 14 |
| | AG | |
| Barcode A-15 | /5Phos/AGGCCAGAGCATTCGAACGCTTAAGATGTGTATAAGAGACA | 15 |
| | G | |
| Barcode A-16 | /5Phos/AGGCCAGAGCATTCGAAGACGGAAGATGTGTATAAGAGAC | 16 |
| | AG | |
| Barcode A-17 | /5Phos/AGGCCAGAGCATTCGAAGGTACAAGATGTGTATAAGAGAC | 17 |
| | AG | |
| Barcode A-18 | /5Phos/AGGCCAGAGCATTCGACACAGAAAGATGTGTATAAGAGAC | 18 |
| | AG | |
| Barcode A-19 | /5Phos/AGGCCAGAGCATTCGACAGCAGAAGATGTGTATAAGAGAC | 19 |
| | AG | |
| Barcode A-20 | /5Phos/AGGCCAGAGCATTCGACCTCCAAAGATGTGTATAAGAGACA | 20 |
| | G | |
| Barcode A-21 | /5Phos/AGGCCAGAGCATTCGACGCTCGAAGATGTGTATAAGAGAC | 21 |
| | AG | |
| Barcode A-22 | /5Phos/AGGCCAGAGCATTCGACGTATCAAGATGTGTATAAGAGACA | 22 |
| | G | |
| Barcode A-23 | /5Phos/AGGCCAGAGCATTCGACTATGCAAGATGTGTATAAGAGACA | 23 |
| | G | |
| Barcode A-24 | /5Phos/AGGCCAGAGCATTCGAGAGTCAAAGATGTGTATAAGAGAC | 24 |
| | AG | |
| Barcode A-25 | /5Phos/AGGCCAGAGCATTCGAGATCGCAAGATGTGTATAAGAGAC | 25 |
| | AG | |
| Barcode A-26 | /5Phos/AGGCCAGAGCATTCGAGCAGGAAAGATGTGTATAAGAGAC | 26 |
| | AG | |
| Barcode A-27 | /5Phos/AGGCCAGAGCATTCGAGTCACTAAGATGTGTATAAGAGACA | 27 |
| | G | |
| Barcode A-28 | /5Phos/AGGCCAGAGCATTCGATCCTGTAAGATGTGTATAAGAGACA | 28 |
| | G | |
| Barcode A-29 | /5Phos/AGGCCAGAGCATTCGATTGAGGAAGATGTGTATAAGAGAC | 29 |
| | AG | |
| Barcode A-30 | /5Phos/AGGCCAGAGCATTCGCAACCACAAGATGTGTATAAGAGAC | 30 |
| | AG | |
| Barcode A-31 | /5Phos/AGGCCAGAGCATTCGGACTAGTAAGATGTGTATAAGAGAC | 31 |
| | AG | |
| Barcode A-32 | /5Phos/AGGCCAGAGCATTCGCAATGGAAAGATGTGTATAAGAGAC | 32 |
| | AG | |
| Barcode A-33 | /5Phos/AGGCCAGAGCATTCGCACTTCGAAGATGTGTATAAGAGACA | 33 |
| | G | |
| Barcode A-34 | /5Phos/AGGCCAGAGCATTCGCAGCGTTAAGATGTGTATAAGAGACA | 34 |
| | G | |
| Barcode A-35 | /5Phos/AGGCCAGAGCATTCGCATACCAAAGATGTGTATAAGAGAC | 35 |
| | AG | |
| Barcode A-36 | /5Phos/AGGCCAGAGCATTCGCCAGTTCAAGATGTGTATAAGAGACA | 36 |
| | G | |
| Barcode A-37 | /5Phos/AGGCCAGAGCATTCGCCGAAGTAAGATGTGTATAAGAGAC | 37 |
| | AG | |
| Barcode A-38 | /5Phos/AGGCCAGAGCATTCGCCGTGAGAAGATGTGTATAAGAGAC | 38 |
| | AG | |
| Barcode A-39 | /5Phos/AGGCCAGAGCATTCGCCTCCTGAAGATGTGTATAAGAGACA | 39 |
| | G | |
| Barcode A-40 | /5Phos/AGGCCAGAGCATTCGCGAACTTAAGATGTGTATAAGAGACA | 40 |
| | G | |
| Barcode A-41 | /5Phos/AGGCCAGAGCATTCGCGACTGGAAGATGTGTATAAGAGAC | 41 |
| | AG | |
| Barcode A-42 | /5Phos/AGGCCAGAGCATTCGCGCATACAAGATGTGTATAAGAGAC | 42 |
| | AG | |
| Barcode A-43 | /5Phos/AGGCCAGAGCATTCGCTCAATGAAGATGTGTATAAGAGACA | 43 |
| | G | |
| Barcode A-44 | /5Phos/AGGCCAGAGCATTCGCTGAGCCAAGATGTGTATAAGAGAC | 44 |
| | AG | |
| Barcode A-45 | /5Phos/AGGCCAGAGCATTCGCTGGCATAAGATGTGTATAAGAGACA | 45 |
| | G | |
| Barcode A-46 | /5Phos/AGGCCAGAGCATTCGGAATCTGAAGATGTGTATAAGAGAC | 46 |
| | AG | |
| Barcode A-47 | /5Phos/AGGCCAGAGCATTCGCAAGACTAAGATGTGTATAAGAGAC | 47 |
| | AG | |
| Barcode A-48 | /5Phos/AGGCCAGAGCATTCGGAGCTGAAAGATGTGTATAAGAGAC | 48 |
| | AG | |
| Barcode A-49 | /5Phos/AGGCCAGAGCATTCGGATAGACAAGATGTGTATAAGAGAC | 49 |
| | AG | |
| Barcode A-50 | /5Phos/AGGCCAGAGCATTCGGCCACATAAGATGTGTATAAGAGAC | 50 |
| | AG | |
| 2nd Barcode | | |
| Barcode B-1 | /5Biosg/CAAGCGTTGGCTTCTCGCATCTNNNNNNNNNNAACGTGAT | 51 |
| | ATCCACGTGCTTGAG | |
| Barcode B-2 | /5Biosg/CAAGCGTTGGCTTCTCGCATCTNNNNNNNNNNAAACATCG | 52 |
| | ATCCACGTGCTTGAG | |
| Barcode B-3 | /5Biosg/CAAGCGTTGGCTTCTCGCATCTNNNNNNNNNNATGCCTAA | 53 |
| | ATCCACGTGCTTGAG | |
| Barcode B-4 | /5Biosg/CAAGCGTTGGCTTCTCGCATCTNNNNNNNNNNAGTGGTCA | 54 |
| | ATCCACGTGCTTGAG | |
| Barcode B-5 | /5Biosg/CAAGCGTTGGCTTCTCGCATCTNNNNNNNNNNACCACTGT | 55 |
| | ATCCACGTGCTTGAG | |
| Barcode B-6 | /5Biosg/CAAGCGTTGGCTTCTCGCATCTNNNNNNNNNNACATTGGC | 56 |
| | ATCCACGTGCTTGAG | |
| Barcode B-7 | /5Biosg/CAAGCGTTGGCTTCTCGCATCTNNNNNNNNNNCAGATCTG | 57 |
| | ATCCACGTGCTTGAG | |
| Barcode B-8 | /5Biosg/CAAGCGTTGGCTTCTCGCATCTNNNNNNNNNNCATCAAGT | 58 |
| | ATCCACGTGCTTGAG | |
| Barcode B-9 | /5Biosg/CAAGCGTTGGCTTCTCGCATCTNNNNNNNNNNCGCTGATC | 59 |
| | ATCCACGTGCTTGAG | |
| Barcode B-10 | /5Biosg/CAAGCGTTGGCTTCTCGCATCTNNNNNNNNNNACAAGCTA | 60 |
| | ATCCACGTGCTTGAG | |
| Barcode B-11 | /5Biosg/CAAGCGTTGGCTTCTCGCATCTNNNNNNNNNNCTGTAGCC | 61 |
| | ATCCACGTGCTTGAG | |
| Barcode B-12 | /5Biosg/CAAGCGTTGGCTTCTCGCATCTNNNNNNNNNNAGTACAAG | 62 |
| | ATCCACGTGCTTGAG | |
| Barcode B-13 | /5Biosg/CAAGCGTTGGCTTCTCGCATCTNNNNNNNNNNAACAACCA | 63 |
| | ATCCACGTGCTTGAG | |
| Barcode B-14 | /5Biosg/CAAGCGTTGGCTTCTCGCATCTNNNNNNNNNNAACCGAGA | 64 |
| | ATCCACGTGCTTGAG | |
| Barcode B-15 | /5Biosg/CAAGCGTTGGCTTCTCGCATCTNNNNNNNNNNAACGCTTA | 65 |
| | ATCCACGTGCTTGAG | |
| Barcode B-16 | /5Biosg/CAAGCGTTGGCTTCTCGCATCTNNNNNNNNNNAAGACGGA | 66 |
| | ATCCACGTGCTTGAG | |
| Barcode B-17 | /5Biosg/CAAGCGTTGGCTTCTCGCATCTNNNNNNNNNNAAGGTACA | 67 |
| | ATCCACGTGCTTGAG | |
| Barcode B-18 | /5Biosg/CAAGCGTTGGCTTCTCGCATCTNNNNNNNNNNACACAGAA | 68 |
| | ATCCACGTGCTTGAG | |
| Barcode B-19 | /5Biosg/CAAGCGTTGGCTTCTCGCATCTNNNNNNNNNNACAGCAGA | 69 |
| | ATCCACGTGCTTGAG | |
| Barcode B-20 | /5Biosg/CAAGCGTTGGCTTCTCGCATCTNNNNNNNNNNACCTCCAA | 70 |
| | ATCCACGTGCTTGAG | |
| Barcode B-21 | /5Biosg/CAAGCGTTGGCTTCTCGCATCTNNNNNNNNNNACGCTCGA | 71 |
| | ATCCACGTGCTTGAG | |
| Barcode B-22 | /5Biosg/CAAGCGTTGGCTTCTCGCATCTNNNNNNNNNNACGTATCA | 72 |
| | ATCCACGTGCTTGAG | |
| Barcode B-23 | /5Biosg/CAAGCGTTGGCTTCTCGCATCTNNNNNNNNNNACTATGCA | 73 |
| | ATCCACGTGCTTGAG | |
| Barcode B-24 | /5Biosg/CAAGCGTTGGCTTCTCGCATCTNNNNNNNNNNAGAGTCAA | 74 |
| | ATCCACGTGCTTGAG | |
| Barcode B-25 | /5Biosg/CAAGCGTTGGCTTCTCGCATCTNNNNNNNNNNAGATCGCA | 75 |
| | ATCCACGTGCTTGAG | |
| Barcode B-26 | /5Biosg/CAAGCGTTGGCTTCTCGCATCTNNNNNNNNNNAGCAGGAA | 76 |
| | ATCCACGTGCTTGAG | |
| Barcode B-27 | /5Biosg/CAAGCGTTGGCTTCTCGCATCTNNNNNNNNNNAGTCACTA | 77 |
| | ATCCACGTGCTTGAG | |
| Barcode B-28 | /5Biosg/CAAGCGTTGGCTTCTCGCATCTNNNNNNNNNNATCCTGTA | 78 |
| | ATCCACGTGCTTGAG | |
| Barcode B-29 | /5Biosg/CAAGCGTTGGCTTCTCGCATCTNNNNNNNNNNATTGAGGA | 79 |
| | ATCCACGTGCTTGAG | |
| Barcode B-30 | /5Biosg/CAAGCGTTGGCTTCTCGCATCTNNNNNNNNNNCAACCACA | 80 |
| | ATCCACGTGCTTGAG | |
| Barcode B-31 | /5Biosg/CAAGCGTTGGCTTCTCGCATCTNNNNNNNNNNGACTAGTA | 81 |
| | ATCCACGTGCTTGAG | |
| Barcode B-32 | /5Biosg/CAAGCGTTGGCTTCTCGCATCTNNNNNNNNNNCAATGGAA | 82 |
| | ATCCACGTGCTTGAG | |
| Barcode B-33 | /5Biosg/CAAGCGTTGGCTTCTCGCATCTNNNNNNNNNNCACTTCGA | 83 |
| | ATCCACGTGCTTGAG | |
| Barcode B-34 | /5Biosg/CAAGCGTTGGCTTCTCGCATCTNNNNNNNNNNCAGCGTTA | 84 |
| | ATCCACGTGCTTGAG | |
| Barcode B-35 | /5Biosg/CAAGCGTTGGCTTCTCGCATCTNNNNNNNNNNCATACCAA | 85 |
| | ATCCACGTGCTTGAG | |
| Barcode B-36 | /5Biosg/CAAGCGTTGGCTTCTCGCATCTNNNNNNNNNNCCAGTTCA | 86 |
| | ATCCACGTGCTTGAG | |
| Barcode B-37 | /5Biosg/CAAGCGTTGGCTTCTCGCATCTNNNNNNNNNNCCGAAGTA | 87 |
| | ATCCACGTGCTTGAG | |
| Barcode B-38 | /5Biosg/CAAGCGTTGGCTTCTCGCATCTNNNNNNNNNNCCGTGAGA | 88 |
| | ATCCACGTGCTTGAG | |
| Barcode B-39 | /5Biosg/CAAGCGTTGGCTTCTCGCATCTNNNNNNNNNNCCTCCTGA | 89 |
| | ATCCACGTGCTTGAG | |
| Barcode B-40 | /5Biosg/CAAGCGTTGGCTTCTCGCATCTNNNNNNNNNNCGAACTTA | 90 |
| | ATCCACGTGCTTGAG | |
| Barcode B-41 | /5Biosg/CAAGCGTTGGCTTCTCGCATCTNNNNNNNNNNCGACTGGA | 91 |
| | ATCCACGTGCTTGAG | |
| Barcode B-42 | /5Biosg/CAAGCGTTGGCTTCTCGCATCTNNNNNNNNNNCGCATACA | 92 |
| | ATCCACGTGCTTGAG | |
| Barcode B-43 | /5Biosg/CAAGCGTTGGCTTCTCGCATCTNNNNNNNNNNCTCAATGA | 93 |
| | ATCCACGTGCTTGAG | |
| Barcode B-44 | /5Biosg/CAAGCGTTGGCTTCTCGCATCTNNNNNNNNNNCTGAGCCA | 94 |
| | ATCCACGTGCTTGAG | |
| Barcode B-45 | /5Biosg/CAAGCGTTGGCTTCTCGCATCTNNNNNNNNNNCTGGCATA | 95 |
| | ATCCACGTGCTTGAG | |
| Barcode B-46 | /5Biosg/CAAGCGTTGGCTTCTCGCATCTNNNNNNNNNNGAATCTGA | 96 |
| | ATCCACGTGCTTGAG | |
| Barcode B-47 | /5Biosg/CAAGCGTTGGCTTCTCGCATCTNNNNNNNNNNCAAGACTA | 97 |
| | ATCCACGTGCTTGAG | |
| Barcode B-48 | /5Biosg/CAAGCGTTGGCTTCTCGCATCTNNNNNNNNNNGAGCTGAA | 98 |
| | ATCCACGTGCTTGAG | |
| Barcode B-49 | /5Biosg/CAAGCGTTGGCTTCTCGCATCTNNNNNNNNNNGATAGACA | 99 |
| | ATCCACGTGCTTGAG | |
| Barcode B-50 | /5Biosg/CAAGCGTTGGCTTCTCGCATCTNNNNNNNNNNGCCACATA | 100 |
| | ATCCACGTGCTTGAG | |

All references, patents and patent applications disclosed herein are incorporated by reference with respect to the subject matter for which each is cited, which in some cases can encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

What is claimed is:

1. A method, comprising:
(a) producing spatially barcoded complementary deoxyribonucleic acids (cDNAs) from polyadenylated fragmented ribonucleic acids (RNAs) in a tissue section obtained from formalin-fixed paraffin-embedded (FFPE) tissue; and
(b) mapping the spatially barcoded cDNAs to points of origin within the tissue section.

2. The method of claim 1, wherein (a) comprises:
(i) delivering a polyadenylate polymerase to the tissue section, and optionally delivering to the tissue section a polyadenylation reagent selected from polyadenylation specificity factors, cleavage stimulation factors, polyadenylate binding proteins, and cleavage factors;
(ii) delivering reverse transcription reagents to the tissue section; and
(iii) delivering to the tissue section a first set of barcoded polynucleotides, a second set of barcoded polynucleotides, and ligation reagents to produce spatially barcoded cDNAs.

3. A method, comprising:
(a) polyadenylating fragmented RNAs in a tissue section obtained from formalin-fixed paraffin-embedded (FFPE) tissue to produce polyadenylated RNAs;
(b) producing cDNAs from the polyadenylated RNAs;
(c) spatially barcoding the cDNAs to produce spatially barcoded cDNAs; and
(d) mapping the spatially barcoded cDNAs to points of origin within the tissue section.

4. The method of any one of the preceding claims, wherein the fragmented RNAs are selected from the group consisting of mRNAs, ribosomal RNAs, transfer RNAs, microRNAs, long noncoding RNAs, small noncoding RNAs, small nuclear RNA, and piwi RNAs.

5. The method of claim 3 or 4, wherein (a) comprises delivering a polyadenylate polymerase to the tissue section, and optionally delivering to the tissue section a polyadenylation reagent selected from polyadenylation specificity factors, cleavage stimulation factors, polyadenylate binding proteins, and cleavage factors.

6. The method of any one of claims 3-5, wherein (b) comprises delivering reverse transcription reagents to the tissue section.

7. The method of any one of claims 3-6, wherein (c) comprises delivering to the tissue section a first set of barcoded polynucleotides, a second set of barcoded polynucleotides, and ligation reagents to produce the spatially barcoded cDNAs.

8. A method, comprising:
(a) delivering a polyadenylate polymerase to a tissue section obtained from formalin-fixed paraffin-embedded (FFPE) tissue to produce polyadenylated ribonucleic acids (RNAs);
(b) delivering reverse transcription reagents to the tissue section to produce cDNAs;
(c) delivering to the tissue section a first set of barcoded polynucleotides, a second set of barcoded polynucleotides, and ligation reagents to produce spatially barcoded cDNAs;
(e) imaging the tissue section to produce a sample image;
(f) sequencing the spatially barcoded cDNAs to produce sequencing reads; and
(g) mapping the spatially barcoded cDNAs to points of origin within the tissue section.

9. The method of claim 8, wherein (a) further comprises delivering to the tissue section a polyadenylation reagent selected from polyadenylation specificity factors, cleavage stimulation factors, polyadenylate binding proteins, and cleavage factors.

10. The method of claim 8 or 9, wherein the imaging is with an optical microscope or a fluorescence microscope.

11. The method of any one of claims 2, 6, and 7, wherein the first set of barcoded polynucleotides and the second set of barcoded polynucleotides are delivered using a microfluidic device, optionally made from polydimethylsiloxane (PDMS).

12. The method of claim 11, wherein the microfluidic device comprises a first component for delivery of the first set of barcoded polynucleotides and a second component for delivery of the second set of barcoded polynucleotides, each of the components comprising parallel variable width microchannels.

13. The method of any one of the preceding claims, wherein the tissue section has been permeabilized.

14. The method of claim 13, wherein the tissue section was frozen prior to being permeabilized.

15. The method of any one of the preceding claims, wherein the tissue section is mounted on a microscope slide.

16. The method of any one of the preceding claims, wherein the FFPE tissue is mammalian tissue, optionally human tissue.

17. The method of any one of the preceding claims, wherein the FFPE tissue is bacterial tissue.

18. The method of claim 17, wherein each of the first component and the second component comprises 5-50 variable width microchannels, each of the microchannels having (i) an inlet port and an outlet port, (ii) a width of 50-150 μm, at the inlet port and the outlet port, and (iii) a width of 10-50 μm at the tissue section.

19. The method of claim 17 or 18, wherein the first component and the second component are oriented at an angle of greater than 10 degrees relative to each other during delivery of the first set of barcoded polynucleotides and the second set of barcoded polynucleotides.

20. The method of claim 19, wherein the first component and the second component are oriented perpendicular relative to each other during delivery of the first set of barcoded polynucleotides and the second set of barcoded polynucleotides.

21. The method of any one of the preceding claims, wherein the mapping comprises:
(i) calculating gene expression levels based on sequencing reads;
(ii) constructing a spatial molecular expression map by correlating gene expression levels to spatial sequences within the sequencing reads; and
(iii) correlating the spatial molecular expression map to the sample image.

22. The method of claim 21, wherein calculating gene expression levels comprises aligning sequencing reads to a reference genome.

23. The method of claim 22, wherein the reference genome is derived from a mammalian genome.

24. The method of claim 23, wherein the mammalian genome is a human genome or a rodent genome.

25. The method of any one of claims 21-24, wherein constructing the spatial molecular expression map comprises generating a uniform manifold approximation and projection map (UMAP).

26. The method of any one of claims 21-25, wherein step (iii) further comprises correlating spatial sequences within the sequencing reads to locations within the sample image.

27. The method of any one of claims 2-26, wherein the first set of barcoded polynucleotides comprises a sequence having 90% sequence identity to any one of SEQ ID NOs: 1-50.

28. The method of claim 27, wherein the first set of barcoded polynucleotides comprises a sequence according to any one of SEQ ID NOs: 1-50.

29. The method of any one of claims 2-28, wherein the second set of barcoded polynucleotides comprises a sequence having 90% sequence identity to any one of SEQ ID NOs: 51-100.

30. The method of claim 29, wherein the second set of barcoded polynucleotides comprises a sequence according to any one of SEQ ID NOs: 51-100.


SEQUENCE LISTING (100 sequences)

SEQ ID NO: 1 - DNA, 42 bp, synthetic construct
  source 1..42: mol_type = other DNA
aggccagagcattcgaacgtgatagatgtgtataagagacag

SEQ ID NO: 2 - DNA, 42 bp, synthetic construct
  source 1..42: mol_type = other DNA
aggccagagcattcgaaacatcgagatgtgtataagagacag

SEQ ID NO: 3 - DNA, 42 bp, synthetic construct
  source 1..42: mol_type = other DNA
aggccagagcattcgatgcctaaagatgtgtataagagacag

SEQ ID NO: 4 - DNA, 42 bp, synthetic construct
  source 1..42: mol_type = other DNA
aggccagagcattcgagtggtcaagatgtgtataagagacag

SEQ ID NO: 5 - DNA, 42 bp, synthetic construct
  source 1..42: mol_type = other DNA
aggccagagcattcgaccactgtagatgtgtataagagacag

SEQ ID NO: 6 - DNA, 42 bp, synthetic construct
  source 1..42: mol_type = other DNA
aggccagagcattcgacattggcagatgtgtataagagacag

SEQ ID NO: 7 - DNA, 42 bp, synthetic construct
  source 1..42: mol_type = other DNA
aggccagagcattcgcagatctgagatgtgtataagagacag

SEQ ID NO: 8 - DNA, 42 bp, synthetic construct
  source 1..42: mol_type = other DNA
aggccagagcattcgcatcaagtagatgtgtataagagacag

SEQ ID NO: 9 - DNA, 42 bp, synthetic construct
  source 1..42: mol_type = other DNA
aggccagagcattcgcgctgatcagatgtgtataagagacag

SEQ ID NO: 10 - DNA, 42 bp, synthetic construct
  source 1..42: mol_type = other DNA
aggccagagcattcgacaagctaagatgtgtataagagacag

SEQ ID NO: 11 - DNA, 42 bp, synthetic construct
  source 1..42: mol_type = other DNA
aggccagagcattcgctgtagccagatgtgtataagagacag

SEQ ID NO: 12 - DNA, 42 bp, synthetic construct
  source 1..42: mol_type = other DNA
aggccagagcattcgagtacaagagatgtgtataagagacag

SEQ ID NO: 13 - DNA, 42 bp, synthetic construct
  source 1..42: mol_type = other DNA
aggccagagcattcgaacaaccaagatgtgtataagagacag

SEQ ID NO: 14 - DNA, 42 bp, synthetic construct
  source 1..42: mol_type = other DNA
aggccagagcattcgaaccgagaagatgtgtataagagacag

SEQ ID NO: 15 - DNA, 42 bp, synthetic construct
  source 1..42: mol_type = other DNA
aggccagagcattcgaacgcttaagatgtgtataagagacag

SEQ ID NO: 16 - DNA, 42 bp, synthetic construct
  source 1..42: mol_type = other DNA
aggccagagcattcgaagacggaagatgtgtataagagacag

SEQ ID NO: 17 - DNA, 42 bp, synthetic construct
  source 1..42: mol_type = other DNA
aggccagagcattcgaaggtacaagatgtgtataagagacag

SEQ ID NO: 18 - DNA, 42 bp, synthetic construct
  source 1..42: mol_type = other DNA
aggccagagcattcgacacagaaagatgtgtataagagacag

SEQ ID NO: 19 - DNA, 42 bp, synthetic construct
  source 1..42: mol_type = other DNA
aggccagagcattcgacagcagaagatgtgtataagagacag

SEQ ID NO: 20 - DNA, 42 bp, synthetic construct
  source 1..42: mol_type = other DNA
aggccagagcattcgacctccaaagatgtgtataagagacag

SEQ ID NO: 21 - DNA, 42 bp, synthetic construct
  source 1..42: mol_type = other DNA
aggccagagcattcgacgctcgaagatgtgtataagagacag

SEQ ID NO: 22 - DNA, 42 bp, synthetic construct
  source 1..42: mol_type = other DNA
aggccagagcattcgacgtatcaagatgtgtataagagacag

SEQ ID NO: 23 - DNA, 42 bp, synthetic construct
  source 1..42: mol_type = other DNA
aggccagagcattcgactatgcaagatgtgtataagagacag

SEQ ID NO: 24 - DNA, 42 bp, synthetic construct
  source 1..42: mol_type = other DNA
aggccagagcattcgagagtcaaagatgtgtataagagacag

SEQ ID NO: 25 - DNA, 42 bp, synthetic construct
  source 1..42: mol_type = other DNA
aggccagagcattcgagatcgcaagatgtgtataagagacag

SEQ ID NO: 26 - DNA, 42 bp, synthetic construct
  source 1..42: mol_type = other DNA
aggccagagcattcgagcaggaaagatgtgtataagagacag

SEQ ID NO: 27 - DNA, 42 bp, synthetic construct
  source 1..42: mol_type = other DNA
aggccagagcattcgagtcactaagatgtgtataagagacag

SEQ ID NO: 28 - DNA, 42 bp, synthetic construct
  source 1..42: mol_type = other DNA
aggccagagcattcgatcctgtaagatgtgtataagagacag

SEQ ID NO: 29 - DNA, 42 bp, synthetic construct
  source 1..42: mol_type = other DNA
aggccagagcattcgattgaggaagatgtgtataagagacag

SEQ ID NO: 30 - DNA, 42 bp, synthetic construct
  source 1..42: mol_type = other DNA
aggccagagcattcgcaaccacaagatgtgtataagagacag

SEQ ID NO: 31 - DNA, 42 bp, synthetic construct
  source 1..42: mol_type = other DNA
aggccagagcattcggactagtaagatgtgtataagagacag

SEQ ID NO: 32 - DNA, 42 bp, synthetic construct
  source 1..42: mol_type = other DNA
aggccagagcattcgcaatggaaagatgtgtataagagacag

SEQ ID NO: 33 - DNA, 42 bp, synthetic construct
  source 1..42: mol_type = other DNA
aggccagagcattcgcacttcgaagatgtgtataagagacag

SEQ ID NO: 34 - DNA, 42 bp, synthetic construct
  source 1..42: mol_type = other DNA
aggccagagcattcgcagcgttaagatgtgtataagagacag

SEQ ID NO: 35 - DNA, 42 bp, synthetic construct
  source 1..42: mol_type = other DNA
aggccagagcattcgcataccaaagatgtgtataagagacag

SEQ ID NO: 36 - DNA, 42 bp, synthetic construct
  source 1..42: mol_type = other DNA
aggccagagcattcgccagttcaagatgtgtataagagacag

SEQ ID NO: 37 - DNA, 42 bp, synthetic construct
  source 1..42: mol_type = other DNA
aggccagagcattcgccgaagtaagatgtgtataagagacag

SEQ ID NO: 38 - DNA, 42 bp, synthetic construct
  source 1..42: mol_type = other DNA
aggccagagcattcgccgtgagaagatgtgtataagagacag

SEQ ID NO: 39 - DNA, 42 bp, synthetic construct
  source 1..42: mol_type = other DNA
aggccagagcattcgcctcctgaagatgtgtataagagacag

SEQ ID NO: 40 - DNA, 42 bp, synthetic construct
  source 1..42: mol_type = other DNA
aggccagagcattcgcgaacttaagatgtgtataagagacag

SEQ ID NO: 41 - DNA, 42 bp, synthetic construct
  source 1..42: mol_type = other DNA
aggccagagcattcgcgactggaagatgtgtataagagacag

SEQ ID NO: 42 - DNA, 42 bp, synthetic construct
  source 1..42: mol_type = other DNA
aggccagagcattcgcgcatacaagatgtgtataagagacag

SEQ ID NO: 43 - DNA, 42 bp, synthetic construct
  source 1..42: mol_type = other DNA
aggccagagcattcgctcaatgaagatgtgtataagagacag

SEQ ID NO: 44 - DNA, 42 bp, synthetic construct
  source 1..42: mol_type = other DNA
aggccagagcattcgctgagccaagatgtgtataagagacag

SEQ ID NO: 45 - DNA, 42 bp, synthetic construct
  source 1..42: mol_type = other DNA
aggccagagcattcgctggcataagatgtgtataagagacag

SEQ ID NO: 46 - DNA, 42 bp, synthetic construct
  source 1..42: mol_type = other DNA
aggccagagcattcggaatctgaagatgtgtataagagacag

SEQ ID NO: 47 - DNA, 42 bp, synthetic construct
  source 1..42: mol_type = other DNA
aggccagagcattcgcaagactaagatgtgtataagagacag

SEQ ID NO: 48 - DNA, 42 bp, synthetic construct
  source 1..42: mol_type = other DNA
aggccagagcattcggagctgaaagatgtgtataagagacag

SEQ ID NO: 49 - DNA, 42 bp, synthetic construct
  source 1..42: mol_type = other DNA
aggccagagcattcggatagacaagatgtgtataagagacag

SEQ ID NO: 50 - DNA, 42 bp, synthetic construct
  source 1..42: mol_type = other DNA
aggccagagcattcggccacataagatgtgtataagagacag

SEQ ID NO: 51 - DNA, 55 bp, synthetic construct
  source 1..55: mol_type = other DNA
caagcgttggcttctcgcatctnnnnnnnnnnaacgtgatatccacgtgcttgag

SEQ ID NO: 52 - DNA, 55 bp, synthetic construct
  source 1..55: mol_type = other DNA
caagcgttggcttctcgcatctnnnnnnnnnnaaacatcgatccacgtgcttgag

SEQ ID NO: 53 - DNA, 55 bp, synthetic construct
  source 1..55: mol_type = other DNA
caagcgttggcttctcgcatctnnnnnnnnnnatgcctaaatccacgtgcttgag

SEQ ID NO: 54 - DNA, 55 bp, synthetic construct
  source 1..55: mol_type = other DNA
caagcgttggcttctcgcatctnnnnnnnnnnagtggtcaatccacgtgcttgag

SEQ ID NO: 55 - DNA, 55 bp, synthetic construct
  source 1..55: mol_type = other DNA
caagcgttggcttctcgcatctnnnnnnnnnnaccactgtatccacgtgcttgag

SEQ ID NO: 56 - DNA, 55 bp, synthetic construct
  source 1..55: mol_type = other DNA
caagcgttggcttctcgcatctnnnnnnnnnnacattggcatccacgtgcttgag

SEQ ID NO: 57 - DNA, 55 bp, synthetic construct
  source 1..55: mol_type = other DNA
caagcgttggcttctcgcatctnnnnnnnnnncagatctgatccacgtgcttgag

SEQ ID NO: 58 - DNA, 55 bp, synthetic construct
  source 1..55: mol_type = other DNA
caagcgttggcttctcgcatctnnnnnnnnnncatcaagtatccacgtgcttgag

SEQ ID NO: 59 - DNA, 55 bp, synthetic construct
  source 1..55: mol_type = other DNA
caagcgttggcttctcgcatctnnnnnnnnnncgctgatcatccacgtgcttgag

SEQ ID NO: 60 - DNA, 55 bp, synthetic construct
  source 1..55: mol_type = other DNA
caagcgttggcttctcgcatctnnnnnnnnnnacaagctaatccacgtgcttgag

SEQ ID NO: 61 - DNA, 55 bp, synthetic construct
  source 1..55: mol_type = other DNA
caagcgttggcttctcgcatctnnnnnnnnnnctgtagccatccacgtgcttgag

SEQ ID NO: 62 - DNA, 55 bp, synthetic construct
  source 1..55: mol_type = other DNA
caagcgttggcttctcgcatctnnnnnnnnnnagtacaagatccacgtgcttgag

SEQ ID NO: 63 - DNA, 55 bp, synthetic construct
  source 1..55: mol_type = other DNA
caagcgttggcttctcgcatctnnnnnnnnnnaacaaccaatccacgtgcttgag

SEQ ID NO: 64 - DNA, 55 bp, synthetic construct
  source 1..55: mol_type = other DNA
caagcgttggcttctcgcatctnnnnnnnnnnaaccgagaatccacgtgcttgag

SEQ ID NO: 65 - DNA, 55 bp, synthetic construct
  source 1..55: mol_type = other DNA
caagcgttggcttctcgcatctnnnnnnnnnnaacgcttaatccacgtgcttgag

SEQ ID NO: 66 - DNA, 55 bp, synthetic construct
  source 1..55: mol_type = other DNA
caagcgttggcttctcgcatctnnnnnnnnnnaagacggaatccacgtgcttgag

SEQ ID NO: 67 - DNA, 55 bp, synthetic construct
  source 1..55: mol_type = other DNA
caagcgttggcttctcgcatctnnnnnnnnnnaaggtacaatccacgtgcttgag

SEQ ID NO: 68 - DNA, 55 bp, synthetic construct
  source 1..55: mol_type = other DNA
caagcgttggcttctcgcatctnnnnnnnnnnacacagaaatccacgtgcttgag

SEQ ID NO: 69 - DNA, 55 bp, synthetic construct
  source 1..55: mol_type = other DNA
caagcgttggcttctcgcatctnnnnnnnnnnacagcagaatccacgtgcttgag

SEQ ID NO: 70 - DNA, 55 bp, synthetic construct
  source 1..55: mol_type = other DNA
caagcgttggcttctcgcatctnnnnnnnnnnacctccaaatccacgtgcttgag

SEQ ID NO: 71 - DNA, 55 bp, synthetic construct
  source 1..55: mol_type = other DNA
caagcgttggcttctcgcatctnnnnnnnnnnacgctcgaatccacgtgcttgag

SEQ ID NO: 72 - DNA, 55 bp, synthetic construct
  source 1..55: mol_type = other DNA
caagcgttggcttctcgcatctnnnnnnnnnnacgtatcaatccacgtgcttgag

SEQ ID NO: 73 - DNA, 55 bp, synthetic construct
  source 1..55: mol_type = other DNA
caagcgttggcttctcgcatctnnnnnnnnnnactatgcaatccacgtgcttgag

SEQ ID NO: 74 - DNA, 55 bp, synthetic construct
  source 1..55: mol_type = other DNA
caagcgttggcttctcgcatctnnnnnnnnnnagagtcaaatccacgtgcttgag

SEQ ID NO: 75 - DNA, 55 bp, synthetic construct
  source 1..55: mol_type = other DNA
caagcgttggcttctcgcatctnnnnnnnnnnagatcgcaatccacgtgcttgag

SEQ ID NO: 76 - DNA, 55 bp, synthetic construct
  source 1..55: mol_type = other DNA
caagcgttggcttctcgcatctnnnnnnnnnnagcaggaaatccacgtgcttgag

SEQ ID NO: 77 - DNA, 55 bp, synthetic construct
  source 1..55: mol_type = other DNA
caagcgttggcttctcgcatctnnnnnnnnnnagtcactaatccacgtgcttgag

SEQ ID NO: 78 - DNA, 55 bp, synthetic construct
  source 1..55: mol_type = other DNA
caagcgttggcttctcgcatctnnnnnnnnnnatcctgtaatccacgtgcttgag

SEQ ID NO: 79 - DNA, 55 bp, synthetic construct
  source 1..55: mol_type = other DNA
caagcgttggcttctcgcatctnnnnnnnnnnattgaggaatccacgtgcttgag

SEQ ID NO: 80 - DNA, 55 bp, synthetic construct
  source 1..55: mol_type = other DNA
caagcgttggcttctcgcatctnnnnnnnnnncaaccacaatccacgtgcttgag

SEQ ID NO: 81 - DNA, 55 bp, synthetic construct
  source 1..55: mol_type = other DNA
caagcgttggcttctcgcatctnnnnnnnnnngactagtaatccacgtgcttgag

SEQ ID NO: 82 - DNA, 55 bp, synthetic construct
  source 1..55: mol_type = other DNA
caagcgttggcttctcgcatctnnnnnnnnnncaatggaaatccacgtgcttgag

SEQ ID NO: 83 - DNA, 55 bp, synthetic construct
  source 1..55: mol_type = other DNA
caagcgttggcttctcgcatctnnnnnnnnnncacttcgaatccacgtgcttgag

SEQ ID NO: 84 - DNA, 55 bp, synthetic construct
  source 1..55: mol_type = other DNA
caagcgttggcttctcgcatctnnnnnnnnnncagcgttaatccacgtgcttgag

SEQ ID NO: 85 - DNA, 55 bp, synthetic construct
  source 1..55: mol_type = other DNA
caagcgttggcttctcgcatctnnnnnnnnnncataccaaatccacgtgcttgag

SEQ ID NO: 86 - DNA, 55 bp, synthetic construct
  source 1..55: mol_type = other DNA
caagcgttggcttctcgcatctnnnnnnnnnnccagttcaatccacgtgcttgag

SEQ ID NO: 87 - DNA, 55 bp, synthetic construct
  source 1..55: mol_type = other DNA
caagcgttggcttctcgcatctnnnnnnnnnnccgaagtaatccacgtgcttgag

SEQ ID NO: 88 - DNA, 55 bp, synthetic construct
  source 1..55: mol_type = other DNA
caagcgttggcttctcgcatctnnnnnnnnnnccgtgagaatccacgtgcttgag

SEQ ID NO: 89 - DNA, 55 bp, synthetic construct
  source 1..55: mol_type = other DNA
caagcgttggcttctcgcatctnnnnnnnnnncctcctgaatccacgtgcttgag

SEQ ID NO: 90 - DNA, 55 bp, synthetic construct
  source 1..55: mol_type = other DNA
caagcgttggcttctcgcatctnnnnnnnnnncgaacttaatccacgtgcttgag

SEQ ID NO: 91 - DNA, 55 bp, synthetic construct
  source 1..55: mol_type = other DNA
caagcgttggcttctcgcatctnnnnnnnnnncgactggaatccacgtgcttgag

SEQ ID NO: 92 - DNA, 55 bp, synthetic construct
  source 1..55: mol_type = other DNA
caagcgttggcttctcgcatctnnnnnnnnnncgcatacaatccacgtgcttgag

SEQ ID NO: 93 - DNA, 55 bp, synthetic construct
  source 1..55: mol_type = other DNA
caagcgttggcttctcgcatctnnnnnnnnnnctcaatgaatccacgtgcttgag

SEQ ID NO: 94 - DNA, 55 bp, synthetic construct
  source 1..55: mol_type = other DNA
caagcgttggcttctcgcatctnnnnnnnnnnctgagccaatccacgtgcttgag

SEQ ID NO: 95 - DNA, 55 bp, synthetic construct
  source 1..55: mol_type = other DNA
caagcgttggcttctcgcatctnnnnnnnnnnctggcataatccacgtgcttgag

SEQ ID NO: 96 - DNA, 55 bp, synthetic construct
  source 1..55: mol_type = other DNA
caagcgttggcttctcgcatctnnnnnnnnnngaatctgaatccacgtgcttgag

SEQ ID NO: 97 - DNA, 55 bp, synthetic construct
  source 1..55: mol_type = other DNA
caagcgttggcttctcgcatctnnnnnnnnnncaagactaatccacgtgcttgag

SEQ ID NO: 98 - DNA, 55 bp, synthetic construct
  source 1..55: mol_type = other DNA
caagcgttggcttctcgcatctnnnnnnnnnngagctgaaatccacgtgcttgag

SEQ ID NO: 99 - DNA, 55 bp, synthetic construct
  source 1..55: mol_type = other DNA
caagcgttggcttctcgcatctnnnnnnnnnngatagacaatccacgtgcttgag

SEQ ID NO: 100 - DNA, 55 bp, synthetic construct
  source 1..55: mol_type = other DNA
caagcgttggcttctcgcatctnnnnnnnnnngccacataatccacgtgcttgag